SEQUENCE LISTING (128 sequences)

SEQ ID NO: 1 - protein, 230 aa, Artificial Sequence
  Synthetic
  MISC_FEATURE (1)..(14): optionally absent
LALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPAFQQQLQKAKAEFAQHQK

SEQ ID NO: 2 - protein, 230 aa, Artificial Sequence
  Synthetic
  MISC_FEATURE (1)..(14): optionally absent
LALVATGNDTTTKPDLYYLKNSEAINSLALLPPPPAVGSIAFLNDQAMYEQGRLLRNTERGKLAAEDANLSPEGVANAFSCAFGSPITPKDAPALYKLLRNMIEDAGDLATRSAKDHYMRIRPFAFYGVSTCNTTEQDKLSKNGSYPSGHTSIGWAFALVLAEINPQRQNEILKRGYELGQSRVICGYHWQSDVDAARVVGSAVVATLHTNPEFQAQLIKAKIEFKQHQK

SEQ ID NO: 3 - protein, 242 aa, Artificial Sequence
  Synthetic
  MISC_FEATURE (1)..(24): optionally absent
  MISC_FEATURE (241)..(242): optionally absent
MGHHHHHHGGLALVATGNDTTTKPDLYYLKNSEAINSLALLPPPPAVGSIAFLNDQAMYEQGRLLRNTERGKLAAEDANLSPEGVANAFSCAFGSPITPKDAPALYKLLRNMIEDAGDLATRSAKDHYMRIRPFAFYGVSTCNTTEQDKLSKNGSYPSGHTSIGWAFALVLAEINPQRQNEILKRGYELGQSRVICGYHWQSDVDAARVVGSAVVATLHTNPEFQAQLIKAKIEFKQHQKEL

SEQ ID NO: 4 - protein, 230 aa, Artificial Sequence
  Synthetic
  MISC_FEATURE (1)..(14): optionally absent
LALVATGNDATTKPDLYYLKNSEAINSLALLPPPPAVGSIAFLNDQAMYEQGRLLRNTERGKLAAEDANLSPEHVANAFSCAFGSPITPKDAPALYKLLRNMIEDAGDLATRSAKDHYMRIRPFAFYGVSTCNTTEQDKLSKNGSYPSGHTSIGWAMALVLAEINPDRQNEILKRGYELGQSRVICGYHWQSDVDAARVVGSAVVATLHTNPEFQAQLIKAKIEFKQHQK

SEQ ID NO: 5 - protein, 242 aa, Artificial Sequence
  Synthetic
  MISC_FEATURE (1)..(24): optionally absent
  MISC_FEATURE (241)..(242): optionally absent
MGHHHHHHGGLALVATGNDATTKPDLYYLKNSEAINSLALLPPPPAVGSIAFLNDQAMYEQGRLLRNTERGKLAAEDANLSPEHVANAFSCAFGSPITPKDAPALYKLLRNMIEDAGDLATRSAKDHYMRIRPFAFYGVSTCNTTEQDKLSKNGSYPSGHTSIGWAMALVLAEINPDRQNEILKRGYELGQSRVICGYHWQSDVDAARVVGSAVVATLHTNPEFQAQLIKAKIEFKQHQKEL

SEQ ID NO: 6 - protein, 230 aa, Artificial Sequence
  Synthetic
  MISC_FEATURE (1)..(14): optionally absent
LALVATGNDATTKPDLYYLKNSEAINSLALLPPPPAVGSIAFLNDQAMYEQGRLLRNTERGKLAQEDANLSPEQVAKAFSCAFGFPITPEDAPALYKLLRNMIEDAGDLATRSAKDHYQRIRPFAFYGVSTCNTTEQDKLSKNGSYPSGHTAIGWAFALVLAEINPDRQNEILKRGYELGQSRVICGYHWQSDVDAGRVVGSAVVATLHTNPEFQAQLIKAKIEFKQHQK

SEQ ID NO: 7 - protein, 242 aa, Artificial Sequence
  Synthetic
  MISC_FEATURE (1)..(24): optionally absent
  MISC_FEATURE (241)..(242): optionally absent
MGHHHHHHGGLALVATGNDATTKPDLYYLKNSEAINSLALLPPPPAVGSIAFLNDQAMYEQGRLLRNTERGKLAQEDANLSPEQVAKAFSCAFGFPITPEDAPALYKLLRNMIEDAGDLATRSAKDHYQRIRPFAFYGVSTCNTTEQDKLSKNGSYPSGHTAIGWAFALVLAEINPDRQNEILKRGYELGQSRVICGYHWQSDVDAGRVVGSAVVATLHTNPEFQAQLIKAKIEFKQHQKEL

SEQ ID NO: 8 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 9 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPAFIAALIKALILTHRHRT

SEQ ID NO: 10 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHRNPAFIAALIKALILTYRHRT

SEQ ID NO: 11 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPKFISALIKALILTVRHRT

SEQ ID NO: 12 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHRNPKFISALIKALILTYRHRT

SEQ ID NO: 13 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPKFISALIKALILTYRHRT

SEQ ID NO: 14 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPKFISALIKALILTHRHRT

SEQ ID NO: 15 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPEFQAQLIKALILTYRHRT

SEQ ID NO: 16 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPEFQAQLIKALILTHRHRT

SEQ ID NO: 17 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHRNPEFQAQLIKALILTYRHRT

SEQ ID NO: 18 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPKFISALIEALLEFLEHRQ

SEQ ID NO: 19 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPEFQAQLIEALLEFLEHRQ

SEQ ID NO: 20 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPAFIAALIKAKILFAEHQK

SEQ ID NO: 21 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPKFISALIKAKILFAEHQK

SEQ ID NO: 22 - protein, 222 aa, Artificial Sequence; identical to SEQ ID NO: 21
  Synthetic
  MISC_FEATURE (1)..(8): optionally absent

SEQ ID NO: 23 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPDFQQALIKALLEFAEHQK

SEQ ID NO: 24 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPDFQQALIKALLEFHEHQK

SEQ ID NO: 25 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPKFRSNLIRAKIEFKRHQK

SEQ ID NO: 26 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPKFRSNLIRAKIEFTRHQK

SEQ ID NO: 27 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPKFRSNLIRAKIEFARHQK

SEQ ID NO: 28 - protein, 222 aa, Artificial Sequence; identical to SEQ ID NO: 25
  Synthetic
  MISC_FEATURE (1)..(8): optionally absent

SEQ ID NO: 29 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPEFISNLIEAKIEFVDHQK

SEQ ID NO: 30 - protein, 222 aa, Artificial Sequence
  Synthetic
  MISC_FEATURE (1)..(8): optionally absent
GNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTQDKLSKNGSYPSGHTSIGWATALVLAEINPQRQNEILKRGYELGQSRVICGYHWQSDVDAARVVGSAVVATLHTNPEFQVQLIEALEEFTEHQK

SEQ ID NO: 31 - protein, 599 aa, Artificial Sequence
  Synthetic
  MISC_FEATURE (1)..(8): optionally absent
MGHHHHHHSGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGGSGGSGGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQKFRQQPPPPQQSGGNDATTKPDLYYLKNSEAINSLALLPPPPAVGSIAFLNDQAMYEQGRLLRNTERGKLAQEDANLSPEQVAKAFSCAFGFPITPEDAPALYKLLRNMIEDAGDLATRSAKDHYQRIRPFAFYGVSTCNTTEQDKLSKNGSYPSGHTAIGWAFALVLAEINPDRQNEILKRGYELGQSRVICGYHWQSDVDAGRVVGSAVVATLHTNPEFQAQLIKAKIEFKQHQK

SEQ ID NO: 32 - protein, 480 aa, Artificial Sequence
  Synthetic
  MISC_FEATURE (1)..(8): optionally absent
MGHHHHHHGENLYFQGVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKGSGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 33 - protein, 481 aa, Artificial Sequence
  Synthetic
  MISC_FEATURE (1)..(8): optionally absent
MGHHHHHHGENLYFQGMVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHRGSGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 34 - protein, 466 aa, Artificial Sequence
  Synthetic
  ′MISC_FEATURE (1)..(8): optionally absent
MGHHHHHHGENLYFQGVSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQMKTTYKAAKEILEMPGDHYIGHRLVRKTEGNITEQVEDAVAHSGSGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 35 - protein, 486 aa, Artificial Sequence
  Synthetic
  MISC_FEATURE (1)..(8): optionally absent
MGHHHHHHGENLYFQGMVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNGSGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 36 - protein, 485 aa, Artificial Sequence
  Synthetic
  MISC_FEATURE (1)..(8): optionally absent
MGHHHHHHGENLYFQGSKGEELFTGVVPILVELDGDVNGHKFSVRGEGEGDATNGKLTLKFICTTGKLPVPWPTLVTTLSWGVQCFARYPDHMKQHDFFKSAMPEGYVQERTISFKDDGTYKTRAEVKFEGDTLVNRIELKGIDFKEDGNILGHKLEYNYFSDNVYITADKQKNGIKANFKIRHNVEDGSVQLADHYQQNTPIGDGPVLLPDNHYLSTQSVLSKDPNEKRDHMVLLEFVTAAGITHGMDELYGSGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 37 - protein, 468 aa, Artificial Sequence
  Synthetic
  MISC_FEATURE (1)..(8): optionally absent
MGHHHHHHGENLYFQGELIKENMHMKLYMEGTVNNHHFKATSEGEGKPYEGTQTMRIKVVEGGPLPFAFDILATSFMYGSRTFIKHTQGIPDFFKQSFPEGFTWERVTTYEDGGVLTATQDTSLQDGMLIYNVKIRGVNFPSNGPVMQKKTLGWEANTEMLYPADGGLEGRSDMALKLVGGGHLIVNFKTTYRSKKPAKNLKMPGVYYVDHRLERIKEADKETYVEQHEVAVARYGSGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 38 - protein, 485 aa, Artificial Sequence
  Synthetic
  MISC_FEATURE (1)..(8): optionally absent
MGHHHHHHGENLYFQGSKGEELFTGVVPILVELDGDVNGHKFSVRGEGEGDATNGKLTLKLICTTGKLPVPWPTLVTTLGYGVQCFARYPDHMKQHDFFKSAMPEGYVQERTISFKDDGTYKTRAEVKFEGDTLVNRIELKGIDFKEDGNILGHKLEYNYNSHNVYITADKQKNGIKANFKIRHNVEDGSVQLADHYQQNTPIGDGPVLLPDNHYLSYQSVLSKDPNEKRDHMVLLEFVTAAGITHGMDELYGSGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 39 - protein, 356 aa, Artificial Sequence
  Synthetic
  MISC_FEATURE (1)..(8): optionally absent
MGHHHHHHSGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGGSGGSGGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 40 - protein, 478 aa, Artificial Sequence
  Synthetic
  MISC_FEATURE (1)..(8): optionally absent
MGHHHHHHGSSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYMGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 41 - protein, 461 aa, Artificial Sequence
  Synthetic
  MISC_FEATURE (1)..(8): optionally absent
MGHHHHHHGSSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGSGKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 42 - protein, 502 aa, Artificial Sequence
  Synthetic
  MISC_FEATURE (1)..(8): optionally absent
MGHHHHHHGSENLYFQGSGGLNDIFEAQKIEWHESKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYMGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 43 - protein, 467 aa, Artificial Sequence
  Synthetic
  MISC_FEATURE (1)..(8): optionally absent
MGHHHHHHMVSAIKPDMRIKLRMEGNVNGHHFVIDGDGTGKPYEGKQTMDLEVKEGGPLPFAFDILTTAFHYGNRVFVKYPDNIQDYFKQSFPKGYSWERSLTFEDGGICYARNDITMEGDTFYNKVRFYGTNFPANGPVMQKKTLKWEPSTEKMYVRDGVLTGDIHMALLLEGNAHYRCDFRTTYKAKEKGVKLPGYHFVDHAIEILSHDKDYNKVKLYEHAVAHSGLPDNARRSGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 44 - protein, 421 aa, Artificial Sequence
  Synthetic
  MISC_FEATURE (1)..(8): optionally absent
MGHHHHHHMDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 45 - protein, 364 aa, Artificial Sequence
  Synthetic
  MISC_FEATURE (1)..(8): optionally absent
MGHHHHHHGSENLYFQGSGHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPKGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 46 - protein, 487 aa, Artificial Sequence
  Synthetic
  MISC_FEATURE (7)..(12): optionally absent
NKIHGSHHHHHHGSTSGSAMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKGGSGGSLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQK

SEQ ID NO: 47 - protein, 631 aa, Artificial Sequence
  Synthetic
  MISC_FEATURE (1)..(8): optionally absent
MGHHHHHHSGSGENLYFQGSGPSRLEELRRRLTEPGSLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQKFRQQPPPPQQSGGNDATTKPDLYYLKNSEAINSLALLPPPPAVGSIAFLNDQAMYEQGRLLRNTERGKLAQEDANLSPEQVAKAFSCAFGFPITPEDAPALYKLLRNMIEDAGDLATRSAKDHYQRIRPFAFYGVSTCNTGSGSGGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGSGSGSGSGQDKLSKNGSYPSGHTAIGWAFALVLAEINPDRQNEILKRGYELGQSRVICGYHWQSDVDAGRVVGSAVVATLHTNPEFQAQLIKAKIEFKQHQK

SEQ ID NO: 48 - protein, 230 aa, Artificial Sequence; identical to SEQ ID NO: 1
  Synthetic

SEQ ID NO: 49 - protein, 242 aa, Artificial Sequence
  Synthetic
MGHHHHHHGGLALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPEFQAQLIKAKIEFKQHQKEL

SEQ ID NO: 50 - protein, 197 aa, Artificial Sequence
  Synthetic
  MISC_FEATURE (1)..(2): optionally absent
  MISC_FEATURE (190)..(197): optionally absent
MGSLITLVELEWLEHQLIVQLSERLKGQIAKVGELLCECLKKGGKILICGNGGSAADAQHFAAELSGRYKKERKALAGIALTTDTSALSAIGNDYGFEFVFSRQVEALGNEKDVLIGISTSGKSPNVLEALKKAKELNMLCLGLSGKGGGMMNKLCDHNLVVPSDDTARIQEMHILIIHTLCQIIDESFLEHHHHHH

SEQ ID NO: 51 - protein, 187 aa, Artificial Sequence
  Synthetic
SLITLVELEWLEHQLIVQLSERLKGQIAKVGELLCECLKKGGKILICGNGGSAADAQHFAAELSGRYKKERKALAGIALTTDTSALSAIGNDYGFEFVFSRQVEALGNEKDVLIGISTSGKSPNVLEALKKAKELNMLCLGLSGKGGGMMNKLCDHNLVVPSDDTARIQEMHILIIHTLCQIIDESF

SEQ ID NO: 52 - protein, 197 aa, Artificial Sequence
  Synthetic
  MISC_FEATURE (1)..(2): optionally absent
  MISC_FEATURE (190)..(197): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKAKELNMLCLGLSGKGGGKMNKLCDHNLVVPSDDTARIQEMHILIIHTLCQIIDEAFLEHHHHHH

SEQ ID NO: 53 - protein, 187 aa, Artificial Sequence
  Synthetic
SLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKAKELNMLCLGLSGKGGGKMNKLCDHNLVVPSDDTARIQEMHILIIHTLCQIIDEAF

SEQ ID NO: 54 - protein, 197 aa, Artificial Sequence
  Synthetic
  MISC_FEATURE (1)..(2): optionally absent
  MISC_FEATURE (190)..(197): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFLEHHHHHH

SEQ ID NO: 55 - protein, 187 aa, Artificial Sequence
  Synthetic
SLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAF

SEQ ID NO: 56 - protein, 197 aa, Artificial Sequence
  Synthetic
  MISC_FEATURE (1)..(2): optionally absent
  MISC_FEATURE (190)..(197): optionally absent
MGSLITLVELEWLEHQLIVQLSERLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAFLEHHHHHH

SEQ ID NO: 57 - protein, 187 aa, Artificial Sequence
  Synthetic
SLITLVELEWLEHQLIVQLSERLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 58 - protein, 197 aa, Artificial Sequence
  Synthetic
  MISC_FEATURE (1)..(2): optionally absent
  MISC_FEATURE (190)..(197): optionally absent
MGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFELHHHHHH

SEQ ID NO: 59 - protein, 187 aa, Artificial Sequence
  Synthetic
SLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 60 - protein, 188 aa, Artificial Sequence
  Synthetic
MSLITLVELEWLEHQLIVQLSERLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 61 - protein, 188 aa, Artificial Sequence
  Synthetic
SSLIALVHLEWLEAQLATVLSSSLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 62 - protein, 188 aa, Artificial Sequence
  Synthetic
SSLIALVYLEWLEAQLATVLSSSLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 63 - protein, 188 aa, Artificial Sequence
  Synthetic
SSLIALVELEWLEAQLATVNSSDLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 64 - protein, 188 aa, Artificial Sequence
  Synthetic
SSLIALVYLEWLEAQLATVNSSDLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 65 - protein, 188 aa, Artificial Sequence; identical to SEQ ID NO: 64
  Synthetic

SEQ ID NO: 66 - protein, 188 aa, Artificial Sequence
  Synthetic
SSLIALVHLEWLEAQLATVNSSDLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 67 - protein, 188 aa, Artificial Sequence
  Synthetic
SSLIALVYLEWLEAQLATQLSSRLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 68 - protein, 188 aa, Artificial Sequence
  Synthetic
SSLIALVHLEWLEAQLATQLSSRLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 69 - protein, 188 aa, Artificial Sequence; identical to SEQ ID NO: 67
  Synthetic

SEQ ID NO: 70 - protein, 188 aa, Artificial Sequence
  Synthetic
SSLITLVEREWIRHQLAVQNSSDLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 71 - protein, 188 aa, Artificial Sequence
  Synthetic
SSLITLVEREWIRHQLAVQLSSRLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 72 - protein, 188 aa, Artificial Sequence
  Synthetic
SSLIALVELEWLRHQLAVVLSSSLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 73 - protein, 188 aa, Artificial Sequence
  Synthetic
SSLIALVELEWLRHQLAVVDSSDLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 74 - protein, 188 aa, Artificial Sequence
  Synthetic
SSLIALVELEWLRHQLAVVNSSDLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 75 - protein, 188 aa, Artificial Sequence
  Synthetic
ASLIQLVELEWIKHQLIVQASARLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 76 - protein, 188 aa, Artificial Sequence
  Synthetic
ASLIQLVHLEWIKHQLIVQASARLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 77 - protein, 188 aa, Artificial Sequence
  Synthetic
SSLITLVELEWLAHQLAVELSSSLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 78 - protein, 188 aa, Artificial Sequence
  Synthetic
SSLITLVEEEWLEHQLAVELSSSLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 79 - protein, 188 aa, Artificial Sequence
  Synthetic
SSLITLVEEEWLAHQLAVEDSSSLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 80 - protein, 188 aa, Artificial Sequence
  Synthetic
SSLITLVEEEWLAHQLAVENSSSLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 81 - protein, 188 aa, Artificial Sequence
  Synthetic
KSLIQLVEKEWLKHQLAVVRSERLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 82 - protein, 188 aa, Artificial Sequence
  Synthetic
ASLIQLVELEWIKHQLAVQRSARLKGQIAKVGELLCRALKNGGKILICGNGGSAADAQHFAAELSGRYKKERKALAGIALTTDTSALSAIGNDYGFEFVFSRQVEALGNEGDVLIGISTSGKSPNVLEALKKARELGMLCIGLSGKGGGKMNDLCDHCLVVPSDDTARIQEMHILIIHTLCQIIDEAF

SEQ ID NO: 83 - protein, 437 aa, Artificial Sequence
  Synthetic
  MISC_FEATURE (1)..(1): optionally absent
  MISC_FEATURE (432)..(437): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGSGSSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYLEHHHHHH

SEQ ID NO: 84 - protein, 216 aa, Artificial Sequence
  Synthetic
  MISC_FEATURE (1)..(1): optionally absent
  MISC_FEATURE (211)..(216): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGSGSGAHIVMVDAYKPTKGLEHHHHHH

SEQ ID NO: 85 - protein, 441 aa, Artificial Sequence
  Synthetic
  MISC_FEATURE (1)..(1): optionally absent
  MISC_FEATURE (436)..(441): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGSGSGSGGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKLEHHHHHH

SEQ ID NO: 86 - protein, 437 aa, Artificial Sequence
  Synthetic
  MISC_FEATURE (1)..(1): optionally absent
  MISC_FEATURE (432)..(437): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGSGSGSGGSVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKLEHHHHHH

SEQ ID NO: 87 - protein, 438 aa, Artificial Sequence
  Synthetic
  MISC_FEATURE (1)..(1): optionally absent
  MISC_FEATURE (433)..(438): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGSGSGSGGSMVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHRLEHHHHHH

SEQ ID NO: 88 - protein, 423 aa, Artificial Sequence
  Synthetic
  MISC_FEATURE (1)..(1): optionally absent
  MISC_FEATURE (418)..(423): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGSGSGSGGSVSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQMKTTYKAAKEILEMPGDHYIGHRLVRKTEGNITEQVEDAVAHSLEHHHHHH

SEQ ID NO: 89 - protein, 443 aa, Artificial Sequence
  Synthetic
  MISC_FEATURE (1)..(1): optionally absent
  MISC_FEATURE (438)..(443): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGSGSGSGGSMVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNLEHHHHHH

SEQ ID NO: 90 - protein, 442 aa, Artificial Sequence
  Synthetic
  MISC_FEATURE (1)..(1): optionally absent
  MISC_FEATURE (437)..(442): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGSGSGSGGSSKGEELFTGVVPILVELDGDVNGHKFSVRGEGEGDATNGKLTLKFICTTGKLPVPWPTLVTTLSWGVQCFARYPDHMKQHDFFKSAMPEGYVQERTISFKDDGTYKTRAEVKFEGDTLVNRIELKGIDFKEDGNILGHKLEYNYFSDNVYITADKQKNGIKANFKIRHNVEDGSVQLADHYQQNTPIGDGPVLLPDNHYLSTQSVLSKDPNEKRDHMVLLEFVTAAGITHGMDELYLEHHHHHH

SEQ ID NO: 91 - protein, 425 aa, Artificial Sequence
  Synthetic
  MISC_FEATURE (1)..(1): optionally absent
  MISC_FEATURE (420)..(425): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGSGSGSGGSELIKENMHMKLYMEGTVNNHHFKATSEGEGKPYEGTQTMRIKVVEGGPLPFAFDILATSFMYGSRTFIKHTQGIPDFFKQSFPEGFTWERVTTYEDGGVLTATQDTSLQDGMLIYNVKIRGVNFPSNGPVMQKKTLGWEANTEMLYPADGGLEGRSDMALKLVGGGHLIVNFKTTYRSKKPAKNLKMPGVYYVDHRLERIKEADKETYVEQHEVAVARYLEHHHHHH

SEQ ID NO: 92 - protein, 633 aa, Artificial Sequence
  Synthetic
  MISC_FEATURE (1)..(1): optionally absent
  MISC_FEATURE (628)..(633): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGGKDRNGGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGSGSGGSVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKLEHHHHHH

SEQ ID NO: 93 - protein, 634 aa, Artificial Sequence
  Synthetic
  MISC_FEATURE (1)..(1): optionally absent
  MISC_FEATURE (629)..(634): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGGKDRNGGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGSGSGGSMVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHRLEHHHHHH

SEQ ID NO: 94 - protein, 619 aa, Artificial Sequence
  Synthetic
  MISC_FEATURE (1)..(1): optionally absent
  MISC_FEATURE (614)..(619): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGGKDRNGGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGSGSGGSVSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQMKTTYKAAKEILEMPGDHYIGHRLVRKTEGNITEQVEDAVAHSLEHHHHHH

SEQ ID NO: 95 - protein, 639 aa, Artificial Sequence
  Synthetic
  MISC_FEATURE (1)..(1): optionally absent
  MISC_FEATURE (634)..(639): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGGKDRNGGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGSGSGGSMVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNLEHHHHHH

SEQ ID NO: 96 - protein, 638 aa, Artificial Sequence
  Synthetic
  MISC_FEATURE (1)..(1): optionally absent
  MISC_FEATURE (633)..(638): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGGKDRNGGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGSGSGGSSKGEELFTGVVPILVELDGDVNGHKFSVRGEGEGDATNGKLTLKFICTTGKLPVPWPTLVTTLSWGVQCFARYPDHMKQHDFFKSAMPEGYVQERTISFKDDGTYKTRAEVKFEGDTLVNRIELKGIDFKEDGNILGHKLEYNYFSDNVYITADKQKNGIKANFKIRHNVEDGSVQLADHYQQNTPIGDGPVLLPDNHYLSTQSVLSKDPNEKRDHMVLLEFVTAAGITHGMDELYLEHHHHHH

SEQ ID NO: 97 - protein, 621 aa, Artificial Sequence
  Synthetic
  MISC_FEATURE (1)..(1): optionally absent
  MISC_FEATURE (616)..(621): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGGKDRNGGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGSGSGGSELIKENMHMKLYMEGTVNNHHFKATSEGEGKPYEGTQTMRIKVVEGGPLPFAFDILATSFMYGSRTFIKHTQGIPDFFKQSFPEGFTWERVTTYEDGGVLTATQDTSLQDGMLIYNVKIRGVNFPSNGPVMQKKTLGWEANTEMLYPADGGLEGRSDMALKLVGGGHLIVNFKTTYRSKKPAKNLKMPGVYYVDHRLERIKEADKETYVEQHEVAVARYLEHHHHHH

SEQ ID NO: 98 - protein, 638 aa, Artificial Sequence
  Synthetic
  MISC_FEATURE (1)..(1): optionally absent
  MISC_FEATURE (633)..(638): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGGKDRNGGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGSGSGGSSKGEELFTGVVPILVELDGDVNGHKFSVRGEGEGDATNGKLTLKLICTTGKLPVPWPTLVTTLGYGVQCFARYPDHMKQHDFFKSAMPEGYVQERTISFKDDGTYKTRAEVKFEGDTLVNRIELKGIDFKEDGNILGHKLEYNYNSHNVYITADKQKNGIKANFKIRHNVEDGSVQLADHYQQNTPIGDGPVLLPDNHYLSYQSVLSKDPNEKRDHMVLLEFVTAAGITHGMDELYLEHHHHHH

SEQ ID NO: 99 - protein, 636 aa, Artificial Sequence
  Synthetic
  MISC_FEATURE (1)..(1): optionally absent
  MISC_FEATURE (631)..(636): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGGKDRNGGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGGSGTSGSAMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKLEHHHHHH

SEQ ID NO: 100 - protein, 188 aa, Artificial Sequence
  Synthetic
MSLINLVEKEWQEHQKIVQASEILKGQIAKVGELLCECLKKGGKILICGNGGSAADAQHFAAELSGRYKKERKALAGIALTTDTSALSAIGNDYGFEFVFSRQVEALGNEKDVLIGISTSGKSPNVLEALKKAKELNMLCLGLSGKGGGMMNKLCDHNLVVPSDDTARIQEMHILIIHTLCQIIDESF

SEQ ID NO: 101 - protein, 392 aa, Artificial Sequence
  Synthetic
  MISC_FEATURE (1)..(1): optionally absent
  MISC_FEATURE (387)..(392): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGSGGSGGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFLEHHHHHH

SEQ ID NO: 102 - protein, 640 aa, Artificial Sequence
  Synthetic
  MISC_FEATURE (1)..(1): optionally absent
  MISC_FEATURE (635)..(640): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGETSSKQDLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGSGSGSGMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITLGMDELYKLEHHHHHH

SEQ ID NO: 103 - protein, 633 aa, Artificial Sequence; identical to SEQ ID NO: 92
  Synthetic
  MISC_FEATURE (1)..(1): optionally absent
  MISC_FEATURE (628)..(633): optionally absent

SEQ ID NO: 104 - protein, 517 aa, Artificial Sequence
  Synthetic
  MISC_FEATURE (1)..(1): optionally absent
  MISC_FEATURE (512)..(517): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGETSSKQDLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGSGSGSGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGLEHHHHHH

SEQ ID NO: 105 - protein, 635 aa, Artificial Sequence
  Synthetic
  MISC_FEATURE (1)..(1): optionally absent
  MISC_FEATURE (630)..(635): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGGKDRNGGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGSGSGGSSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGLYLEHHHHHH

SEQ ID NO: 106 - protein, 637 aa, Artificial Sequence
  Synthetic
  MISC_FEATURE (1)..(1): optionally absent
  MISC_FEATURE (632)..(637): optionally absent
MGSLITLVELEWLEHQLIVQLSERLKGQIAKVGELLCECLKNGGKILICGNGGSAADAQHFAAELSGRYKKERKALAGIALTTDTSALSAIGNDYGFEFVFSRQVEALGNEGDVLIGISTSGKSPNVLEALKKARELNMLCIGLSGKGGGKMNDLCDHNLVVPSDDTARIQEMHILIIHTLCQIIDEAFGGGKDRNGGSLITLVELEWLEHQLIVQLSERLKGQIAKVGELLCRALKNGGKILICGNGGSAADAQHFAAELSGRYKKERKALAGIALTTDTSALSAIGNDYGFEYVFARQVEALGNEGDVLIGISTSGKSPNVLEALKKARELGMLCIGLSGKGGGKMNDLCDHCLVVPSDDTARIQEMHILIIHTLCQIIDEAFGSGSGSGGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKLEHHHHHH

SEQ ID NO: 107 - protein, 197 aa, Artificial Sequence; identical to SEQ ID NO: 50
  Synthetic

SEQ ID NO: 108 - protein, 197 aa, Artificial Sequence; identical to SEQ ID NO: 52
  Synthetic

SEQ ID NO: 109 - protein, 197 aa, Artificial Sequence; identical to SEQ ID NO: 54
  Synthetic

SEQ ID NO: 110 - protein, 197 aa, Artificial Sequence; identical to SEQ ID NO: 56
  Synthetic

SEQ ID NO: 111 - protein, 197 aa, Artificial Sequence; identical to SEQ ID NO: 58
  Synthetic

SEQ ID NO: 112 - protein, 17 aa, Artificial Sequence
  Synthetic
IDEAFGGGSGGSSLITL

SEQ ID NO: 113 - protein, 18 aa, Artificial Sequence
  Synthetic
IDEAFGGGKDRNGGSLIT

SEQ ID NO: 114 - protein, 17 aa, Artificial Sequence
  Synthetic
IDEAFTGDAGETSLITL

SEQ ID NO: 115 - protein, 19 aa, Artificial Sequence
  Synthetic
IDEAFGGETSSKQDLITLV

SEQ ID NO: 116 - protein, 46 aa, Artificial Sequence
  Synthetic
KQHQKFRQQPPPPQQSGGLALVATGNDATTKPDLYYLKNSEAINSL

SEQ ID NO: 117 - protein, 46 aa, Artificial Sequence
  Synthetic
KQHQKDKTPEDSTRSEYKGGLALVATGNDATTKPDLYYLKNSEAIN

SEQ ID NO: 118 - protein, 46 aa, Artificial Sequence
  Synthetic
KQHQKSEPQEVSETQEVPGNDATTKPDLYYLKNSEAINSLALLPPP

SEQ ID NO: 119 - protein, 46 aa, Artificial Sequence
  Synthetic
KQHQKESTKSWPPTSPAYYLKNSEAINSLALLPPPPAVGSIAFLND

SEQ ID NO: 120 - protein, 46 aa, Artificial Sequence
  Synthetic
KQHQKQQQEERQTDKKPDLYYLKNSEAINSLALLPPPPAVGSIAFL

SEQ ID NO: 121 - protein, 46 aa, Artificial Sequence
  Synthetic
KQHQKDSESSGEPGAYYLKNSEAINSLALLPPPPAVGSIAFLNDQA

SEQ ID NO: 122 - protein, 46 aa, Artificial Sequence
  Synthetic
KQHQKSRDDDKGAKHKPKKPDLYYLKNSEAINSLALLPPPPAVGSI

SEQ ID NO: 123 - protein, 46 aa, Artificial Sequence
  Synthetic
KQHQKSDSKEEEKKKSSDNSSTPGNDATTKPDLYYLKNSEAINSLA

SEQ ID NO: 124 - protein, 46 aa, Artificial Sequence
  Synthetic
KQHQKKPDERSSSKKEEDKKDRGGLALVATGNDATTKPDLYYLKNS

SEQ ID NO: 125 - protein, 46 aa, Artificial Sequence
  Synthetic
KQHQKGSGSGSGSGSGKPDLYYLKNSEAINSLALLPPPPAVGSIAF

SEQ ID NO: 126 - protein, 46 aa, Artificial Sequence
  Synthetic
KQHQKGSGSGSGSGSGSGGNDATTKPDLYYLKNSEAINSLALLPPP

SEQ ID NO: 127 - protein, 46 aa, Artificial Sequence
  Synthetic
KQHQKGSGSGSGSGSGSGSGLALVATGNDATTKPDLYYLKNSEAIN

SEQ ID NO: 128 - DNA, 21 bp, Artificial Sequence
  Synthetic
taaagaaggagatatcatatg

(19) United States
(12) Patent Application Publication
(10) Pub. No.: US 2023/0287402 A1
(43) Pub. Date: Sep. 14, 2023

(54) TWO COMPONENT CO-ASSEMBLING TWO DIMENSIONAL PROTEIN STRUCTURES

(71) Applicants: University of Washington, Seattle, WA (US); MEDICAL RESEARCH COUNCIL, AS PART OF UNITED KINGDOM RESEARCH AND INNOVATION, Swindon (GB)

(72) Inventors: Ariel Ben SASSON, Seattle, WA (US); David BAKER, Seattle, WA (US); William SHEFFLER, Seattle, WA (US); Hannele RUOHOLA-BAKER, Seattle, WA (US); Logeshwaran SOMASUNDARM, Seattle, WA (US); Emmanuel DERIVERY, Swindon (GB); Joseph WATSON, Swindon (GB)

(21) Appl. No.: 18/040,684

(22) PCT Filed: Aug. 23, 2021
(86) PCT No.: PCT/US2021/047136

§ 371 Date: Feb. 6, 2023

Related U.S. Application Data
(60) Provisional application No. 63/069,901, filed on Aug. 25, 2020.

(51) Int. Cl. C12N 15/11; C12N 15/63

(52) U.S. Cl. CPC C12N 15/11; C12N 15/63

(57) ABSTRACT

The disclosure provides two-dimensional protein structures including first and second polypeptides that are different, each form homo-oligomers, and interact to form a rigid interface, polypeptide components of such two-dimensional protein structures, and uses thereof.

### CROSS REFERENCE

This application claims priority to U.S. Provisional Patent Application Ser. No. 63/069,901 filed Aug. 25, 2020, incorporated by reference herein in its entirety.

### FEDERAL FUNDING STATEMENT

This invention was made with government support under Grant Nos. P01 GM081619 and R01 GM083867 and R01 GM097372 and U01 HL099993 and U01 HL099997, awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Aug. 22, 2021 having the file name “20-776-WO-SeqList_ST25.txt” and is 318 kb in size.

### BACKGROUND

Genetically programmable materials that spontaneously assemble into ordered structures following mixture of two or more components are far more controllable than materials constitutively forming from one component; they offer temporal control over the assembly process, thereby enabling rigorous characterization and opening up a wide variety of applications. Most previously known ordered protein 2D materials primarily involve single protein components. A de-novo interface design between rigid domains that is stabilized by extensive noncovalent interactions would provide more control over atomic structure and a robust starting point for further structural and functional modulation.

### SUMMARY

In a first aspect, the disclosure provides two-dimensional protein structures, comprising a first polypeptide and a second polypeptide, wherein
(a) the first polypeptide and the second polypeptide are different;
(b) the first polypeptide self-assembles into a first homo-oligomer, wherein the first homo-oligomer comprises a first interface region, said first interface region having a rotational symmetry;
(c) the second polypeptide self-assembles into a second homo-oligomer, wherein the second homo-oligomer comprises a second interface region, said second interface region having a rotational symmetry; and
(d) the first homo-oligomer and the second homo-oligomer interact via the first interface region and the second interface region to form a rigid interface.

In one embodiment, the disclosure provides two-dimensional protein structures, comprising a first polypeptide and a second polypeptide, wherein
(a) the first polypeptide and the second polypeptide are different;
(b) the first polypeptide self-assembles into a first homo-oligomer;
(c) the second polypeptide self-assembles into a second homo-oligomer;
(d) the first homo-oligomer and the second homo-oligomer interact to form a rigid interface; and wherein
(e) one or both of the first homo-oligomer and the second homo-oligomer has a cyclic pseudo-dihedral symmetry.

In one embodiment, the first interface region and the second interface regions comprise alpha-helical domains. In another embodiment, the interface comprises an interface between an alpha-helical domain of the first polypeptide and an alpha-helical domain of the second polypeptide. In a further embodiment, each of the first polypeptide and the second polypeptide comprise a plurality (2, 3, 4, 5, 6, 7, or more) alpha helical domains separate by loop domains. In one embodiment, the interface comprises (a) a region of the first polypeptide within 25 amino acids from the first polypeptide C-terminus, and (b) a region of the second polypeptide within 25 amino acids from the second polypeptide N-terminus. In one embodiment, the first polypeptide comprises a secondary structure as shown below, wherein positions in parentheses are optional and may be present or absent:

First polypeptide
(LLLLLLLLLLLLLL)LLLLLLHHHLLLHHHHLLLLLLLLLHHHHHHHHH
HHHHHHHLLLHHHHHHHHHHHLLHHHHHHHHHHHHLLLLLLLLLHHHHHH
HHHLHHHHHHHHHHHHHHHHLLLLHHHHHLLLLLLLLLLLLLLLLLLLLL
HHHHHHHHHHHHHHHHLHHHHHHHHHHHHHHHHHHHHHLLLLHHHHHHHH
HHHHHHHHHHHHLHHHHHHHHHHHHHHHHHLL;

and
(b) the second polypeptide comprises a secondary structure as shown below

Second polypeptide
LLHHHHHHHHHHHHHHHHHHHHHHHHHHHHHHHHHHHHHHLLLLEEEEEL
HHHHHHHHHHHHHHHLLLLLLLLLLEEELLLLHHHHHHHHHHLLHHHLLH
HHHHHHLLLLLEEEEEELLLLLHHHHHHHHHHHHLLLEEEEEEELLLHHH
HHHLLEEEEELLLLHHHHHHHHHHHHHHHHHHHHHHL

wherein H represents amino acid residues present in an alpha helix; L represents amino acids present in a loop, and E represents amino acid residues present in a beta sheet, and wherein amino acid insertions may be present in loop regions.

In other embodiment, the first polypeptide and the second polypeptides comprise polypeptides of other aspects of the disclosure.

In a second aspect, the disclosure provides polypeptides comprising an amino acid sequence having at least 50%, 55%, 60%, 65%, 70%, 75%, 80%, 85%, 90%, 91%, 92%, 93%, 94%, 95%, 96%, 97%, 98%, or 99% sequence identity to the amino acid sequence of SEQ ID NO:1, wherein the polypeptide includes a mutation at 1, 2, 3, 4, 5, 6, 7, 8, 9, 10, 11, 12, 13, 14 or all 15 positions selected from the group consisting of T210, A213, Q215, Q216, Q217. Q219, K220, K222, A223. E224, F225, A226, Q227, Q229, and K230 relative to SEQ ID NO:1, wherein residues in parentheses are optional and may be present or absent. In another embodiment, the polypeptides comprise an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity to the amino acid sequence selected from the group consisting of SEQ ID NOS:2-31, wherein residues in parentheses may be present or absent. In a further embodiment, the polypeptides may comprise one or more additional functional peptide domains. In another embodiment, the disclosure provides homo-oligomers of the polypeptide of this aspect, including but not limited to cyclic homo-oligomer.

In a third aspect, the disclosure provides polypeptides comprising an amino acid sequence having at least 50%, 55%, 60%, 65%, 70%, 75%, 80%, 85%, 90%, 91%, 92%, 93%, 94%, 95%, 96%, 97%, 98%, or 99% sequence identity to the amino acid sequence of SEQ ID NO:100, wherein the polypeptide includes a mutation at 1, 2, 3, 4, 5, 6, 7, 8, 9, 10, 11, 12, 13, or all 14 positions selected from the group consisting of M1, N5, E8, K9, Q12, E13, H14, K16, I17, V18, Q19, A20, E22, and I23 relative to SEQ ID NO:100. In one embodiment, the polypeptides comprises an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity to the amino acid sequence selected from the group consisting of SEQ ID NOS: 50-82, wherein residues in parentheses are optional and may be present or absent. In another embodiment, the polypeptides may further comprise one or more additional functional peptide domains. In another embodiment, the disclosure provides homo-oligomers of the polypeptide of this aspect, including but not limited to cyclic homo-oligomer.

In other aspects, the disclosure provides nucleic acids encoding the polypeptides of any embodiment or combination of embodiments of the disclosure, expression vectors comprising the nucleic acid of the disclosure operatively linked to a suitable promoter or other control sequence, and host cells comprising the polypeptide, nucleic acid, expression vector, and/or 2D protein material of any embodiment or combination of embodiments of the disclosure.

In one aspect, the disclosure provides 2D protein materials comprising:
(a) a first homo-oligomer comprising the homo-oligomer of any embodiment or combination of embodiments of the homo-oligomers of the second aspect of the disclosure; and
(b) a second homo-oligomer comprising the homo-oligomer of any embodiment or combination of embodiments of the homo-oligomers of the third aspect of the disclosure;
wherein the first and second homo-oligomer interact at a rigid interface. In one embodiment, the first and second homo-oligomers comprise a pair of homo-oligomers comprising an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity to the amino acid sequence selected from the group consisting of the following, wherein optional residues (including any N-terminal methionine residues) may be present or absent:
(a) SEQ ID NOS:2-7 (As1-As3), and SEQ ID NOS:50-59 (B-B4);
(b) Di_13_0A (SEQ ID NO:8) and Di_13_0B (SEQ ID NO:60);
(c) Di_13_1A (SEQ ID NO:9) and Di_13_1B (SEQ ID NO:61),
(d) Di_13_2A (SEQ ID NO:10) and Di_13_2B (SEQ ID NO:62);
(e) Di_13_3A (SEQ ID NO:11) and Di_13_3B (SEQ ID NO:63);
(f) Di_13_4A (SEQ ID NO:12) and Di_13_4B (SEQ ID NO:64);
(g) Di_13_5A (SEQ ID NO:13) and Di_13_5B (SEQ ID NO:65);
(h) Di_13_6A (SEQ ID NO:14) and Di_13_6B (SEQ ID NO:66);
(i) Di_13_7A (SEQ ID NO:15) and Di_13_7B 9 SEQ ID NO:67);
(j) Di_13_8A (SEQ ID NO:16) and Di_13_8B (SEQ ID NO:68);
(k) Di_13_9A (SEQ ID NO:17) and Di_13_9B (SEQ ID NO:69);
(I) Di_13_10A (SEQ ID NO:18) and Di_13_10B (SEQ ID NO:70);
(m) Di_13_11A (SEQ ID NO:19) and Di_13_11B (SEQ ID NO:71);
(n) Di_13_12A (SEQ ID NO:20) and Di_13_12B (SEQ ID NO:72);
(o) Di_13_13A (SEQ ID NO:21) and Di_13_13B (SEQ ID NO:73);
(p) Di_13_14A (SEQ ID NO:22) and Di_13_14B (SEQ ID NO:74);
(q) Di_13_15A (SEQ ID NO:23) and Di_13_15B (SEQ ID NO:75);
(r) Di_13_16A (SEQ ID NO:24) and Di_13_16B (SEQ ID NO:76);
(s) Di_13_17A (SEQ ID NO:25) and Di_13_17B (SEQ ID NO:77);
(t) Di_13_18A (SEQ ID NO:26) and Di_13_18B (SEQ ID NO:78);
(u) Di_13_19A (SEQ ID NO:27) and Di_13_19B (SEQ ID NO:79);
(v) Di_13_20A (SEQ ID NO:28) and Di_13_20B (SEQ ID NO:80);
(w) Di_13_21A (SEQ 1D NO:29) and Di_13_21B (SEQ ID NO:81);
(x) Di_13_22A (SEQ ID NO:30) and Di_13_22B (SEQ ID NO:82); and
(y) Cyclic A comp. (SEQ ID NO:31) and Cyclic B comp. (SEQ ID NO:101).

In another aspect, the discourse provides uses of or methods for using the polypeptides, fusion proteins, homo-polymers, 2D protein materials, nucleic acids, recombinant expression vectors, and host cells of any of the preceding claims for any suitable purpose, including but not limited to those described herein.

In a further aspect, the disclosure provides computational methods to generate de-novo binary 2D non-covalent co-assemblies by designing rigid asymmetric interfaces between two distinct protein dihedral building-blocks, optionally as further defined herein.

### DESCRIPTION OF THE FIGURES

Figure J (a-e). Design strategy and characterization of in vivo assembly. (a) Design strategy. Left: The two possible orientations of a D3 building block and 3 of the 6 possible orientations of a D2 building block compatible with p6m symmetry; symmetry axes are shown. Middle: symmetry operator arrangement in p6m; the lattice spacing degree of freedom is indicated by the dashed line (d); the corresponding building block axis is shown as dashed line on the left. Right: Example of one of the 12 possible p6m array configurations resulting from placement of the dihedral building blocks in p6m lattice, with lattice spacing parameter d indicated. (b) Generating a hethro-interface through sequence design at the contact between the two homooligomers. Left panel view direction is in-plane along the sliding axes and the right panel is rotated 90 perpendicularly to the plane. (c) Model of genetically fused GFP fused to A (AGFP). (d) Negative stain TEM images of 2D arrays formed in E. coli coexpressing A+B (top left panel) and AGFP+B (bottom left panel). The corresponding averaged images are shown in the right panel superimposed with the design model (GFP omitted). (e) Confocal microscopy images of cells coexpressing AGFP+B (left panel) or expressing only AGFP (right panel), the difference in GFP signal homogeneity suggests the arrays form within the cells, scales bar: (d) 100 nm, (e) 5 μm.

FIG. 2(a-e). Structure of in vitro assembled arrays. (a) Negative stain TEM of a single AGFP+B array. (b) Computational modeloverlayed on averaging of (a), GFP density is evident near the A N-term. (c) Negative stain TEM of micron scale arrays overnight assembly from a mixture of 5 μM components concentration in TBS supplemented with 500 mM imidazole. Insets: 1) FFT of a selected region (black rectangle), 2-3) TEM of only the A and B components, respectively. (d) Vertical projections of single (left panel) and stacked (right panel) arrays and the corresponding inferred diagram of the lattice packing arrangements. Right most panel illustrates a preferred single layer over layer order (see FIG. 13 for further details). e) SAXS profile of overnight mixtures of A and B in solution (black-circle markers) and calculated profiles from atomic models of various sheet dimensions (see FIG. 13 new (a-c) for controls. TEM validation, ASU definition and atomic models) demonstrating near identical peaks spacing and positions, indicating a very good agreement between the modelled architecture and that realized in solution. Inset to (e) shows SAXS profiles for the measured sample, 2D and 3D models (see models in FIG. 14c), implying that scattering profiles of arrays in solution belong to single layer, non stacked, 2D structures. Scale bars: (a) 200 nm; (b) 20 nm; (c) 500 nm; inset to (c) 20 nm

FIG. 3(a-d). In vitro assembly kinetics. (a) Lattice formation in solution monitored by light scattering. (b,c) AFM in-situ characterization of growth dynamics. (b) Growth (white arrow) and line defect healing (box)spanning a number of unit cells. Inset: Height section profile along the white dashed line. (c) Close up of the area showing healing of lattice vacancy defects and growth (dashed to solid white circles). (d) Lattice edge state statistics. Time indicates Scale bars: (b) 200 nm; (c) 100 nm. Elapsed time indicated in minutes.

FIG. 4(a-b). Dynamics of array-induced receptor clustering and biological activation. (a) Experimental scheme: Genetic and post-translational fusions of monomeric proteins (X) to A and B array components are used to couple multiple functions on a single structure to specifically target cells displaying a protein of choice, Xout. (b) NIH/3T3 cells expressing a fusion between an external anti-GFP nanobody (Xout=GBP), a transmembrane domain (TM) and an internal (Xin=mScarlet) domain (GBP-TM-mScarlet) were incubated with 10 μl/mL of preformed AGFP+B arrays. Note that in this experiment we used ultracentrifugation to separate arrays from free components, this results in a stack ofarrays which retain their planar order (inset to left upper panel and further details in FIG. 17). Spinning disk confocal microscopy was used to monitor mScarlet clustering followed by array contact events (2 independent events) with cells as described in (a). (c) mScarlet clustering quantification. (d) 3D rendering of mScarlet clustering underneath an array binding event over a cell membrane. (e-b) Tie2 receptor clustering induced by a binding event of preformed AfD+AGFP+B arrays (fD is the F domain of angiopoietin, not to confuse with (b-d) here the GFP functions to label the arrays), (e) arrays and Tie2 high-resolution imaging immediately after (left panel) and 15 minutes after (right panel) binding of arrays to cells. Top right inset to each panel shows the area surrounded by a dashed box omitting the array signal. Negative stain image of the arrays prior to mixing with the cells is shown in the left panel lower right inset. (f) 3D reconstruction of a no-binding event (control, left panel) and 60 minutes post binding event (right panel) showing the alignment of the array and the clustered Tie2 layer and the remodeling of the actin skeleton below the array. Split channel images and EM Control of order are shown in FIGS. 18a,b and d, respectively. (g) Tie2 clusters induced p-AKT activation. The AfD alone (col. 2-4 from the left) elicits much less AKT phosphorylation alone than when assembled into arrays by the B subunits (3 right most col.). The concentration of fD monomers in the system is 17.8 nM (×1), 53.4 nm (×3) or 89 nM (×5), as indicated. (h) Dynamics of Tie2 activation. Scale bars: (b) 3 μm; (e) 2.5 μm.

FIG. 5(a-p). Large arrays assembled on cells block endocytosis. (a) Experimental scheme: stable NIH3T3 cells constitutively expressing GBP-TM-mScarlet were incubated with 1 μM B(c)GFP, rinsed in PBS, then 0.2 μM unlabelled A was added and cells were imaged by spinning disk confocal microscopy. (b) Upon addition of A, numerous foci positive for extracellular BGFP and intracellular mScarlet appear, which eventually fuse (arrows). (c-d) Automated quantification of the effects seen in (b), (e) Size distribution (Full Width Half Maximum, FWHM) of the GFP- and mScarlet-positive spots generated in (b) imaged by TIRF microscopy (n=8972 arrays in N=50 cells). (f-g) The clustering ability of arrays is homogenous within one cell and between cells. Estimated average number of GFP and mScarlet molecules per array plotted for each cell (f mean±SEM) or for all cells as a histogram (g; n=8972 arrays in N=50 cells) imaged by TIRF microscopy. dash lines: theoretical boundary GFP/mScarlet ratios for either a 1:1 BGFP:GBP-TM-mScarlet ratio, in case both GFPs of the BGFP dimer are bound to GBP, or a 2:1 ratio, in case only one GFP of the BGFP dimer is bound to GBP. (h-i) Tuning of array size by controlling receptor density at the cell surface. Stable NIH/3T3 cells expressing GBP-TM-mScarlet under Doxycycline (Dox)-inducible promoter were treated with increased doses of Dox induction for 24 h and cells were processed as in (a). The average number of B(c)GFP molecules per array was then estimated (mean±SEM, b), as well as the GFP/mScarlet intensity ratio (i). Number of spots/cells analyzed, respectively: 0.1 μg/mL Dox: 4602/41; 0.5 μg/mL Dox: 2670/32; 2 μg/mL Dox: 6439155. Dox induction increases the number of B(c)GFP, so array size, at the cell surface, and clustering activity scales accordingly. (j) Histogram of mScarlet/GFP fluorescence intensity ratio between preformed B(c)GFP/A(d)mScarlet arrays or arrays assembled on cells by incubating stable NIH/3T3 cells constitutively expressing GBP-TM as in (a) with B(c)GFP and A(d)mScarlet (n=1058 arrays in N=12 cells/n=440 preformed arrays). The similarity of the histograms suggests that arrays assembled on cells have similar degree of order as arrays formed in vitro. (k) AFM imaging of arrays assembled similarly as on cells in (a), but on supported bilayers (see also FIG. 22). Lookup table corresponds to amplitude between 0 and 410 μm. (l) EGF receptors (EGFR) on HeLa cells were clustered (or not) using B(c)GFP, an anti-GFP-nanobody::anti-EGFR Darpin fusion (GBP-EGFR-Darpin, see methods) and A as in (a). Cells were then fixed and processed for immunofluorescence using LAMP1 antibodies and imaged by spinning disk confocal microscopy. After 40 min chase, unclustered EGFR extensively colocalizes with lysosomal marker LAMP1, while clustered EGFR stays at the plasma membrane, suggesting that array-induced 2D clustering of EGFR inhibits its endocytosis. Images correspond to maximum-intensity z-projections across entire cells (insets correspond to single confocal planes). (m) Automated object-based quantification of the effect seen (l) for the whole kinetic (n indicates number of cells analysed. Statistics were performed using an ANOVA1 test followed by a Tukey post-hoc test (p<0.001). (n-o) The endocytic block can be alleviated by tuning down the size of the arrays. (n) Stable NIH/3T3 cells expressing GBP-TM-mScarlet under Doxycyclin (Dox)-inducible promoter were treated with increasing does of Dox for 24 h, then incubated with 0.5 μM B(c)GFP, rinsed in PBS, then 0.5 μM unlabelled A was added (or not). After 60 min, cells were briefly incubated with Alexa-633-coupled Wheat Germ Agglutinin to label cell membranes, then cells were fixed and imaged by spinning disk confocal microscopy. Images correspond to single confocal planes. (o) Automated 3D quantification of the effects seen in (n), see methods. Increasing the initial array size induces a statistically significant decrease in the internalization of GFP-positive arrays. Statistics were performed using an ANOVA1 test followed by a Tukey post-hoc test (p<0.001). (p) Graphical summary illustrating the extent of the endocytic block (o) as a function of the initial mean number of B(c)GFP per array (see b). For reference, the apparent diameter of arrays as a function of their B(c)GFP content, the size of 60mer nanocages (13) and Clathrin Coated Pits (CCP) are also figured. Scale bar: 10 μm (b-left panel, l, n), 1 μm (b-right panel and I inset) and 50 nm (k).

FIG. 6(a-c). Advantages in use of dihedral symmetric building blocks for planar assemblies. (a) Model of two dihedral homooligomers, a D3 hexamer (left in gray, pair of monomers constituting a single interface) and a D2 tetramer (right in gray, with a pair of jointly interfacing monomers). Both components are positioned such that one rotation symmetry axis is aligned perpendicular to the plane (arrows) and an additional 2-fold rotation symmetry axis is aligned with one another and with the plane reflection symmetry axis (dashed line). (b) Top, front, and diagonal view of the D2 homooligomer showing the symmetric nature of the interface. Due to the C2 rotation symmetry of the interface (within each building block) it can be considered as two smaller interfaces, this is illustrated by the two diagrams showing the rotated origin. (c) We take into account that the orientation between the interfaces can vary in six ways, these are the six degrees of freedom between each two free objects in a 3D space, and could be classified to 3 translational and 3 angular DOFs. In (c) the six panels decompose the six DOFs to show the outcome of errors in each on the overall interface geometry. It shows that due to the C2 symmetry all angular deviations (lower row) and cell spacing (this is the distance between the components and illustrated here with arrows, upper left panel) are being compensated and deviations in those would not propagate along the symmetric assembly. The remaining two translation DOFs, orthogonal to the cell spacing (two rightmost upper panels) would result in an in-plane twist (red curved arrow) that if too large may hinder correct propagation.

FIG. 7. DNA translation and mRNA optimization protocol diagram. DNAworks¹, Nupack™², and mRNA optimizer³ are wrapped in a python program to optimize for protein expression in E. Coli and compatible with some typical requirements (such as GC ratio, repeat, restriction site, etc.) of providers cloning production lines. Once a desired protein sequence is obtained it is parsed to fragments of up to 200 residues (limit of DNAworks) which are passed separately to DNAworks for translation. The DNA sequences are then stitched back into a single fragment and the first n nucleotides of each gene, typically 50, are then optimized by the mRNA optimizer and Nupack™ iteratively to minimize the mRNA secondary structure ddG. The rationale is minimizing the occurrence of mRNA secondary structures which reduce the yield of protein expression by slowing or blocking the initiation and flow of the mRNA fragment through the ribosomes.³

FIG. 8. mRNA optimization to expression yield. FPLC traces (Superose™ 6 Increase 10/300 GL) of the B component. Both cultures were similarly lysed as described in methods, soluble fractions were separated using centrifugation and further purified using Ni-columns, eluted fractions were concentrated to ˜1 ml and immediately injected to the FPLC on 1 ml loops. We note that both constructs have an identical residues sequence and differ only in the DNA sequence of the first 50 nucleotides. SDS-PAGE gel in the inset to the left panel shows the bands of the corresponding fractions at the expected weight. We see an 8 fold increase in expression levels for the mRNA optimized sequences vs. the non-mRNA optimized construct.

FIG. 9. SDS-PAGE gels expression screening. For the expression analysis we use 4 columns for each design: left to right: insoluble 37° C., Insoluble 22° C., soluble 37° C., soluble 22° C. The tables aligned with each gel provide the data for each design: design number ID, location of the 6×His tag position (N/C on either component A or B), the PDB 4 digit ID of each component native model, and the expected components weight. Note that in the two columns of soluble fractions an additional band belongs to the lysozyme used for lysis.

FIG. 10(a-d). Stabilized component B constructs circular dichroism analysis. Far-ultraviolet Circular Dichroism (CD) measurements were carried out with an AVIV spectrometer, model 420. Wavelength scans were measured from 260 to 195 nm at temperatures of 25 and 95° C. Temperature melts monitored absorption signal at 222 nm in steps of 2° C./min and 30 s of equilibration time. For wavelength scans and temperature melts a protein solution in PBS buffer (pH 7.4) of concentration 0.2-0.4 mg/ml was used in a 1 mm path-length cuvette. CD spectra, wavelength (260-195 nm) scans at 25° C. (a), 95° C. (b), and 25° C. after cooling (c) are plotted as raw data (millidegrees) for B2 at 0.35 [mg/ml], B3 at 0.30 [mg/ml], and B(e) (the cyclic version of B discussed later in FIG. 18) at 0.29 [mg/ml]. (d) CD Temperature scan for 25° C. to 95° C. measured at 222 nm. Curves correspond to two stabilized versions of the dihedral B component and the B(c) component. Results show that not only the component became stable in ambient conditions but could sustain higher temperature before unfolding initiates.

FIG. 11(a-d). Stabilized component A constructs circular dichroism analysis. Panels a-d are as described in FIG. 10. Unlike the case of the B component, component A was already stable in ambient conditions and expressing well but we were interested to check if the process would allow us to obtain stability at higher temperatures that would potentially have advantages for annealing processes or storage in non-optimal conditions. As in FIG. 10, As1 to As3 are the redesigned constructs with an increasing number of mutations. In this case the protocol did not improve protein stability or thermostability except the case of construct As3. All versions behave approximately similar and exhibit high solubility at room temp.

FIG. 12(a-d). Design component solubility vs. Nearest Neighbor (NN) model. (a) Unit cell description. In the p6m plane symmetry unit cell there are exactly 2 C₃ rotation centers (triangles) and 3 C2 rotation centers (I fully within the unit cell and 4 halves, small rectangles); for illustration purposes the design model is overlaid on top of the unit cell diagram. Unit cell length is X=31 nm, and the distance between each two nearest A components or B components is denoted by d^{A}_{array} and d^{B}_{array}, respectively, and are equal to ˜15 nm and 17.5 nm, respectively. (b) Mean Nearest Neighbor distance in nm as a function of component concentrations. Based on the law of distribution of the nearest neighbor in a random distribution of particles we derive the average inter particle distance for a given component concentration, d^{A}_{NN} and d^{B}_{NN}.⁵ The mean distance is given by where, N is the number of monomers, V is volume in nm³, and Nd is the number of monomers in each homooligomer: 6 and 4 for D₃ and D₂, respectively. The vertical lines show the components distance upon assembly (d^{A}_{array} and d^{B}_{array}). Typically in our work co-assembly is initiated at components concentration around 5 μM and below (range indicated by the ellipse). The graph shows that under these concentrations the co-assembly process brings the components much closer to each other, as indicated by the two horizontal arrows. (c) NN mean distance of components stored at high concentration {D3:[2.6 μM, d^{A}_{NN}=8.7 nm], D2:[2.2 μM, d^{B}_{NN}=8.0 nm]}) is shown with a full circle markers to the left of the vertical lines thus in these concentrations d^{A}_{NN}<d^{A}_{array} and d^{B}_{NN}<d^{B}_{array}. This situation is interesting because here co-assembly practically draws the components apart, somewhat analogous to the ice/water expansion anomaly, and is substantially different from the typical process that occurs in one-component materials that assemble around a nucleation center (we note that the components are drawn apart only within the plane, unlike the situation in ice). This unique phenomenon stems from designable system properties: interface orthogonality, components stabilization, and sparse assembly geometry. (d) Illustrates of stock solution volumes required to generate a total of 1 m² of arrays. We note that in current processes multiple μM scale arrays or smaller are formed.

FIG. 13(a-e). Arrays ordered stacking. In multiple EM images either single or stack of arrays are observed. Averaging the apparently indistinguishable conformations (FIG. 11a) revealed that in all cases arrays interact through a single contact point, at the vertical faces of the B component. (b) Interacting B components from different arrays share the vertical rotation axis and are rotated around that axis by 60°, top and bottom panels show the alignment geometry from top and side views, respectively. (c) Assuming this observation defines the way the system predominantly performs means that hexagon belonging to vertically interacting arrays can interact in three different ways, all including that similar B-B interaction at exactly two contact points, rendering those three interaction options to be energetically equivalent. Thus we assume that when arrays interact all three possible options have the same probability. When an array is added to a single array all three contacting options will result in a similar outcome (panel a2 and c2). When a third and fourth layers are added, three different outcomes could be obtained (panels a and c 2-4). (d) The probabilities to observe a certain pattern given the number of arrays in a stack and support the assumption that given a hexagonal lattice is observed only a single layer is layered. (e) Given a pattern observation, the probability to have a number of stacked arrays in the observation. Again observing a hexagonal array means that only a single array is layered, while observing a square lattice does not mean that only 2 layers are stacked, even though that is the situation with the highest probability. This also shows that an observation of pattern (4) does not provide any information about the number of stacked layers. The equations above each panel describe the different probability distributions.

FIG. 14(a-g). SAXS analysis. a) Left and middle panels: Components A and B SAXS measurements (black curves) analyzed using the Scatter program and SAXS profiles for components A and B model (shown in insets), respectively) calculated using FOXS 6 and demonstrating excellent agreement (A: λ2=0.18, B: λ2=0.20) and no concentration dependence. Right panel: A+B mixture SAXS measurement (black curves) and ASU scattering profile (brown). Bragg peaks shown in the A+B SAXS data correlate with the p6 symmetry model and spacing of 303 Angstrom (see Table 3) in close agreement with TEM data and design model. The ASU model (top right panel corner) comprises 12 monomers, 6 belonging to a single A component and 6 more belonging to 3 halves of the B component. b) Negative stain TEM assembly validation for the components used for the SAXS experiments demonstrating the local expected order. c) Array models with increasing size, increasing number of ASUs, and 3D crystal model of stacked arrays as inferred from TEM analysis shown in FIG. S8. d. Scattering profiles of array models consisting of an increasing number of ASUs ([6, 9, 12, 15, 30, 36, 72, 108, 180] gray scale intensity corresponds to ASUs #) and selected models are shown in (c). A+B mixture SAXS measurement profile (as shown in (a) right panel) is shown as a black curve and circle markers demonstrating close agreement between the computational design model of the p6 array and structures formed in solution. e) Interpolation of measured arrays ASUs number and dimensions (assuming circular arrays) based on the fit to the models' SAXS profiles intensity difference between the first peak minimum and maximum (see method) suggesting that in solution (unsupported) the two components form 2D arrays which constitute about 6,000 ASUs (tera-Da scale flat assembly) and are 1.8 μm in diameter. f) SAXS profiles collected directly following the mixture of array components at time points ranging from 30 sec to 15 min. Each measurement was collected from a separate well to avoid accumulated damage to the samples. It is notable that within the first 30 seconds following components mixture at 10 μM, distinctive Bragg peaks emerge. Based on the computational model analysis (panels (e) and (g)) these newly formed arrays constitute only a few hexagons; however, this suggests that SAXS measurements enable a thorough kinetics study and construction of phase diagrams of macroscale 2D binary systems. Scale bars: 500 nm.

FIG. 15(a-d). AFM edge analysis for A+B and AGFP+B arrays. AFM arrays characterization in fluid cell on freshly cleaved mica substrate from solution containing components at equimolar concentrations of 7 uM. Arrays growth from A+B components (a left panel and b) or AGFP+B (a right panel). (c-d) edge analysis. (c-d) Edge analysis is based on our ability to characterize edge states. We show that here by comparing arrays formed from A+B components (left panels) vs. arrays formed from AGFP+B components (right panel). By analyzing the profile along crystal lattice directions (indicated with white lines in (c) and as the curves in (d)) showing a measurable signal for the GFP fusions or the lack of those. Lattice edge state analysis for the co-assembly of AGFP units and B units assuming the images capture equilibrium distributions of edge sites and are based on ΔG(i-j)=−kTln(pᵢ/pⱼ). The calculated free energy differences between different edge states: ΔG(A_{GFP}-II-A_{GFP}-I)=−5.5 kJ/mol, ΔG(B-1-A_{GFP}-I)=−5.2 kJ/mol, and ΔG(A_{GFP}-II-B)=−0.3 kJ/mol. Scales bar: (a) 200 nm, (b-c) 100 nm

FIG. 16(a-d). Preformed arrays duster transmembrane proteins in stable assemblies. (a-b) Clustering of transmembranc proteins by preformed arrays. (a) principle of the experiment: NIH/3T3 cells expressing GBP-TM-mScarlet are incubated with AGFP+B arrays for 30 min leading to clustering of the mScarlet construct. This is the same scheme as in FIG. 5a reproduced here for clarity. (b) After incubation with preformed arrays, live cells are processed for imaging by spinning disk confocal microscopy. 3D z-stacks are acquired (11 μm, Δz=0.2 μm) and processed for 3D reconstruction. Note that the intracellular mScarlet protein signal overlaps perfectly with the extracellular GFP signal of the array. (c-d) mScarlet constructs clustered by the arrays are not dynamic. (c) Cells were incubated with AGFP+B arrays for 1 hour at 37° C., then the mScarlet signal was bleached and its fluorescence recovery monitored. The GFP signal was used to delineate the bleaching area. (d) Quantification of the effect seen in a (see methods). The mScarlet signal does not recover, suggesting that GBP-TM-mScarlet molecules are stably trapped by the AGFP+B array. As a control that binding of AGFP alone (that is, not in an array) does not affect fluorescence recovery of GBP-TM-mScarlet (meaning that the array does not recover because all the GBP-TM-mScarlet is trapped by the AGFP+B array), we also performed FRAP experiments of GBP-TM-mScarlet in cells incubated with AGFP alone. As expected, this recovers. Scale bars: (b) 12 μm; (c) 6 μm.

FIG. 17(a-b). Preformed arrays clusters characterization. Negative stain TEM images of 2D arrays formed by in-vitro mixing AGFP+B in equimolar concentration (both at 5 uM) in buffer (25 mM Tris-HC, 150 mM NaCl, 5% glycerol) supplemented with 500 mM imidazole, overnight incubation at room temperature (total volume of 200 uL) in Eppendorf tube, followed by centrifugation (panel a). (b) We then remove the supernatant and resuspend the pelleted fraction in a similar buffer. Negative stain grids prepared by using a 10 fold diluted suspension buffer as described in methods and imaged in magnifications varying between ×2800 and ×28 k.

FIG. 18(a-d). Tie2 receptors clustering and CD31/VE-Cad recruitment. Imaging of cells incubated for 60 min with GFP-positive arrays functionalized with the F domain of the angiogenesis promoting factor Ang1 (a,c), or not (b), then fixed and processed for immunofluorescence with Tie2 antibodies (a,b). CD31 (c, left two panels) or VE-CAD (c, right two panels) antibodies. Note that Tie2 signal is dramatically reorganized and colocalizes with the array (compare a and b). Recruitment of CD31 and VE-Cad under the array (c, arrows), together with the extensive actin remodeling (FIG. 4f and inset to a left panel), suggests that the structure induced by the array is a precursor to adherens junction. Scale bars: (a,b,c) 2.5 μm: (d) Negative stain TEM validation of array formation using pre-functionalized components ASCSTfD+BcGFP (A component with a genetically fused spyCatcher peptide fused to spyTag-fDomain, and cyclic B component with genetically fused GFP). Scale bars: (a,b,c) 2.5 μm, (d) 500 nm.

FIG. 19(a-b). Designed cyclic pseudo-dihedral building blocks. In FIG. 6 we described the inherent pros of a dihedral symmetric building blocks for the construction of 2D, planar, assemblies owing to their pair of in-plain rotational symmetry axes. In different scenarios, however, the same pros are found to be a disadvantage. For example, attempting a stepwise assembly over soft substrates such as cell membranes (see the following FIGS. 20-24) where one of the components is initially used as an anchor, results in a failure. We presumed the reason lay in the ligand spatial distribution around the dihedral building blocks, facing both up and down (relative to the plane geometry, see example in FIG. 20a, where a GFP is used to bind to the GBP nanobodies displayed on cell membranes) such that when components bind to the cell membrane, either their orientation is such that the array assembly interfaces are blocked (FIG. 20a middle panel arrows), or the entire component becomes buried within or wrapped by the membrane (FIG. 20a right panel), thereby blocking array assembly. A useful geometry for an anchor unit in such a configuration would be one with vertical inhomogeneous binding sites, a feature inherent to cyclic components (see FIG. 20b for illustration of cyclic components binding site to a reference lipid substrate). In order to benefit from both geometries and diversify component functionality we chose to redesign the dihedral building blocks to be cyclic pseudo-dihedral ones. For example, every pair of chains in the B components which was originally a D 2 tetramer (a) are combined to a single chain, resulting in a C 2 dimer “almost”, pseudo, identical to the original homooligomer. The C 2 pD 2 array interfaces remain unchanged while ligand distribution becomes vertically inhomogeneous, therefore components could now function equally well as both anchor units and planar array building blocks (see FIGS. 5.b and 21e-f for array formation on cell membranes and controls). The computational workflow to alter the building blocks' symmetry from dihedral to cyclic pseudo-dihedral(Dx→Cx) includes a number of steps. We first use pyrosetta 11 to generate the dihedral homooligomer model and choose a pair of monomers such that their C- and N-terminus are adjacent (a simple case is shown for the B components in (a) where the C- and N-terminus are adjacent, this is not always the case as shown for component A in (b)). We then generate a set of blueprints of linkers between a set of positions near the C-terminus of one monomer and positions near the N-terminus of the second components, i.e., we truncate either or both components and suggest linker lengths and secondary structure preferences. We employ Rosetta™ Remodel 8 to generate fragments that would create ideal linkers (see illustration in (a) and (b) for different cut sites and fragments generated). We chose to test a number of linkers with either predicted rigid secondary structure or a flexible one. To generate the full constructs we cloned the linkers between two different monomers, we chose the best two stable versions of the A and B components which were generated at the stabilization process (FIGS. 10 and 11). We then expressed the proteins, now referred to as A(c) and B(c), verified monomeric weight using SDS-page, homooligomeric weight using SEC-MALS and structural functionality, ability to form similar hexagonal 2D arrays using negative stain TEM (see FIG. 20d for B(c)+A and FIG. 21d for A(c)+B and A(c)+B(c)). The Final step included genetic fusions of functional groups (GFP or SpyCatcher™ that could then be peptide fused to spy-tagged ligands) to the cyclic components N/C-terms or exposed loops. This allows a versatile set of materials to co-assemble in a stepwise fashion, i.e., first cell priming and then array assembly directly on the cell membrane, allowing controllable (timely, spatially, uniformly, and receptor or signal specific) and combinatorial experiments on cell membranes (see FIG. 5 and FIGS. 22-24).

FIG. 20(a-d). B component desymmetrization: Rationale, model, and characterization. (a) model of the B component dihedral homooligomer with GFP fusions, arrow pointing towards a perpendicular direction to the plane. Left panel illustrates that when such a dihedral homooligomer is binding to a flat surface like a lipid bilayer through GFP/GBP interactions, array interfaces are either blocked or facing a direction which is not parallel to the plane. This thereby may induce membrane wrapping and assembly block because propagation interfaces are facing the membrane. (b) model of a cyclic B component with only two GFP fusions both facing to one vertical direction. Right panel shows an ideal binding conformation with the arrows indicating the propagation direction. This does not induce any membrane remodeling. (c) schematics of the linker insertion protocol. In the D₂ dimer, C- and N-terminal ends are adjacent (left panel arrows). A linker is designed to connect the two (middle panel) resulting in a twice as big monomer which forms a C2 homooligomer. (d) negative stain EM image of array made of B(c) and A components. Scales Bars: (d) 100 nm.

FIG. 21(a-f). A component desymmetrization. (a) A component dihedral (D₃) model, two monomers and arrow pointing on the designed array interface direction. (b) Various fragments build between the C-term of one monomer to different positions near the N-term of the second monomer. (c) Model of the cyclic A component with the new linker, note that arrays interfaces were not modified. (d) negative stain TEM screening for hexagonal assemblies. Left panel shows cyclic A components with dihedral B components, while in the right panel both components are cyclic. (e-f). Cyclisation of the A-component enables array assembly on cells. Stable NIH/3T3 cells constitutively expressing GBP-TM-mScarlet were incubated with 1 μM A(d)GFP (e) or Is M A(c)GFP (f), rinsed in PBS, then 1 μM unlabelled B was added and cells were imaged by spinning disk confocal microscopy. Images correspond to a single confocal plane of the GFP channel. On the contrary to dihedral A (e), cyclic A enables rapid array assembly on cells, as seen by the characteristic appearance of diffraction limited, GFP-positive spots (see inserts and also FIG. 5 and main text). Scales Bars: (d) 100 nm, (e,f) 10 μm, 2 m for insets.

FIG. 22(a-d). Correlative SIM/AFM of arrays assembled onto supported bilayers. (a) Design of the assay (see also methods): a supported lipid bilayer containing 5% biotinylated lipids and 0.2% fluorescent lipids is formed onto a glass coverslip in a flow cell. B(c)mSA2 (20 nM) is then injected into the chamber to bind to biotinylated lipids. After washing the excess of unbound B, A(d)GFP (20 nM) is injected into the chamber. After assembly for 5 min, the chamber is extensively washed and the sample fixed. The top lid of the chamber is then removed, and the sample is imaged by Super-resolution structured illumination microscopy (SIM) imaging from the bottom and atomic force microscopy (AFM) from the top. This correlative imaging allows one to find the arrays by light microscopy, before increasing the magnification to determine their degree of order by AFM. Note that the sequential mode of assembly used here is conceptually identical to the assembly of arrays onto cells (FIG. 5). Indeed, the cyclic B component (cyclic B) is used to anchor the array to the membrane via its monovalent functionalization moiety (mSA2 here compared to GFP on cells), and assembly can only happen on the membrane, as there is no free B(c)mSA2 in solution. Accordingly, arrays assembled onto supported bilayers by this method are very similar to arrays assembled on cells when imaging with diffraction-limited microscopy (see b, left panel). (b) Low magnification image of arrays assembled as above obtained by correlative Widefield microscopy (left panel), SIM super resolution microscopy (middle panel) and AFM (right panel). Super-resolution imaging indicates that arrays appearing as diffraction-limited spots by widefield microscopy can actually be somewhat elongated structures. This is in remarkable agreement with our observation that arrays assembled on cell membranes can fuse post-assembly (FIG. 5.b and FIG. 5.d for quantification). This further confirms that assembly on supported bilayers and on cells are similar. (c) Examples of topography in the image presented in the b—right panel. Note that height measured by AFM is uniform at about 3-4 nm, confirming 2D growth. d) High-magnification images of arrays seen in (c) by fast AFM, demonstrating high hexagonal order of the polymer onto supported bilayers (see methods; Note that the bottom right panel is identical to FIG. 5.k, reproduced here for convenience). Lookup table corresponds to amplitude between 0 and 455,475 and 410 μm for the top, bottom left and bottom right panels, respectively. From b-d, we conclude that the height and the size of the lattice on membranes is exactly as expected from the design model (FIG. 1), the EM imaging of arrays assembled in solution (FIG. 2.a-c and FIG. 20, 21), the SAXS measurements of arrays assembled in solution (FIG. 2.e and FIG. 14) and the AFM measurements on mica substrates (FIG. 3 and FIG. 15). This confirms that assembly on membranes leads to ordered arrays and also validates that our quantitative light microscopy measurements (FIG. 23 and FIG. 5.j) are a valid proxy for bulk order evaluation. Scale bars: 5 μm (b) 50 nm (d).

FIG. 23(a-k). Array diffusion in cell membranes, microscope calibration curves and controls of inducible cell lines. (a) Arrays assembled onto cells slowly diffuse at the cell surface. NIH/3T3 cells expressing GBP-TM-mScarlet were treated as in FIG. 5a-b and imaged by spinning disk confocal microscopy. BGFP foci at the cell surface were then automatically tracked, and the Weighted mean Square Displacement (MSD) was plotted as a function of delay time (solid line; n:=2195 tracks in N=3 cells, lighter area: SEM). Dashed black line: linear fit reflecting diffusion (R²=0.9999; =0.0005 μm²/s). (b-e) Establishment of a 1:1 GFP/mScarlet calibration standard. (b) Purified GFP-60mer nanocages were mixed with an excess of purified GBP-mScarlet, then submitted size exclusion chromatography to isolate GFP-60mer nanocages saturated with GBP-mScarlet. (c) Chromatogram comparing the size exclusion profile of either the GFP-60mer alone, or the GFP-60mer+GBP-mScarlet mix. The high molecular weight peak of assembled 60-mer nanocages is further shifted to high molecular weight due to the extra GBP-mscarlet molecules, but is still not overlapping with the void of the column. (d) Spinning disk confocal imaging of GFP/GBP-mScarlet nanocages purified as in (e) onto a glass coverslip. Fluorescence is homogenous and there is perfect colocalization between the GFP and mscarlet channels Scale bar: 1 μm. Mean+/−SEM fluorescence in both GFP and mScarlet channels of GFP/GBP-mScarlet nanocages as a function of microscope exposure time, showing that the instrument operates in its linear range (number of particles analysed: 25 ms: n=167; 50 ms n=616; 100 ms: n=707 and 200 ms: n=1086). Similar results were obtained for TIRF microscopy. Exposure for all calibrated experiments in this paper is 50 ms. Note that the variant of GFP used throughout the paper, on both B and the nanocages is sfGFP (referred to as GFP for simplicity). (f-g) The clustering ability of arrays scales with array size and does not depend on the microscopy technique used. To explore a wide range of expression levels of GBP-TM-mScarlet, we measured the average number of GFP and mScarlet molecules per array in NIH/3T3 cells expressing GBP-TM-mScarlet either stably or transiently, leading occasionally to some highly overexpressing cells. To verify that our evaluation of the clustering efficiency, that is the GFP/mScarlet ratio, was not affected by the microscopy technique, we imaged cells with two calibrated microscopes (Total Internal Reflection Fluorescence (TIRF) microscopy and Spinning disk confocal (SDC) microscopy). As can be seen in f, all cells fall along the same line, suggesting a similar GFP/mScarlet ratio independently on the expression level or the microscopy technique. (overexpression imaged by spinning disk (SDC): n=12 cells; overexpression imaged by TIRF: n=15 cells; stable expression imaged by TIRF: n=−50 cells, this last dataset corresponds to FIG. 5F, reproduced here for convenience). By pooling all data together (g), we evaluated the median GFP/mScarlet ratio at 1.64 (n=14074 arrays in N=77 cells). dash lines: theoretical boundary GFP/mScarlet ratios for either a 1:1 BGFP: GBP-TM-mScarlet ratio, in case both GFPs of the BGFP dimer are bound to GBP, or a 2:1 ratio, in case only one GFP of the BGFP dimer is bound to GBP. (h) Evaluation of the A/B ratio in arrays polymerised on cells with BGFP and AmScarlet taking into account FRET between GFP and mScarlet (see methods; n=1058 arrays in N=12 cells). The ratio is nearly identical to the ideal 1:1 ratio suggesting that arrays made on cells have the same level of order as those made in vitro. (i) Measurement of the surface density of GBP-TM-mScarlet as a function of GBP-TM-mScarlet expression levels. Stable NIH3T3 cells expressing GBP-TM-mScarlet under Doxycycline (Dox)-inducible promoter where treated with increasing doses for Dox for 24 h, then briefly incubated with purified GFP and the amount of immobilized GFP per cell was assessed by flow cytometry (mean fluorescence per cell, n>4000 cells/sample).

FIG. 24 (a-g). Consequences of clustering on EGFR receptor endocytosis and Tie2 signalling. (a-b) Clustering of EGFR into a 3D spherical geometry does not induce endocytic block. (a) Endogenous EGF receptors (EGFR) on HeLa cells were clustered using GBP-EGFR-Darpin and either 3D icosahedral nanocages functionalized with GFP, or trimeric GFP unassembled building block as a control. After varying chase time, cells were fixed, processed for immunofluorescence with anti-LAMP1 antibodies and imaged by spinning disk confocal microscopy. Images correspond to single confocal planes, and side panels correspond to split-channel, high-magnification of the indicated regions. (b) Automated quantification of the colocalization between GFP and LAMP1 in the samples described in (a). n indicates number of cells analyzed per condition. Statistics were performed using an ANOVA1 test followed by Tukey's post-hoc test (p<0.01). There is very little (if any) endocytic block for EGF receptors clustered with the 60mer nanocages as the percentage of colocalization is similar between control GFP timers and GFP 60mer icosahedron. (c-f) Clustering of EGF receptors via arrays induces endocytic block. (c) Experiment scheme: Scrum starved HeLa cell were incubated with 20 ug/mL GBP-anti EGFR Darpin in DMEM-0.1% serum, then washed in DMEM-0.1% serum, then incubated with 0.5 μM B(c)GFP in DMEM-0.1% serum, then washed in DMEM-0.1% serum, then 0.5 μM A in DMEM-0.1% serum is added. Cells are then either imaged live (d) or incubated in DMEM-0.1% serum for 40 minutes before fixation and processing for immunofluorescence using anti-LAMP1 antibodies (f). (d) Addition of A induces rapid clustering of EGFR, in a similar fashion to the GBP-TM-mScarlet construct (see FIG. 5.a-b). (e) Automated quantification of the number of tracks of arrays as a function of time reveals that the dynamics of array formation is fast and quantitatively similar to the GBP-TM-mScarlet construct (compare with FIG. 5.c-d). (f) EGF receptors on HeLa cells were clustered (or not) as in a. Cells were then fixed and processed for immunofluorescence using LAMP1 antibodies and imaged by spinning disk confocal microscopy. After 40 min chase, unclustered EGFR extensively colocalizes with lysosomal marker LAMP1, while clustered EGFR stays at the plasma membrane, suggesting that array-induced 2D clustering of EGFR inhibits its endocytosis. Images correspond to maximum-intensity z-projections across entire cells (insets correspond to single confocal planes). Images correspond to split channels of FIG. 5.l. (g) Assembly of Tie2 cluster via on-cell assembly of arrays is as potent at inducing AKT signaling as preformed arrays. The A(c)Fd alone elicits much less AKT phosphorylation alone than when assembled into arrays by the B subunits on cells. Assembly here is done sequentially as in FIG. 5 by first incubating with A(c)Fd followed by extensive washing of unbound A(c)Fd, then by adding the B subunit. As a reference, cells were treated with preformed A(c)Fd/B arrays. Induction of phospho AKT is similar between A(c)Fd/B arrays assembled on cells or preassembled. Scale bars: 10 μm(a, d—left panel and k) and 1 μm (a, d insets and d—right panel).

### DETAILED DESCRIPTION

All references cited are herein incorporated by reference in their entirety. Within this application, unless otherwise stated, the techniques utilized may be found in any of several well-known references such as: Molecular Cloning; A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion. Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I), leucine (Leu; L), lysine (Lys; K), methionine (Met; M), phenylalanine (Phe; F), proline (Pro: P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising an amino acid sequence having at least 50%, 55%, 60%, 65%, 70%, 75%, 80%, 85%, 90%, 91%, 92%, 93%, 94%, 95%, 96%, 97%, 98%, or 99% sequence identity to the amino acid sequence of SEQ ID NO:1, wherein the polypeptide includes a mutation at 1, 2, 3, 4, 5, 6, 7, 8, 9, 10, 11, 12, 13, 14 or all 15 positions selected from the group consisting of T210, A213, Q215, Q216, Q217, Q219, K220, K222, A223, E224, F225, A226, Q227, Q229, and K230 relative to SEQ ID NO:1, wherein residues in parentheses are optional and may be present or absent.

1d2t
(SEQ ID NO: 1)
(LALVATGNDTTTKP)DLYYLKNSEAINSLALLPPPFAVGSIAFLNDQAM
YEQGRLLRNTERGKLAAEDANLSSGGVANAFSGAFGSPITEKDAPALHKL
LTNMIEDAGDLATRSAKDHYMRIRPFAFYGVSTCNTTEQDKLSKNGSYFS
GHTSIGWATALVLAEINPQRQNEILKRGYELGQSRVICGYHWQSDVDAAR
VVGSAVVATLHTNPAFQQQLQKAKAEFAQHQK

As described in the examples below, the polypeptides of this aspect are “first polypeptides” (also referred to as “A” components herein), capable of homo-oligomerization and interaction via a rigid interface with “second polypeptides” (or “B components) as defined below to produce the two-dimensional materials disclosed herein.

The polypeptide of SEQ ID NO:1 is not capable of such co-assembly; mutations at one or more of positions T210, A213, Q215, Q216, Q217, Q219, K220, K222, A223, E224, F225, A226, Q227, Q229, K230 result in such co-assembly properties.

In some embodiments described herein, the optional residues are present in the polypeptides and considered in determining percent identity relative to SEQ ID NO:1; in other embodiments, the optional residues are not present and are not considered in determining percent identity relative to SEQ ID NO:1.

In one embodiment, mutations in the polypeptide relative to SEQ ID NO:1 comprise:
(a) 1, 2, 3, 4, or all S of A213E, Q216A, Q219I, A223I, A226K;
(b) 1, 2, 3, 4, 5, 6, 7, 8, 9, 10, 11, or all 12 of Q215I, Q216A, Q217A, Q219I, K222L, A223I, E224L, F225T, A226H, Q227R, Q229R, K230T;
(c) 1, 2, 3, 4, 5, 6, 7, 8, 9, 10, I1, 12, or all 13 of T210R, Q215I, Q216A, Q217A, Q219I, K222L, A223I, E224L, F225T, A226Y, Q227R, Q229R, K230T;
(d) 1, 2, 3, 4, 5, 6, 7, 8, 9, 10, 11, 12, or all 13 of A213K, Q215I, Q216S, Q217A, Q219I, K222L, A223I, E224L, F225T, A226V, Q227R, Q229R, K230T;
(e) 1, 2, 3, 4, 5, 6, 7, 8, 9, 10, 11, 12, 13, or all 14 of T210R, A213K, Q215I, Q216S, Q217A, Q219I, K222L, A223I, E224L, F225T, A226Y, Q227R, Q229R, K230T;
(f) 1, 2, 3, 4, 5, 6, 7, 8, 9, 10, I1, 12, or all 13 of A213K, Q215I, Q216S, Q217A, Q219I, K222L, A223I, E224L, F225T, A226Y, Q227R, Q229R, K230T;
(g) 1, 2, 34, 5, 6, 7, 8, 9, 10, I1, 12, or all 13 of A213K, Q215I, Q216S, Q217A, Q219I, K222L, A223I, E224L, F225T, A226H, Q227R, Q229R, K230T;
(h) 1, 2, 3, 4, 5, 6, 7, 8, 9, 10, or all 11 of A213E, Q216A, Q219I, K222L, A223I, E224L, F225T, A226Y, Q227R, Q229R, K230T;
(i) 1, 2, 3, 4, 5, 6, 7, 8, 9, 10, or all 11 of A213E, Q216A, Q219I, K222L, A223I, E224L, F225T, A226H, Q227R, Q229R, K230T;
(j) 1, 2, 3, 4, 5, 6, 7, 8, 9, 10, 11, or all 12 of T210R, A213E, Q216A, Q219I, K222L, A223I, E224L, F225T, A226Y, Q227R, Q229R, K230T;
(k) 1, 2, 3, 4, 5, 6, 7, 8, 9, 10, 11, or all 12 of A213K, Q215I, Q216S, Q217A, Q219I, K220E, K222L, A223L, A226L, Q227E, Q229R, K230Q;
(l) 1, 2, 3, 4, 5, 6, 7, 8, 9, or all 10 of A213E, Q216A, Q219I, K220E, K222L, A223L, A226L, Q227E, Q229R, K230Q;
(m) 1, 2, 3, 4, 5, 6, or all 7 of Q215I, Q216A, Q217A, Q219I, A223I, E224L, Q227E;
(n) 1, 2, 3, 4, 5, 6, 7, or all 8 of A213K, Q215I, Q216S, Q217A, Q219I, A223I, E224L, Q227E;
(o) 1, 2, 3, 4, 5, 6, 7, or all 8 of A213K, Q215I, Q216S, Q217A, Q219I, A223I, E224L, Q227E;
(p) 1, 2, 3, 4, 5, or all 6 of A213D, Q217A, Q219I, K222L, A223L, Q227E;
(q) 1, 2, 3, 4, 5, 6, or all 7 of A213D, Q217A, Q219I, K222L, A223L, A226H, Q227E;
(r) 1, 2, 3, 4, 5, 6, 7, 8, or all 9 of A213K, Q215R, Q216S, Q217N, Q219I, K220R; A223I, A226K, Q227R
(s) 1, 2, 3, 4, 5, 6, 7, 8, or al 9 of A213K, Q215R, Q216S, Q217N, Q219I, K220R, A223I, A226T, Q227R;
(t) 1, 2, 3, 4, 5, 6, 7, or all 8 of A213K, Q215R, Q216S, Q217N, Q219I, K220R, A223I, Q227R;
(u) 1, 2, 3, 4, 5, 6, 7, 8, or all 9 of A213K, Q215R, Q216S, Q217N, Q219I, K220R, A223I, A226K, Q227R;
(v) 1, 2, 3, 4, 5, 6, 7, 8, or all 9 of A213E, Q215I, Q216S, Q217N, Q219I, K220E, A223I, A226V, Q227D; or
(w) 1, 2, 3, 4, 5, 6, 7, or all 8 of A213E, Q216V, Q219I, K220E, K222L, A223E, A226T, Q227E.

Each of these embodiments is present in a specific polypeptide disclosed herein capable of acting as a first polypeptide in the 2D materials disclosed herein.

In one embodiment, mutations in the polypeptide comprise mutations at 1, 2, 3, 4, 5, 6, 7, 8, 9, 10, 11, 12, 13, 14, 15, 16, or all 17 residues selected from residues 10, 65, 72, 73, 74, 77, 81, 85, 89, 90, 96, 100, 119, 152, 157, 167, and 197 relative to SEQ ID NO:1.

As disclosed in the examples, mutations at one or more of these positions lead to increased stability of the polypeptides. In a further embodiment, mutations in the polypeptide comprise 1, 2, 3, 4, 5, 6, 7, 8, 9, 10, 11, 12, 13, 14, 15, 16, or all 17 mutations selected from the group consisting of 10A, 65Q, 72P, 73E, 74Q or 74H, 77K, 81C, 85F, 89P, 90E, 96Y, 100R, 119Q, 152A, 157M or 157F, 167D, and 197G relative to SEQ ID NO:1.

In another embodiment, the polypeptide comprises an amino acid sequence having at least 50%, 55%, 60%, 65%, 70%, 75%, 80%, 85%, 90%, 91%, 92%, 93%, 94%, 95%, 96%, 97%, 9814N or 99% sequence identity to the amino acid sequence selected from the group consisting of SEQ ID NOS:2-31, wherein residues in parentheses may be present or absent.

AS1
(SEQ ID NO: 2)
(LALVATGNDTTTKP)DLYYLKNSEAINSLALLPPPPAVGSIAFLNDQAMYEQGRLLRNTERGKLA
AEDANLSPEGVANAFSCAFGSPITPKDAPALYKLLRNMIEDAGDLATRSAKDHYMRIRPFAFYGVS
TCNTTEQDKLSKNGSYPSGHTSIGWAFALVLAEINPQRQNEILKRGYELGQSRVICGYHWQSDVDA
ARVVGSAVVATLHTNPEFQAQLIKAKIEFKQHQK
(SEQ ID NO: 3)
(MGHHHHHHGGLALVATGNDTTTKP)DLYYLKNSEAINSLALLPPPPAVGSIAFLNDQAMYEQGRL
LRNTERGKLAAEDANLSPEGVANAFSCAFGSPITPKDAPALYKLLRNMIEDAGDLATRSAKDHYMR
IRPFAFYGVSTCNTTEQDKLSKNGSYPSGHTSIGWAFALVLAEINPQRQNEILKRGYELGQSRVIC
GYHWQSDVDAARVVGSAVVATLHTNPEFQAQLIKAKIEFKQHQK(EL)
As2
(SEQ ID NO: 4)
(LALVATGNDATTKP)DLYYLKNSEAINSLALLPPPPAVGSIAFLNDQAMYEQGRLLENTERGKLA
AEDANLSPEHVANAFSCAFGSPITPKDAPALYKLLRNMIEDAGDLATRSAKDHYMRIRPFAFYGVS
TCNTTEQDKLSKNGSYPSGHTSIGWAMALVLAEINPDRQNEILKRGYELGQSRVICGYHWQSDVDA
ARVVGSAVVATLHTNPEFQAQLIKAKIEFKQHQK
(SEQ ID NO: 5)
(MGHHHHHHGGLALVATGNDATTKP)DLYYLKNSEAINSLALLPPPPAVGSIAFINDQAMYEQGRL
LRNTERGKLAAEDANLSPEHVANAFSCAFGSPITPKDAPALYKLLRNMIEDAGDLATRSAKDHYMR
IRPFAFYGVSTCNTTEQDKLSKNGSYPSGHTSIGWAMALVLAEINPDRQNEILKRGYELGQSRVIC
GYHWQSDVDAARVVGSAVVATLHTNPEFQAQLIKAKIEFKQHQK (EL)
As3
(SEQ ID NO: 6)
(LALVATGNDATTKP)DLYYLKNSEAINSLALLPPPPAVGSIAFLNDQAMYEQGRLLRNTERGKLA
QEDANLSPEQVAKAFSCAFGFPITPEDAPALYKLLRNMIEDAGDLATRSAKDHYQRIRPFAFYGVS
TCNTTEQDKLSKNGSYPSGHTAIGWAFALVLAEINPDRQNEILKRGYELGQSRVICGYHWQSDVDA
GRVVGSAVVATLHTNPEFQAQLIKAKIEFKQHQK
(SEQ ID NO: 7)
(MGHHHHHHGGLALVATGNDATTKP)DLYYLKNSEAINSLALLPPPPAVGSIAFLNDQAMYEQGRL
LRNTERGKLAQEDANLSPEQVAKAFSCAFGFPITPEDAPALYKLLRNMIEDAGDLATRSAKDHYQR
IRPFAFYGVSTCNTTEQDKLSKNGSYPSGHTAIGWAFALVLAEINPDRQNEILKRGYELGQSRVIC
GYHWQSDVDAGRVVGSAVVATLHTNPEFQAQLIKAKIEFKQHQK (EL)
Di_13_0A
(SEQ ID NO: 8)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRFFAFYGVSTCNTQD
KLSKNGSYPSGHTSIGWATALVLAEINPQRQNEILKRGYELGQSRVICGYHWQSDVDAARVVGSAV
VATLHTNPEFQAQLIKAKIEFKQHQK
Di_13_1A
(SEQ ID NO: 9)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHTNPAFTAALIKALILTHRHRT
Di_13_2A
(SEQ ID NO: 10)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHRNPAFIAALIKALILTYRHRT
Di_13_3A
(SEQ ID NO: 11)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHTNPKFISALIKALILTVRHRT
Di_13_4A
(SEQ ID NO: 12)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHRNPKFISALIKALILTYRHRT
Di_13_5A
(SEQ ID NO: 13)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLINMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHTNPKFISALIKALILTYRHRT
Di_13_6A
(SEQ ID NO: 14)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATPSAKDHYMRIRPFAFYGVSTCNTQD
KLSKNGSYPSGHTSIGWATALVLAEINPQRQNEILKRGYELGQSRVICGYHWQSDVDAARVVGSAV
VATLHTNPKFISALIKALILTHRHRT
Di_13_7A
(SEQ ID NO: 15)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHTNPEFQAQLIKALILTYRHRT
Di_13_8A
(SEQ ID NO: 16)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHTNPEFQAQLIKALILTHRHRT
Di_13_9A
(SEQ ID NO: 17)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHRNPEFQAQLIKALILTYRHRT
Di_13_10A
(SEQ ID NO: 18)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSAVICGYHWQSDVDAARVVGSAV
VATLHTNPKFISALIEALLEFLEHRQ
Di_13_11A
(SEQ ID NO: 19)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKEGYELGQSRVICGYHWQSDVDAARVVGSAV
VATLHTNPEFQAQLIEALLEFLEHRQ
Di_13_12A
(SEQ ID NO: 20)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKEGYELGQSRVICGYHWQSDVDAARVVGSAV
VATLHTNPAFIAALIKAKILFAEHQK
Di_13_13A
(SEQ ID NO: 21)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYFSGHTSIGWATALVLAEINPQRQNEILKRGYELGQSRVICGYHWQSDVDAARVVGSAV
VATLHTNPKFISALIKAKILFAEHQK
Di_13_14A
(SEQ ID NO: 22)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHTNPKFISALIKAKILFAEHQK
Di_13_15A
(SEQ ID NO: 23)
(GNDTTTKP)DLYYLKNSEAINSLALLPPPPAVGSIAFLNDQAMYEQGRLLRNTERGKLAAEDANL
SSSGVANAFSGAFGSPITEKDAPALHKLLTNMIEDAGDLATRSAKDHYMRIRPFAFYGVSTCNTQD
KLSKNGSYPSGHTSIGWATALVIAEINPQRQNEILKRGYELGQSRVICGTHWQSDVDAARVVGSAV
VATLHTNPDFQQALIKALLEFAEHQK
Di_13_16A
(SEQ ID NO: 24)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSANDHYMRIPPFAFYGVSTCNTQD
KLSKNGSYPSGHTSIGWATALVLAEINPQRQNEILKRGYELGQSRVICGYHWQSDVDAARVVGSAV
VATLHTNPDFQQALIKALLEFHEHQK
Di_13_17A
(SEQ ID NO: 25)
(GNDTTTKP)DLYYLKNSEAINSLALLPPPPAVGSIAFINDOAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGVHWQSDVDAARVVGSAV
VATLHTNPKFRSNLIRAKIEFKRHQK
Di_13_18A
(SEQ ID NO: 26)
(GNDTTTKF)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHTNPKFRSNLIRAKIEFTRHQK
Di_13_19A
(SEQ ID NO: 27)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHTNPKFRSNLIRAKIEFARHQK
Di_13_20A
(SEQ ID NO: 28)
(GNDTTTKP)DLYYLKNSEAINSLALLPPPPAVGSIAFLNDQAMYEQGRLLRNTERGKLAAEDANL
ZSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHTNPKFRSNLIRAKIEFKRHQK
Di_13_21A
(SEQ ID NO: 29)
(GNDTTTKP)DLYYLKNSEAINSLALLPPPPAVGSIAFLNDQAMYEQGRLLRNTERGKLAAEDANL
SSGGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAV
VATLHTNPEFISNLIEAKIEFVDHQK
Di_13_22A
(SEQ ID NO: 30)
(GNDTTTKP)DLYYLKNSEAINSLALLPPPPAVGSIAFLNDQAMYEQGRLLRNTERGKLAAEDANL
SSSGVANAFSGAFGSPITEKDAPALHKLLTNMIEDAGDLATRSAKDHYMRIRPFAFYGVSTCNTQD
KLSKNGSYPSGHTSIGWATALVLAEINPQRQNEILKRGYELGQSRVICGYHWQSDVDAARVVGSAY
VATLHTNPEFQVQLIEALEEFTEHQK
cyclic A comp. with N-term spyCatcher fusion for peptide fusion
(SEQ ID NO: 31)
(MGHHHHHH)SGAMVDTLSGLSSEQGQSGDMTTEEDSATHIKFSKRDEDGKELAGATMELRDSSGK
TISTWISDGQVRDFYLYPGKYTFVETAAPDGYEVATAITFTVNEQGQVTVNGKATKGDAHIGGSGG
SGGLALVATGNDTTTKPDLYYLKNSEAINSLALLPPPPAVGSIAFLNDQAMYEQGRLLRNTERGKL
AAEDANLSSGGVANAFSGAFGSPITEKDAPALHKLLTNMIEDAGDLATRSAKDHYMRIRPFAFYGV
STCNTTEQDKLSKNGSYPSGHTSIGWATALVLAEINPQRQNEILKRGYELGQSRVICGYHWQSDVD
AARVVGSAVVATLHTNPEFQAQLIKAKIEFKQHQKFRQQPPPPQQSGGNDATTKPDLYYLKNSEAI
NSLALLPPPPAVGSIAFLNDQAMYEQGRLLRNTERGKLAQEDANLSPEQVAKAFSCAFGFPITPED
APALYKLLRNMIEDAGDLATRSAKDHYQRIRPFAFYGVSTCNTTEQDKLSKNGSYPSGHTAIGWAF
ALVLAEINPDRQNEILKRGYELGQSRVICGYHWQSDVDAGRVVGSAVVATLHTNPEFQAQLIKAKI
EFKQHQK

In all polypeptide embodiments for all aspects of the disclosure, in one embodiment the optional residues are present in the polypeptides and considered in determining percent identity relative to the reference sequence; in other embodiments, the optional residues are not present and are not considered in determining percent identity relative to the reference sequence.

In one embodiment, underlined residues of the polypeptide are conserved relative to the reference amino acid sequence. In these embodiments, the underlined residues comprise the region of the polypeptide involved in forming a rigid interface of 2D protein materials when homo-oligomers of these “first” polypeptides co-assemble with homo-oligomers of the “second” polypeptides, embodiments of which are described below.

The polypeptides of this first aspect may comprise one or more additional functional peptide domains. Any functional domain may be added as deemed appropriate for an intended use. Exemplary embodiments of such fusion proteins include, but are not limited to, polypeptides having at least 50%, 55%, 60%, 65%, 70%, 75%, 80%, 85%, 90%, 91%, 92%, 93%, 94%, 95%, 96%, 97%, 98%, or 99% sequence identity to the amino acid sequence of a sequence below, wherein residues in parentheses are optional and may be present or absent.

A comp. N-term fusion (Di13A_NHISTEV_mCherryEm)
(SEQ ID NO: 32)
(MGHHHHHH)GENLYFQGVSKGEAVIKEFMRFKVHMEGSMNGHEFEIEGEGEGRPYEGTQTAKLKVTK
GGPLPFSWDILSPQFMYGSRAFTKHPADIPDYYKQSFPEGFKWERVMNFEDGGAVTVTQDTSLEDGTL
IYKVKLRGTNFPPDGPVMQKKTMGWEASTERLYPEDGVLKGDIKMALRLKDGGRYLADFKTTYKAKKP
VQMPGAYNVDRKLDITSHNEDYTVVEQYERSEGRHSTGGMDELYKGSGLALVATGNDTTTKPDLYYLK
NSEAINSLALLPPPPAVGSIAFLNDQAMYEQGRLLRNTERGKLAAEDANLSSGGVANAFSGAFGSPIT
EKDAPALHKLLTNMIEDAGDLATRSAKDHYMRIRPFAFYGVSTCNTTEQDKLSKNGSYPSGHTSIGWA
TALVLAEINPQRQNEILKRGYELGQSPVICGYHWQSDVDAARVVGSAVVATLHTNPEFQAQLIKAKIE
FKQHQK
A comp. N-term fusion (Di13A_NHisTEV_mKate2)
(SEQ ID NO: 33)
(MGHHHHHH)GENLYFQGMVSELIKENMEMKLYMEGTVNNHHFKCTSEGEGKPYEGTQTMRIKAVEGG
PLPFAFDILATSFMYGSKTFINHTQGIPDFFKQSFPEGFTWERVTTYEDGGVLTATQDTSLQDGCLIY
NVKIRGVNFPSNGPVMQKKTLGWEASTETLYPADGGLEGRADMALKLVGGGHLICNLKTTYRSKKPAK
NLKMPGVYYVDRRLERIKEADKETYVEQHEVAVARYCDLPSKLGHRGSGLALVATGNDTTTKPDLYYL
KNSEAINSLALLFPPPAVGSIAFLNDQAMYEQGRLLRNTERGKLAAEDANLSSGGVANAFSGAFGSPI
TEKDAPALHKLLTNMIEDAGDLATRSAKDHYMRIRPFAFYGVSTCNTTEQDKLSKNGSYPSGHTSIGW
ATALVLAEINPQRQNEILKRGYELGQSRVICGYHWQSDVDAARVVGSAVVATLHTNPEFQAQLIKAKI
EFKQHQK
A. comp. N-term fusion (Di13A_NHisTEV_mKO2)
(SEQ ID NO: 34)
(MGHHHHHH)GENLYFQGVSVIKPEMKMRYYMDGSVNGHEFTIEGEGTGRPYEGHQEMTLRVTMAEGG
PMPFAFDLVSHVFCYGHRVFTKYPEEIPDYFKQAFPEGLSWERSLEFEDGGSASVSAHISLRGNTFYH
KSKFTGVNFPADGPIMQNQSVDWEPSTEKITASDGVLKGDVTMYLKLEGGGNHKCQMKTTYKAAKEIL
EMPGDHYIGHRLVRKTEGNITEQVEDAVAHSGSGLALVATGNDTTTKPDLYYLKNSEAINSLALLPPP
PAVGSIAFLNDQAMYEQGRLLRNTERGKLAAEDANLSSGGVANAFSGAFGSPITEKDAPALHKLLTNM
IEDAGDLATRSAKDHYMRIRPFAFYGVSTCNTTEQDKLSKNGSYPSGHTSIGWATALVLAEINPQRQN
EILKRGYELGQSRVICGYHWQSDVDAARVVGSAVVATLHTNPEFQAQLIKAKIEFKQHQK
A comp. N-term fusion (Di13A_NHisTEV_mTagBFP2)
(SEQ ID NO: 35)
(MGHHHHHH)GENLYFQGMVSKGEELIKENMHMKLYMEGTVDNHHFKCTSEGEGKPYEGTQTMRIKVV
EGGPLPFAFDILATSFLYGSKTFINHTQGIPDFFKQSFPEGFTWERVTTYEDGGVLTATQDTSLQDGC
LIYNVKIRGVNFTSNGPVMQKKTLGWEAFTETLYPADGGLEGRNDMALKLVGGSHLIANAKTTYRSKK
PAKNLKMPGVYYVDYRLERIKEANNETYVEQHEVAVARYCDLPSKLGHKLNGSGLALVATGNDTTTKP
DLYYLKNSEAINSLALLPPPPAVGSIAFLNDQAMYEQGRLLRNTERGKLAAEDANLSSGGVANAFSGA
FGSPITEKDAPALHKLLTNMIEDAGDLATRSAKDHYMRIRPFAFYGVSTCNTTEQDKLSKNGSYPSGH
TSIGWATALVLAEINPQRQNEILKRGYELGQSRVICGYHWQSDVDAARVVGSAVVATLHTNPEFQAQL
IKAKIEFKQHQK
A comp. N-term fusion (Di13A_NHisTEV_mTurq2_sf)
(SEQ ID NO: 36)
(MGHHHHHH)GENLYFQGSKGEELFTGVVPILVELDGDVNGHKFSVRGEGEGDATNGKLTLKFICTTG
KLPVPWPTLVTTLSWGVQCFARYPDHMKQHDFFKSAMPEGYVQERTISFKDDGTYKTRAEVKFEGDTL
VNRIELKGIDFKEDGNILGHKLEYNYFSDNVYITADKQKNGIKANFKIRHNVEDGSVQLADHYQQNTP
IGDGPVLLPDNHYLSTQSVLSKDPNEKRDHMVLLEFVTAAGITHGMDELYGSGLALVATGNDTTTKPD
LYYLKNSEAINSLALLPPPPAVGSIAFLNDQAMYEQGRLLRNTERGKLAAEDANLSSGGVANAFSGAF
GSPITEKDAPALHKLLTNMIEDAGDLATRSAKDHYMRIRPFAFYGVSTCNTTEQDKLSKNGSYPSGHT
SIGWATALVLAEINPQRQNEILKEGYELGQSRVICGYHWQSDVDAARVVGSAVVATLHTNFEFQAQLI
KAKIEFKQHQK
A comp. N-term fusion (Di13A_NHisTEV_RFP)
(SEQ ID NO: 37)
(MGHHHHHH)GENLYFQGELIKENMHMKLYMEGTVNNHHFKATSEGEGKPYEGTQTMRIKVVEGGPLP
FAFDILATSFMYGSRTFIKHTQGIPDFFKQSFPEGFTWERVTTYEDGGVLTATQDTSLQDGMLIYNVK
IRGVNFPSNGPVMQKKTLGWEANTEMLYPADGGLEGRSDMALKLVGGGHLIVNFKTTYRSKKPAKNLK
MPGVYYVDHRLERIKEADKETYVEQHEVAVARYGSGLALVATGNDTTTKPDLYYLKNSEAINSLALLP
PPPAVGSIAFLNDQAMYEQGRLLRNTERGKLAAEDANLSSGGVANAFSGAFGSPITEKDAPALHKLLT
NMIEDAGDLATRSAKDHYMRIRPFAFYGVSTCNTTEQDKLSKNGSYPSGHTSIGWATALVLAEINPQR
QNEILKRGYELGQSRVICGYHWQSDVDAARVVGSAVVATLHTNPEFQAQLIKAKIEFKQHQK
A comp. N-term fusion (Di13A_NHisTEV_SYFP2_sf)
(SEQ ID NO: 38)
(MGMHHHHH)GENLYFQGSKGEELFTGVVPILVELDGDVNGHKFSVRGEGEGDATNGKLTLKLICTTG
KLPVPWPTLVTTLGYGVQCFARYPDHMKQHDFFKSAMPEGYVQERTISFKDDGTYKTRAEVKFEGDTL
VNRIELKGIDFKEDGNILGHKLEYNYNSHNVYITADKQKNGIKANFKIRHNVEDGSVQLADHYQQNTP
IGDGPVLLPDNHYLSYQSVLSKDPNEKRDHMVLLEFVTAAGITHGMDELYGSGLALVATGNDTTTKPD
LYYLKNSEAINSLALLPPPPAVGSIAFLNDQAMYEQGRLLRNTERGKLAAEDANLSSGGVANAFSGAF
SIGWATALVLAEINPQRQNEILKEGYELGQSRVICGYHWQSDVDAARVVGSAVVATLHTNPEFQAQLI
KAKIEFKQHQK
A comp. with N-term spyCatcher fusion for peptide fusion
(Di_13_A_2_N_SC)
(SEQ ID NO: 39)
(MGHHHHHH)SGAMVDTLSGLSSEQGQSGDMTIEEDSATHIKFSKRDEDGKELAGATMELRDSSGKTI
STWISDGQVKDFYLYFGKYTFVETAAPDGYEVATAITFTVNEQGQVTVNGKATKGDAHIGGSGGSGGN
DTTTKPDLYYLKNSEAINSLALLPPPPAVGSIAFLNDQAMYEQGRLLRNTERGKLAAEDANLSSGGVA
NAFSGAFGSPITEKDAPALHKLLTNMIEDAGDLATRSAKDHYMRIRPFAFYGVSTCNTTEQDKLSKNG
SYPSGHTSIGWATALVLAEINPQRQNEILKRGYELGQSRVICGYHWQSDVDAARVVGSAVVATLHTNP
EFQAQLIKAKIEFKQHQK
A comp. N-term fusion (Di_13_A_GFP_N)
(SEQ ID NO: 40)
(MGHHHHHH)GSSKGEELFTGVVPILVELDGDVNGHKFSVRGEGEGDATNGKLTLKFICTTGKLPVPW
PTLVTTLTYGVQCFARYPDHMKQHDFFKSAMPEGYVQERTISFKDDGTYKTRAEVKFEGDTLVNRIEL
KGIDFKEDGNILGHKLEYNFNSHNVYITADKQKNGIKANFKIRHNVEDGSVQLADHYQQNTPIGDGPV
LLPDNHYLSTQSVLSKDPNEKRDHMVLLEFVTAAGITHGMDELYMGLALVATGNDTTTKPDLYYLKNS
EAINSLALLPPPPAVGSIAFLNDQAMYEQGRLLRNTERGKLAAEDANLSSGGVANAFSGAFGSPITEK
DAPALHKLLTNMIEDAGDLATRSAKDHYMRIRPFAFYGVSTCNTTEQDKLSKNGSYPSGHTSIGWATA
LVLAEINPQRQNEILKRGYELGQSRVICGYHWQSDVDAARVVGSAVVATLHTNPEFQAQLIKAKIEFK
QHQK
A comp. N-term fusion with a short linker (Di_13_A_GFP_N_loop6)
(SEQ ID NO: 41)
(MGHHHHHH)GSSKGEELFTGVVPILVELDGDVNGHKFSVRGEGEGDATNGKLTLKFICTTGKLPVPW
PTLVTTLTYGVQCFARYPDHMKQHDFFKSAMPEGYVQERTISFKDDGTYKTRAEVKFEGDTLVNRIEL
KGIDFKEDGNILGHKLEYNFNSHNVYITADKQKNGIKANFKIRHNVEDGSVQLADHYQQNTPIGDGPV
LLPDNHYLSTQSVLSKDPNEKRDHMVLLEFVTAAGITHGSGKPDLYYLKNSEAINSLALLPPPPAVGS
IAFLNDQAMYEQGRLLRNTERGKLAAEDANLSSGGVANAFSGAFGSPITEKDAPALHKLLTNMIEDAG
DLATRSAKDHYMRIRPFAFYGVSTCNTTEQDKLSKNGSYPSGHTSIGWATALVLAEINPQRQNEILKR
GYELGQSRVICGYHWQSDVDAARVVGSAVVATLHTNPEFQAQLIKAKIEFKQHQK
A comp. N-term fusion of GFP and AVI tag. (Di_13_A_N_AVI_GFP)
(SEQ ID NO: 42)
(MGHHHHHH)GSENLYFQGSGGLNDIFEAQKIEWHESKGEELFTGVVPILVELDGDVNGHKFSVRGEG
EGDATNGKLTLKFICTTGKLPVPWPTLVTTLTYGVQCFARYPDBMKQHDFFKSAMPEGYVQERTISFK
DDGTYKTRAEVKFEGDTLVNRIELKGIDFKEDGNILGHKLEYNFNSHNVYITADKQKNGIKANFKIRH
NVEDGSVQLADHYQQNTPIGDGPVLLPDNHYLSTQSVLSKDPNEKRDHMVLLEFVTAAGITHGMDELY
MGLALVATGNDTTTKPDLYYLKNSEAINSLALLPPPPAVGSIAFLNDQAMYEQGRLLRNTERGKLAAE
DANLSSGGVANAFSGAFGSPITEKDAPALHKLLTNMIEDAGDLATRSAKDHYMRIRPFAFYGVSTCNT
TEQDKLSKNGSYPSGHTSIGWATALVLAEINPQRQNEILKEGYELGQSRVICGYHWQSDVDAARVVGS
AVVATLHTNPEFQAQLIKAKIEFKQHQK
A comp. N-term fusion (Di_13_A_N_mEOS4)
(SEQ ID NO: 43)
(MGHHHHHH)MVSAIKPDMRIKLRMEGNVNGHHFVIDGDGTGKPYEGKQTMDLEVKEGGPLPFAFDIL
TTAFHYGNRVFVKYPDNIQDYFKQSFPKGYSWERSLTFEDGGICYARNDITMEGDTFYNKVRFYGTNF
PANGPVMQKKTLKWEPSTEKMYVRDGVLTGDIHMALLLEGNAHYRCDFRTTYKAKEKGVKLPGYHFVD
HAIEILSHDKDYNKVKLYEHAVAHSGLPDNARRSGLALVATGNDTTTKPDLYYLKNSEAINSLALLPP
PPAVGSIAFLNDQAMYEQGRLLRNTERGKLAAEDANLSSGGVANAFSGAFGSPITEKDAPALHKLLTN
MIEDAGDLATRSAKDHYMRIRPFAFYGVSTCNTTEQDKLSKNGSYPSGHTSIGWATALVLAEINPQRQ
NEILKRGYELGQSRVICGYHWQSDVDAARVVGSAVVATLHTNPEFQAQLIKARIEFKQHQK
A comp. N-term fusion for peptide fusion (Di_13_A_N_SNAPtag)
(SEQ ID NO: 44)
(MGHHHHHH)MDKDCEMKRTTLDSPLGKLELSGCEQGLHRIIFLGKGTSAADAVEVPAPAAVLGGPEP
LMQATAWLNAYFHQPEAIEEFPVPALHHPVFQQESFTRQVLWKLLKVVKFGEVISYSHLAALAGNPAA
TAAVKTALSGNPVPILIPCHRVVQGDLDVGGYEGGLAVKEWLLAHEGHRLGKPGLGGLALVATGNDTT
TKPDLYYLKNSEAINSLALLPPPPAVGSIAFLNDQAMYEQGRLLRNTERGKLAAEDANLSSGGVANAF
SGAFGSPITEKDAPALHKLLTNMIEDAGDLATRSAKDHYMRIRPFAFYGVSTCNTTEQDKLSKNGSYP
SGHTSIGWATALVLAEINPQRQNEILKRGYELGQSRVICGYHWQSDVDAARVVGSAVVATLHTNPEFQ
AQLIKAKIEFKQHQK
A comp. N-term fusion for peptide fusion (Di_13_A_N_spyTag)
(SEQ ID NO: 45)
(MGHHHHHH)GSENLYFQGSGHMKPLRGAVFSLQKQHPDYPDIYGAIDQNGTYQNVRTGEDGKLTFKN
LSDGKYRLFENSEPAGYKPVQNKPIVAFQIVNGEVRDVTSIVPQDIPATYEFTNGKHYITNEPIPPKG
LALVATGNDTTTKPDLYYLKNSEAINSLALLPPPPAVGSIAFLNDQAMYEQGRLLRNTERGKLAAEDA
NLSSGGVANAFSGAFGSPITEKDAPALHKLLTNMIEDAGDLATRSAKDHYMRIRPFAFYGVSTCNTTE
QDKLSKNGSYPSGHTSIGWATALVLAEINPQRQNEILKRGYELGQSRVICGYNWQSDVDAARVVGSAV
VATLHTNPEFQAQLIKAKIEFKQHQK
A_n_mScarlet
A comp. N-term fusion
(SEQ ID NO: 46)
NKIHGS(hhhhhh)GSTSGSAMVSKGEAVIKEFMRFKVHMEGSMNGHEFEIEGEGEGRPYEGTQTAKL
KVTKGGPLPFSWDILSPQFMYGSRAFTKHPADIPDYYKQSFPEGFKWERVMNFEDGGAVTVTQDTSLE
DGTLIYKVKLRGTNFFPDGPVMQKKTMGWEASTERLYPEDGVLKGDIKMALRLKDGGRYLADFKTTYK
AKKPVQMPGAYNVDRKLDITSHNEDYTVVEQYERSEGRHSTGGMDELYKGGSGGSLALVATGNDTTTK
PDLYYLKNSEAINSLALLPPPPAVGSIAFLNDQAMYEQGRLLRNTERGKLAAEDANLSSGGVANAFSG
AFGSPITEKDAPALHKLLTNMIEDAGDLATRSAKDHYMRIRPFAFYGVSTCNTTEQDKLSKNGSYPSG
HTSIGWATALVLAEINPQRQNEILKRGYELGQSRVICGYHWQSDVDAARVVGSAVVATLHTNPEFQAQ
LIKAKIEFKQHQK

### A(d) Fused to SpyCatcher and Alpha

>Di13ALIAs3_380Loop_SC_nAlpha
(SEQ ID NO: 47)
(MGHHHHHH)SGSGENLYFQGSGPSRLEELRRRLTEPGSLALVATGNDTT
TKPDLYYLKNSEAINSLALLPPPPAVGSIAFLNDQAMYEQGRLLRNTERG
KLAAEDANLSSGGVANAFSGAFGSPITEKDAPALHKLLTNMIEDAGDLAT
RSAKDHYMRIRPFAFYGVSTCNTTEQDKLSKNGSYPSGHTSIGWATALVL
AEINPQRQNEILKRGYELGQSRVICGYHWQSDVDAARVVGSAVVATLHTN
PEFQAQLIKAKIEFKQHQKFRQTGSGSGGAMVDTLSGLSSEQGQSGDMTI
EEDSATHIKFSKRDEDGKELAGATMELRDSSGKTISTWISDGQVKDFYLY
PGKYTFVETAAPDGYEVATAITFTVNEQGQVTVNGKATKGDAHIGSGSGS
GSGQDKLSKNGSYPSCHTAIGWAFALVLAEINPDRQNEILKRGYELGQSR
VICGYHWQSDVDAGRVVGSAVVATLHTNPEFQAQLIKAKIEFKQNQK

In one embodiment the optional residues are present in the polypeptides and considered in determining percent identity relative to the reference sequence; in other embodiments, the optional residues are not present and are not considered in determining percent identity relative to the reference sequence.

As discussed in detail herein, the polypeptides of this first aspect self-assemble into homo-oligomers comprising a first interface region that can interact with a second interface region of homo-oligomers of the polypeptides of the second aspect of the disclosure. Thus, in another embodiment, the disclosure provides homo-oligomers of the polypeptide of any embodiment or combination of embodiments of the first aspect of the disclosure.

In one embodiment, the homo-oligomer is a cyclic homo-oligomer. In a further embodiment, the cyclic homo-oligomer comprises a homo-oligomer of a polypeptide comprising an amino acid sequence having at least 50%, 55%, 60%, 65%, 70%, 75%, 80%, 85%, 90%, 91%, 92%, 93%, 94%, 95%, 96%, 97%, 98%, or 99% sequence identity to the amino acid sequence of SEQ ID NO:31, wherein residues in parentheses are optional. In one embodiment the optional residues are present in the polypeptides and considered in determining percent identity relative to the reference sequence; in other embodiments, the optional residues are not present and are not considered in determining percent identity relative to the reference sequence.

In a second aspect, the disclosure provides polypeptides comprising an amino acid sequence having at least 50%, 55%, 60%, 65%, 70%, 75%, 80%, 85%, 90%, 91%, 92%, 93%, 94%, 95%, 96%, 97%, 98%, or 99% sequence identity to the amino acid sequence of SEQ ID NO:100, wherein the polypeptide includes a mutation at 1, 2, 3, 4, 5, 6, 7, 8, 9, 10, 11, 12, 13, or all 14 positions selected from the group consisting of M1, N5, E8, K9, Q12, E13, H14, K16, I17, V18, Q19, A20, E22, and I23 relative to SEQ ID NO:100.

1tk9
(SEQ ID NO: 100)
MSIINLVEKEWQEHQKIVQASEILKGQIAKVGELLCECLKKGGKILICGN
GGSAADAQHFAAELSGRYKKERKALAGIALTTDTSALSAIGNDYGFEFVF
SRQVEALGNEKDVLIGISTSGKSPNVLEALKKAKELNMLCLGLSGKGGGM
MNKLCDHNLVVPSDDTARIQEMHILIIHTLCQIIDESF

As described herein, the polypeptides of this aspect are “second polypeptides” (also referred to as “B” components herein), capable of homo-oligomerization and interaction via a rigid interface with “first polypeptides” (or “A” components), which are defined above. The polypeptide of SEQ ID NO:100 is not capable of such co-assembly; mutations at one or more of positions M1, N5, ER, K9, Q12, E13, H14, K16, I17, V18, Q19, A20, E22, and I23 result in such co-assembly properties.

In one embodiment, mutations in the polypeptide relative to SEQ ID NO:100 comprise:
(a) 1, 2, 3, 4, 5, or all 6 of N5T, K9L, Q12L, K16L, A20L, I23R;
(b) 1, 2, 3, 4, 5, 6, 7, 8, 9, 10, 11, 12, or all 13 of M1S, N5A, E8H, K9L, Q12L, H14A, K16L, I17A, V18T, Q19V, A20L, E22S, I23S;
(c) 1, 2, 3, 4, 5, 6, 7, 8, 9, 10, 11, 12, or all 13 of M1S, N5A, E8Y, K9L, Q12L, H14A, K6L, I17A, V18T, Q19V, A20L, E22S, I23S;
(d) 1, 2, 3, 4, 5, 6, 7, 8, 9, 10, 11, or all 12 of M1S, N5A, K9L, Q12L, H14A, K16L, 117A. V18T, Q19V, A20N, E22S, I23D;
(e) 1, 2, 3, 4, 5, 6, 7, 8, 9, 10, 11, 12, or all 13 of M1S, N5A, E8Y, K9L, Q12L. H14A, K16L, I17A, V18T, Q19V, A20N, E22S, I23D;
(g) 1, 2, 3, 4, 5, 6, 7, 8, 9, 10, 11, 12, or all 13 of M1S, N5A, E8H, K9L, Q12L, H14A, K16L, I17A, V18T, Q19V, A20N, E22S, I23D;
(h) 1, 2, 34, 5, 6, 7, 8, 9, 10, 11, or all 12 of M1S, N5A, E8Y, K9L, Q12L, H14A, K16L, I17A, V18T, A20L, E22S, I23R;
(i) 1, 2, 3, 4, 5, 6, 7, 8, 9, 10, 11, or all 12 of M1S, N5A, E8H, K9L, Q12L, H14A, K16L, I17A, V18T, A20L, E22S, I23R;
(k) 1, 2, 3, 4, 5, 6, 7, 8, 9, or all 10 of M1S, N5T, K9R, Q12I, E13R, K6L, I17A, A20N, E22S, I23D;
(l) 1, 2, 3, 4, 5, 6, 7, 8, 9, or all 10 of M1S, N5T, K9R, Q12I, E3R, K16L, 117A, A20L, E22S, I23R;
(m) 1, 2, 3, 4, 5, 6, 7, 8, 9, 10, or all I1 of M1S, N5A, K9L, Q12L, E13R, K16L, 117A, Q19V, A20L, E22S, I23S;
(n) 1, 2, 3, 4, 5, 6, 7, 8, 9, 10, or all 11 of M1S, N5A, K9L, Q12L, E13R, K16L, I17A, Q19V, A20D, E22S, I23D;
(o) 1, 2, 3, 4, 5, 6, 7, 8, 9, 10, or all 11 of M1S, N5A, K9L, Q12L, E13R, K16L, I17A, Q19V, A20N, E22S, I23D;
(p) 1, 2, 3, 4, 5, 6, 7, or all 8 of M1A, N5Q. K9L, Q12I, E13K, K16L, E22A, I23R;
(q) 1, 2, 3, 4, 5, 6, 7, 8, or all 9 of M1A, N5Q, E8H, K9L, Q12I, E13K, K16L, E22A, I23R;
(r) 1, 2, 3, 4, 5, 6, 7, 8, 9, 10, or all I1 of M1S, N5T, K9L, Q12L, E13A, K16L, 117A, Q19E, A20L, E22S, I23S;
(s) 1, 2, 3, 4, 5, 6, 7, 8, 9, or all 10 of M1S, N5T, K9E, Q12L, K16L, I17A, Q19E, A20L, E22S, I23S;
(t) 1, 2, 3, 4, 5, 6, 7, 8, 9, 10, or all 11 of M1S, N5T, K9E, Q12L, E13A, K16L, I17A, Q19E, A20D, E22S, I23S;
(u) 1, 2, 3, 4, 5, 6, 7, 8, 9, 10, or all 11 of M1S, N5T, K9E, Q12L, E13A, K16L, 117A, Q19E, A20N, E22S, I23S;
(v) 1, 2, 3, 4, 5, 6, 7, 8, or all 9 of M1K, N5Q, Q12L, E13K, K16L, 117A, Q19V, A20R, I23R; or
(w) 1, 2, 3, 4, 5, 6, 7, 8, 9, or all 10 of M1A, N5Q, K9L, Q12I, E13K, K16L, 117A, A20R, E22A, I23R.

Each of these embodiments is present in a specific polypeptide disclosed herein capable of acting as a second polypeptide in the 2D materials disclosed herein.

In a further embodiment, mutations in the polypeptide comprise mutations at 1, 2, 3, 4, 5, 6, 7, 8, 9, 10, 11, 12, 13 or all 14 residues selected from residues 37, 38, 41, 98, 101, 111, 134, 137, 141, 150, 153, 158, 187, 189, and 190 relative to SEQ ID NO:100.

As disclosed in the attached appendices, mutations at one or more of these positions lead to increased stability of the polypeptides. In one embodiment, mutations in the polypeptide comprise 1, 2, 3, 4, 5, 6, 7, 8, 9, 10, 11, 12, 13, or all 14 residues selected from residues 37R, 38A, 41N, 98Y, 101A, 111G, 134R, 137G, 141I, 150K, 153D, 158C, 187A, 189E, and 190L relative to SEQ ID NO:100.

In a further embodiment, the polypeptide comprises an amino acid sequence having at least 50%, 55%, 60%, 65%, 70%, 75%, 80%, 85%, 90%, 91%, 92%, 93%, 94%, 95%, 96%, 97%, 98%, or 99% sequence identity to the amino acid sequence selected from the group consisting of SEQ ID NOS:50-82, wherein residues in parentheses may be present or absent. In one embodiment the optional residues are present in the polypeptides and considered in determining percent identity relative to the reference sequence; in other embodiments, the optional residues are not present and are not considered in determining percent identity relative to the reference sequence.

B
(SEQ ID NO: 50)
(MG)SLITLVELEWLEHQLIVQLSERLKGQTAKVGELLCECLKKGGKILICGNGGSAADAQHFAAELS
GRYKKERKALAGIALTTDTSALSAIGNDYGFEFVFSRQVEALGNEKDVLIGISTSGKSPNVLEALKKA
KELNMLCLGLSGKGGGMMNKLCDHNLVVPSDDTARIQEMHILIIHTLCQIIDESF(LEHHHHHH)
SEQ ID NO: 51)
SLITLVELEWLEHQLIVQLSERLKGQIAKVGELLCECLKKGGKILICGNGGSAADAQHFAAELSGRYK
KERKALAGIALTTDTSALSAIGNDYGFEFVFSRQVEALGNEKDVLIGISTSGKSPNVLEALKKAKELN
MLCLGLSGKGGGMMNKLCDHNLVVPSDDTARIQEMHILIIHTLCQIIDESF
B1
(SEQ ID NO: 52)
(MG)SLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
KELNMLCLGLSGKGGGKMNKLCDHNLVVPSDDTARIQEMHILIIHTLCQIIDEAF(LEHHHHHH)
(SEQ ID NO: 53)
SLITLVELEWLEHQLIVQLSERLKGQIAKVGELLCECLKNGGKILICGNGGSAADAQHFAAELSGRYK
KERKALAGIALTTDTSALSAIGNDYGFEFVFSRQVEALGNEGDVLIGISTSGKSPNVLEALKKAKELN
MLCLGLSGKGGGKMNKLCDHNLVVPSDDTARIQEMHILIIHTLCQIIDEAF
B2
(SEQ ID NO: 54)
(MG)SLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAF(LEHHHHHH)
(SEQ ID NO: 55)
SLITLVELEWLEHQLIVQLSERLKGQIAKVGELLCECLKNGGKILICGNGGSAADAQHFAAELSGRYK
KERKALAGIALTTDTSALSAIGNDYGFEFVFSRQVEALGNEGDVLIGISTSGKSPNVLEALKKARELN
MLCIGLSGKGGGKMNDLCDHNLVVPSDDTARIQEMHILIIHTLCQIIDEAF
B3
(SEQ ID NO: 56)
(MG)SLITLVELEWLEHQLIVQLSERLKGQIAKVGELLCRALKNGGKILICGNGGSAADAQHFAAELS
GRYKKERKALAGIALTTDTSALSAIGNDYGFEFVFSRQVEALGNEGDVLIGISTSGKSPNVLEALKKA
RELGMLCIGLSGKGGGKMNDLCDHCLVVPSDDTARIQEMHILIIHTLCQIIDEAF(LEHHHHHH)
(SEQ ID NO: 57)
SLITLVELEWLEHQLIVQLSERLKGQIAKVGELLCRALKNGGKILICGNGGSAADAQHFAAELSGRYK
KERKALAGIALTTDTSALSAIGNDYGFEFVFSRQVEALGNEGDVLIGISTSGKSPNVLEALKKARELG
MLCIGLSGKGGGKMNDLCDHCLVVPSDDTARIQEMHILIIHTLCQIIDEAF
B4
(SEQ ID NO: 58)
(MG)SLITLVELEWLEHQLIVQLSERLKGQIAKVGELLCRALKNGGKILICGNGGSAADAQHFAAELS
GRYKKERKALAGIALTTDTSALSAIGNDYGFEYVFARQVEALGNEGDVLIGISTSGKSPNVLEALKKA
RELGMLCIGLSGKGGGKMNDLCDMCLVVPSDDTARIQEMHILIIHTLCQIIDEAF(ELHHHHHH)
(SEQ ID NO: 59)
SLITLVELEWLEHQLIVQLSERLKGQIAKVGELLCRALKNGGKILICGNGGSAADAQHFAAELSGRYK
KERKALAGIALTTDTSALSAIGNDYGFEYVFARQVEALGNEGDVLIGISTSGKSPNVLEALKKARELG
MLCIGLSGKGGGKMNDLCDHCLVVPSDDTARIQEMHILIIHTLCQIIDEAF
Di_13_0B
(SEQ ID NO: 60)
MSLITLVELEWLEHQLIVQLSERLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_1B
(SEQ ID NO: 61)
SSLIALVHLEWLEAQLATVLSSSLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILITHTLCQIIDEAF
Di_13_2B
(SEQ ID NO: 62)
SSLIALVYLEWLEAQLATVLSSSLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_3B
(SEQ ID NO: 63)
SSLIALVELEWLEAQLATVNSSDLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSCKGGGKMNDLCDHCLVVPSDDTARIQEMHILIIHTLCQIIDEAF
Di_13_4B
(SEQ ID NO: 64)
SSLIALVYLEWLEAQLATVNSSDLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_5B
(SEQ ID NO: 65)
SSLIALVYLEWLEAQLATVNSSDLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_6B
(SEQ ID NO: 66)
SSLIALVHLEWLEAQLATVNSSDLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_7B
(SEQ ID NO: 67)
SSLIALVYLEWLEAQLATQLSSRLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_8B
(SEQ ID NO: 68)
SSLIALVHLEWLEAQLATQLSSRLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_9B
(SEQ ID NO: 69)
SSLIALVYLEWLEAQLATQLSSRLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_10B
(SEQ ID NO: 70)
SSLITLVEREWIRHQLAVQNSSDLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_11B
(SEQ ID NO: 71)
SSLITLVEREWIRHQLAVQLSSRLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_12B
SSLIALVELEWLRHQLAVVLSSSLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_13B
(SEQ ID NO: 73)
SSLIALVELEWLRHQLAVVDSSDLKGQIAKVGELLCRALKNGGKILICGNGGSAADAQHFAAELSGRY
KKERKALAGIALTTDTSALSAIGNDYGFEEVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_14B
(SEQ ID NO: 74)
SSLIALVELEWLRHQLAVVNSSDLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_15B
(SEQ ID NO: 75)
ASLIQLVELEWIKHQLIVQASARLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_16B
(SEQ ID NO: 76)
ASLIQLVHLEWIKHQLIVQASARLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_17B
(SEQ ID NO: 77)
SSLITLVELEWLAHQLAVELSSSLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_18B
(SEQ ID NO: 78)
SSLITLVEEEWLEHQLAVELSSSLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_19B
(SEQ ID NO: 79)
SSLITLVEEEWLAHQLAVEDSSSLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_20B
(SEQ ID NO: 80)
SSLITLVEEEWLAHQLAVENSSSLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSCKGGGKMNDLCDHCLVVFSDDTARIQEMHILIIHTLCQIIDEAF
Di_13_21B
(SEQ ID NO: 81)
KSLIQLVEKEWLKHQLAVVRSERLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF
Di_13_22B
(SEQ ID NO: 82)
ASLIQLVELEWIKHQLAVQRSARLKGQIAKVGELLCRALKNGGKILICGNGGSAADAQHFAAELSGRY
KKERKALAGIALTTDTSALSAIGNDYGFEFVFSRQVEALGNEGDVLIGISTSGKSPNVLEALKKAREL
GMLCIGLSGKGGGKMNDLCDHCLVVPSDDTARIQEMHILIIHTLCQIIDEAF

In one embodiment, underlined residues of the polypeptide are conserved relative to the reference amino acid sequence. In these embodiments, the underlined residues comprise the region of the polypeptide involved in forming a rigid interface of 2D protein materials when homo-oligomers of these “second” polypeptides co-assemble with homo-oligomers of the “first” polypeptides, embodiments of which are described above.

The polypeptides of this second aspect may comprise one or more additional functional peptide domains. Any functional domain may be added as deemed appropriate for an intended use. Exemplary embodiments of such fusion proteins include, but are not limited to, polypeptides having at least 50%, 55%, 60%, 65%, 70%, 75%, 80%, 85%, 90%, 91%, 92%, 93%, 94%, 95%, 96%, 97%, 98%, or 99% sequence identity to the amino acid sequence selected from the group consisting of SEQ ID NO:83-99 and 106, wherein residues in parentheses are optional and may be present or absent.

Di_13_B_2_C_RFP
B comp. C-term fusion
(SEQ ID NO: 83)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGISGKGGGKMNDLCDHNLVVPSDDTARIQEMHILIIHTLCQIIDEAFGSGSSKGEELFTG
VVPILVELDGDVNGHKFSVSGEGEGDATYGKLTLKFICTTGKLPVPWPTLVTTLSWGVQCFARYPDHM
KQHDFFKSAMPEGYVQERTIFFKDDGNYKTRAEVKFEGDTLVNRIELKGIDFKEDGNILGAKLEYNYF
SDNVYITADKQKNGIKANFKIRHNIEDGGVQLADGYQQNTPIGDGPVLLPDNHYLSTQSKLSKDPNEK
RDHMVLLEFVTAAGITLGMDELYLE(HHHHHH)
Di_13_B_2_C_spyTag
B comp. C-term fusion for peptide fusion
(SEQ ID NO: 84)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGSGSGAHIVMVDA
YKPTKGLE(HHHHHH)
Di13B2_C_mCherry
B comp. C-term fusion
(SEQ ID NO: 85)
(M)GSLITIVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGSGSGSGGSVSKG
EEDNMAIIKEFMRFKVHMEGSVNGHEFEIEGEGEGRPYEGTQTAKLKVTKGGPLPFAWDILSPQFMYG
SKAYVKHPADIPDYLKLSFPEGFKWERVMNFEDGGVVTVTQDSSLQDGEFIYKVKLRGTNFPSDGPVM
QKKTMGWEASSERMYPEDGALKGEIKQRLKLKDGGHYDAEVKTTYKAKKPVQLPGAYNVNIKLDITSH
NEDYTIVEQYERAEGRHSTGGMDELYKLE(HHHHHH)
Di13B2_C_mCherryEm
B comp. C-term fusion
(SEQ ID NO: 86)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGSGSGSGGSVSKG
EAVIKEFMRFKVHMEGSMNGHEFEIEGEGEGRPYEGTQTAKLKVTKGGPLPFSWDILSPQFMYGSRAF
TKHPADIPDYYKQSFPEGFKWERVMNFEDGGAVTVTQDTSLEDGTLIYKVKLRGTNFPPDGPVMQKKT
MGWEASTERLYPEDGVLKGDIKMALRLKDGGRYLADFKTTYKAKKPVQMPGAYNVDRKLDITSHNEDY
TVVEQYERSEGRHSTGGMDELYKLE(HHHHHH)
Di13B2_C_mKate2
B comp. C-term fusion
(SEQ ID NO: 87)
(M)GSLITLVELEWLEHQLIVQLSERLKGQIAKVGELLCECLKNGGKILICGNGGSAADAQHFAAELS
GRYKKERKALAGIALTTDISALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGSGSGSGGSMVSE
LIKENMHMKLYMEGTVNNHHFKCTSEGEGKPYEGTQTMRIKAVEGGPLPFAFDILATSFMYGSKTFIN
HTQGIFDFFKQSFPEGFTWERVTTYEDGGVLTATQDTSLQDGCLIYNVKIRGVNFPSNGPVMQKKTLG
WEASTETLYPADGGLEGRADMALKLVGGGHLICNLKTTYRSKKPAKNLKMPGVYYVDRRLERIKEADK
ETYVEQHEVAVARYCDLPSKLGHRLE(HHHHHH)
Di13B2_C_mKO2
B comp. C-term fusion
(SEQ ID NO: 88)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGSGSGSGGSVSVI
KPEMKMRYYMDGSVNGHEFTIEGEGTGRPYEGHQEMTLRVTMAEGGPMPFAFDLVSHVFCYGHRVFTK
YPEEIPDYFKQAFPEGLSWERSLEFEDGGSASVSAHISLRGNTFYHKSKFTGVNFPADGPIMQNQSVD
WEPSTEKITASDGVLRGDVTMYLKLEGGGNHKCQMKTTYKAAKEILEMPGDHYIGHRLVRKTEGNITE
QVEDAVAHSLE(HHHHHH)
Di13B2_C_mTagBFF2
B comp. C-term fusion
(SEQ ID NO: 89)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGISGKGGGKMNDLCDHNLVVPSDDTARIQEMHILIIHTLCQIIDEAFGSGSGSGGSMVSK
GEELIKENMHMKLYMEGTVDNHHFKCTSEGEGKPYEGTQTMRIKVVEGGPLPFAFDILATSFLYGSKT
FINHTQGIPDFFKQSFPEGFTWERVTTYEDGGVLTATQDTSLQDGCLIYNVKIRGVNFTSNGPVMQKK
TLGWEAFTETLYPADGGLEGRNDMALKLVGGSHLIANAKTTYRSKKPAKNLKMPGVYYVDYRLEPIKE
ANNETYVEQHEVAVARYCDLPSKLGHKLNLE(HHHHHH)
Di13B2_C_mTurq2_sf
B comp. C-term fusion
(SEQ ID NO: 90)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGSGSGSGGSSKGE
ELFTGVVPILVELDGDVNGHKFSVRGEGEGDATNGKLTLKFICTTGKLPVPWPTLVTTLSWGVQCFAR
YPDHMKQHDFFKSAMPEGYVQERTISFKDDGTYKTRAEVKFEGDTLVNRIELKGIDFKEDGNILGHKL
EYNYESDNVYITADKQKNGIKANFKIRHNVEDGSVQLADHYQQNTPIGDGPVLLPUNHILSTQSVLSK
DPNEKRDHMVLLEFVTAAGITHGMDELYLE(HHHHHH)
Di13B2_C_RFP
B comp. C-term fusion
(SEQ ID NO: 91)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGSGSGSGGSELIK
ENMHMKLYMEGTVNNHHFKATSEGEGKPYEGTQTMRIKVVEGGPLPFAFDILATSFMYGSRTFIKHTQ
GIPDFFKQSFPEGFTWERVTTYEDGGVLTATQDTSLQDGMLIYNVKIRGVNFPSNGPVMQKKTLGWEA
NTEMLYPADGGLEGRSDMALKLVGGGHLIVNFKTTYRSKKPAKNLKMPGVYYVDHRLERIKEADKETY
VEQHEVAVARYLE(HHHHHH)
Di1382loop2B4_C_mCherryEm
cyclic B comp, with C-term fusion
(SEQ ID NO: 92)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGGGKDRNGGSLIT
LVELEWLEHQLIVQLSERLKGQIAKVGELLCRALKNGGKILICGNGGSAADAQHFAAELSGRYKKERK
ALAGIALTTDTSALSAIGNDYGFEYVFARQVEALGNEGDVLIGISTSGKSPNVLEALKKARELGMLCI
GLSGKGGGKMNDLCDHCLVVPSDDTARIQEMHILIIHTLCQIIDEAFGSGSGSGGSVSKGEAVIKEFM
RFKVHMEGSMNGHEFEIEGEGEGRPYEGTQTAKLKVTKGGPLPFSWDILSPQFMYGSRAFTKHPADIP
DYYKQSFPEGFKWERVMNFEDGGAVTVTQDTSLEDGTLIYKVKLRGTNFFPDGPVMQKKTMGWEASTE
RLYPEDGVLKGDIKMALRLKDGGRYLADFKTTYKAKKPVQMPGAYNVDRKLDITSHNEDYTVVEQYER
SEGRHSTGGMDELYKLE(HHHHHH)
Di13B2loop2B4_C_MKate2
cyclic 8 comp. with C-term fusion
(SEQ ID NO: 93)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGGGKDRNGGSLIT
LVELEWLEHQLIVQLSERLKGQIAKVGELLCRALKNGGKILICGNGGSAADAQHFAAELSGRYKKERK
ALAGIALTTDTSALSAIGNDYGFEYVFARQVEALGNEGDVLIGISTSGKSPNVLEALKKARELGMLCI
GLSGKGGGKMNDLCDHCLVVPSDDTARIQEMHILIIHTLCQIIDEAFGSGSGSGGSMVSELIKENMHM
KLYMEGTVNNHHFKCTSEGEGKPYEGTQTMRIKAVEGGPLPFAFDILATSFMYGSKTFINHTQGIPDF
FKQSFPEGFTWERVTTYEDGGVLTATQDTSLQDGCLIYNVKIRGVNFPSNGPVMQKKTLGWEASTETL
YPADGGLEGRADMALKLVGGGHLICNLKTTYRSKKPAKNLKMPGVYYVDRRLERIKEADKETYVEQHE
VAVARYCDLPSKLGHRLE(HHHHHH)
Di13B2loop2B4_C_mkO2
cyclic B comp. with C-term fusion
(SBQ ID NO: 94)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGGGKDRNGGSLIT
LVELEWLEHQLIVQLSERLKGQIAKVGELLCRALKNGGKILICGNGGSAADAQHFAAELSGRYKKERK
ALAGIALTTDTSALSAIGNDYGFEYVFARQVEALGNEGDVLIGISTSGKSPNVLEALKKARELGMLCI
GLSGKGGGKMNDLCDHCLVVPSDDTARIQEMHILIIHTLCQIIDEAFGSGSGSGGSVSVIKPEMKMRY
FKQAFPEGLSWERSLEFEDGGSASVSAHISLRGNTFYHKSKFTGVNFPADGPIMQNQSVDWEPSTEKI
TASDGVLKGDVTMYLKLEGGGNHKCQMKTTYKAAKEILEMPGDHYIGHRLVRKTEGNITEQVEDAVAH
SLE(HHHHHH)
Di13B2loop2B4_C_mTagBFP2
cyclic B comp. with C-term fusion
(SEQ ID NO: 95)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGGGKDRNGGSLIT
LVELEWLEHQLIVQLSERLKGQIAKVGELLCRALKNGGKILICGNGGSAADAQHFAAELSGRYKKERK
ALAGIALTTDTSALSAIGNDYGFEYVFARQVEALGNEGDVLIGISTSGKSPNVLEALKKARELGMLCI
GLSGKGGGKMNDLCDHCLVVPSDDTARIQEMHILIIHTLCQIIDEAFGSGSGSGGSMVSKGEELIKEN
MHMKLYMEGTVDNHHFKCTSEGEGKPYEGTQTMRIKVVEGGPLPFAFDILATSFLYGSKTFINHTQGI
PDFFKQSFPEGFTWERVTTYEDGGVLTATQDTSLQDGCLIYNVKIRGVNFTSNGPVMQKKTLGWEAFT
ETLYPADGGLEGRNDMALKLVGGSHLIANAKTTYRSKKPAKNLKMPGVYYVDYRLERIKEANNETYVE
QHEVAVARYCDLPSKLGHKLNLE(HHHHHH)
Di13B2loop2B4_C_mTurq2_sf
cyclic B comp. with C-term fusion
(SEQ ID NO: 96)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGCKMNDLCDHNLVVPSDDTARIQEMHILIIHTLCQIIDEAFGGGKDRNGGSLIT
LVELEWLEHQLIVQLSERLKGQIAKVGELLCRALKNGSKILICGNSGSAADAQHFAAELSGRYKKERK
ALAGIALTTDTSALSAIGNDYGFEYVFARQVEALGNEGDVLIGISTSGKSPNVLEALKKARELGMLCI
GLSGKGGGKMNDLCDHCLVVPSDDTARIQEMHILTINTLCQTIDEAFGSGSGSGGSSKGEELFTGVVP
ILVELDGDVNGHKFSVRGEGEGDATNGKLTLKFICTTGKLPVPWPTLVTTLSWGVQCFARYPDHMKQH
DFFKSAMPEGYVQERTISFKDDGTYKTRAEVKFEGDTLVNPIELKGIDFKEDGNILGBKLEYNYFSDN
VYITADKQKNGIKANFKIRHNVEDGSVQLADHYQQNTPIGDGFVLLPDNHYLSTQSVLSKDPNEKRDH
MVLLEFVTAAGITHGMDELYLE(HHHHHH)
Di13B2loop2B4_C_RFP
cyclic B comp. with C-term fusion
(SEQ ID NO: 97)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGGGKDRNGGSLIT
LVELEWLEHQLIVQLSERLKGQIAKVGELLCRALKNGSKILICGNSGSAADAQHFAAELSGRYKKERK
ALAGIALITDTSALSAIGNDYGFEYVFARQVEALGNEGDVLIGISTSGKSENVLEALKKARELGMLCI
GLSGKGGGKMNDLCDHCLVVPSDDTARIQEMHILIIHTLCQIIDEAFGSGSGSGGSELIKENMHMKLY
MEGTVNNHHFKATSEGEGKEYEGTQTMRIKVVEGGPLPFAFDILATSFMYGSRTFIKHTQGIPDFFKQ
SFPEGFTWERVTTYEDGGVLTATQDTSLQDGMLIYNVKIRGVNFPSNGPVMQKKTLGWEANTEMLYPA
DGGLEGRSDMALKLVGGGHLIVNFKTTYRSKKPAKNLKMPGVYYVDHRLERIKEADKETYVEQHEVAV
ARYLE(HHHHHH)
Di13B2loop2B4_C_SYFP2_sf
cyclic B comp. with C-term fusion
(SEQ ID NO: 98)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGGGKDRNGGSLIT
LVELEWLENQLIVQLSERLKGQIAKVGELLCRALKNGGKILICGNGGSAADAQHFAAELSGRYKKERK
ALAGIALTTDTSALSAIGNDYGFEYVFARQVEALGNEGDVLIGISTSGKSPNVLEALKKARELGMLCI
GLSGKGGGKMNDLCDHCLVVPSDDTARIQEMHILIIHTLCQIIDEAFGSGSGSGGSSKGEELFTGVVP
ILVELDGDVNGEKFSVRGEGEGDATNGKLTLKLICTTGKLPVPWPTLVTTLGYGVQCFARYPDHMKQH
DFFKSAMPEGYVQERTISFRDDGTYKTRAEVKFEGDTLVNRIELKGIDFKEDGNILGHKLEYNYNSHN
VYITADKQKNGIKANFKIRHNVEDGSVQLADHYQQNTPIGDGPVLLPDNHYLSYQSVLSKDPNEKRDH
MVLLEFVTAAGITHGMDELYLE(HHHHHH)
B_c_mScarlet
cyclic B comp. with C-term fusion
(SEQ ID NO: 99)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGGGKDANGGSLIT
LVELEWLSHQLIVQLSERLKGQIAKVGELLCRALKNGGKILICGNGGSAADAQHFAAELSGRYKKERK
ALAGIALTTDTSALSAIGNDYGFEYVFARQVEALGNEGDVLIGISTSGKSPNVLEALKKARELGMLCI
GLSGKGGGKMNDLCDHCLVVPSDDTARIQEMHILIIHTLCQIIDEAFGSGGSGTSGSAMVSKGEAVIK
EFMRFKVHMEGSMNGHEFEIEGEGEGRPYEGTQTAKLKVTKGGPLPFSWDILSPQFMYGSRAFTKHPA
DIPDYYKQSFPEGFKWERVMNFEDGGAVTVTQDTSLEDGTLIYKVKLRGTNFPPDGPVMQKKTMGWEA
STERLYPEDGVLKGDIKMALRLKDGGRYLADFRTTYKAKKPVQMPGAYNVDEKLDITSHNEDYTVVEQ
YERSEGRHSTGGMDELYKLE(HHHHHH)
Di13B2L4B4 cyclic B compo. With His tagged C-term
(SEQ ID NO: 101)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGGSGGSGGSLITL
VELEWLEHQLIVQLSERLKGQIAKVGELLCRALKNGGKILICGNGGSAADAQHFAAELSGRYKKERKA
LAGIALTTDTSALSAIGNDYGFEYVFARQVEALGNEGDVLIGISTSGKSPNVLEALKKARELGMLCIG
LSGKGGGKMNDLCDHCLVVPSDDTARIQEMHILIIHTLCQIIDEAFLE(HHHHHH)

### B(c) Fused to a ECFP-I146N

>Di13BL4B_cECFP_I146N
(SEQ ID NO: 102)
(M)GSLITLVELEWLEHQLIVQLSERLKGQIAKVGELLCECLKNGGKILI
CGNGGSAADAQHFAAELSGRYKKERKALAGIALTTDTSALSAIGNDYGFE
FVFSRQVEALGNEGDVLIGISTSGKSPNVLEALKKARELNMLCIGLSGKG
GGKMNDLCDHNLVVPSDDTARIQEMHILIIHTLCQIIDEAFGGETSSKQD
LITLVELEWLEHQLIVQLSERLKGQIAKVGELLCRALKNGGKILICGNGG
SAADAQHFAAELSGRYKKERKALAGIALTTDTSALSAIGNDYGFEYVFAR
QVEALGNEGDVLIGISTSGKSPNVLEALKKARELGMLCIGLSGKGGGKMN
DLCDHCLVVPSDDTARIQEMHILIIHTLCQIIDEAFGSGSGSGSGMVSKG
EELFTGVVPILVELDGDVNGHKFSVSGEGEGDATYGKLTLKFICTTGKLP
VPWPTLVTTLTWGVQCFSRYPDHMKQHDFFKSAMPEGYVQERTIFFKDDG
NYKTRAEVKFEGDTLVNRIELKGIDFKEDGNILGHKLEYNYNSHNVYITA
DKQKNGIKANFKIRHNIEDGSVQLADHYQQNTPIGDGPVLLPDNHYLSTQ
SALSKDPNEKRDHMVLLEFVTAAGITLGMDELYKLE(HHHHHH)

B(c) Fused to mCherry

>Di13B2loop2B4_C_mCherryEm
(SEQ ID NO: 103)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGGGKDRNGGSLIT
LVELEWLEHQLIVQLSERLKGQIAKVGELLCRALKNGGKILICGNGGSAADAQHFAAELSGRYKKERK
ALAGIALTTDTSALSAIGNDYGFEYVFARQVEALGNEGDVLIGISTSGKSPNVLEALKKAPELGMLCI
GLSGKGGGKMNDLCDHCLVVPSDDTARIQEMHILIIHTLCQIIDEAFGSGSGSGGSVSKGEAVIKEFM
RFKVHMEGSMNGHEFEIEGEGEGRPYEGTQTAKLKVTKGGPLPFSWDILSPQFMYGSRAFTKHPADIP
DYYKQSFPEGFKWERVMNFEDGGAVTVTQDTSLEDGTLIYKVKLRGTNFPPDGPVMQKKTMGWEASTE
RLYPEDGVLKGDIKMALRLKDGGRYLADFKTTYKAKKPVQMPGAYNVDRKLDITSHNEDYTVVEQYER
SEGRHSTGGMDELYKLE(HHHHHH)
Di13B2L4B4_SC
(SEQ ID NO: 104)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIIHTLCQIIDEAFGGETSSKQDLITL
VELEWLEHQLIVQLSERLKGQIAKVGELLCRALKNGGKILICGNGGSAADAQHFAAELSGRYKKERKA
LAGIALTTDTSALSAIGNDYGFEYVFARQVEALGNEGDVLIGISTSGKSPNVLEALKKARELGMLCIG
LSGKGGGKMNDLCDHCLVVPSDDTARIQEMHILIIHTLCQIIDEAFGSGSGSGSGAMVDTLSGLSSEQ
GQSGDMTIEEDSATHIKFSKRDEDGKELAGATMELRDSSGKTISTWISDGQVKDFYLYPGKYTFVETA
APDGYEVATAITFTVNEQGQVTVNGKATKGDAHIGLE(HHHHHH)
Di13BLB_C_GFP
(SEQ ID NO: 105)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMLCIGLSGKGGGKMNDLCDHNLVVPSDDTARIQEMHILIINTLCQIIDEAFGGGKDRNGGSLIT
LVELEWLEHQLIVQLSERLKGQIAKVGELLCRALKNGGKILICGNGGSAADAQHFAAELSGRYKKERK
ALAGIALTTDTSALSAIGNDYGFEYVFARQVEALGNEGDVLIGISTSGKSPNVLEALKKARELGMLCI
GLSGKGGGKMNDLCDHCLVVPSDDTARIQEMHILIIHTLCQIIDEAFGSGSGSGGSSKGEELFTGVVP
ILVELDGDVNGHKFSVRGEGEGDATNGKLTLKFICTTGKLPVPWPTLVTTLTYGVQCFARYPDHMKQH
DFFKSAMPEGYVQERTISFKDDGTYKTRAEVKFEGDTLVNRIELRGIDFKEDGNILGHKLEYNFNSHN
VYITADKQKNGIKANFKIRHNVEDGSVQLADHYQQNTPIGDGPVLLPDNHYLSTQSVLSKDPNEKRDH
MVLLEFVTAAGITHGLYLE(HHHHHH)
Di13B2loop2B4_C_mCherry
(SEQ ID NO: 106)
(M)GSLITLVELEWLEHQLIVQLSERLKGQIAKVGELLCECLKNGGKILICGNGGSAADAQHFAAELS
GRYKKERKALAGIALTTDTSALSAIGNDYGFEFVFSRQVEALGNEGDVLIGISTSGKSPNVLEALKKA
RELNMICIGLSGKGGGKMNDLCDHNLVVPSDDTARIQEMHILIIHTLCQIIDEAFGGGKDRNGGSLIT
LVELEWLEHQLIVQLSERLKGQIAKVGELLCRALKNGGKILICGNGGSAADAQHFAAELSGRYKKERK
ALAGIALTTDTSALSAIGNDYGFEYVFARQVEALGNEGDVLIGISTSGKSPNVLEALKKARELGMLCI
GLSGKGGGKMNDLCDHCLVVPSDDTARIQEMHILITHTLCQIIDEAFGSGSGSGGSVSKGEEDNMAII
KEFMRFKVHMEGSVNGHEFEIEGEGEGRPYEGTQTAKLKVTKGGPLPFAWDILSPQFMYGSKAYVKHP
ADIPDYLKLSFPEGFKWERVMNFEDGGVVTVTQDSSLQDGEFIYKVKLRGTNFPSDGPVMQKKTMGWE
ASSERMYPEDGALKGEIKQRLKIKDGGHYDAEVKTTYKAKKPVQLPGAYNVNIKLDITSHNEDYTIVE
QVERAEGRHSTGGMDELYKLE(HHHHHH)

In one embodiment the optional residues are present in the polypeptides and considered in determining percent identity relative to the reference sequence; in other embodiments, the optional residues are not present and are not considered in determining percent identity relative to the reference sequence.

As discussed in detail herein, the polypeptides of this second aspect self-assemble into homo-oligomers comprising a second interface region that can interact with a first interface region of homo-oligomers of the polypeptides of the first aspect of the disclosure. Thus, in another embodiment, the disclosure provides homo-oligomers of the polypeptide of any embodiment or combination of embodiments of the second aspect of the disclosure.

In one embodiment, the homo-oligomer is a cyclic homo-oligomer. In a further embodiment, the cyclic homo-oligomer comprises a homo-oligomer of a polypeptide comprising an amino acid sequence having at least 50%, 55%, 60%, 65%, 70%, 75% 80%, 85%, 90%, 91%, 92%, 93%, 94%, 95%, 96%, 97%, 98%, or 99% sequence identity to the amino acid sequence of SEQ ID NO:101 (Di13B2L4B4 cyclic B compo.), wherein residues in parentheses are optional. In one embodiment the optional residues are present in the polypeptides and considered in determining percent identity relative to the reference sequence; in other embodiments, the optional residues are not present and are not considered in determining percent identity relative to the reference sequence.

Di13B2B4B4 cyclic B compo, With His tagged C-term
(SEQ ID NO: 101)
(M)GSLITLVELEWLEHQLIVQLSERLKGQIAKVGELLCECLANGGKILI
CGNGGSAADAQHFAAELSGRYKKERKALAGIALTTDTSALSAIGNDYGFE
FVFSRQVEALGNEGDVLIGTSTSGKSPNVLEALKKARELNMLCIGLSGKG
GGKMNDLCDHNLVVPSDDTARIQEMHILIIHTLCQIIDEAFGGSGGSGGS
LITLVELEWLEHQLIVQLSERLKGQIAKVGELLCRALKNGGKILICGNGG
SAADAQHFAAELSGRYKKERKALAGIALTTDTSALSAIGNDYGFEYVFAR
QVEALGNEGDVLIGISTSGKSPNVLEALKKARELGMLCIGLSGKGGGKMN
DLCDHCLVVPSDDTARIQEMHILIIHTLCQIIDEAFLE(HHHHHH)

In a third aspect, the present disclosure provides nucleic acids, including isolated nucleic acids, encoding the polypeptides of any embodiment or combination of embodiments of the present disclosure that can be genetically encoded. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, secretory signals, nuclear localization signals, and plasma membrane localization signals. It will be apparent to those of skill in the art, based on the teachings herein, what nucleic acid sequences will encode the polypeptides of the invention.

In a fourth aspect, the present disclosure provides expression vectors comprising the nucleic acid of any aspect of the invention operatively linked to a suitable control sequence. “Expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector. In some embodiments, the expression vector can be administered in the methods of the disclosure to express the polypeptides in vivo for therapeutic benefit. In non-limiting embodiments, the expression vectors can be used to transfer or transduce cell therapeutic targets (including but not limited to CAR-T cells or tumor cells) to effect the therapeutic methods disclosed herein.

In a fifth aspect, the present disclosure provides host cells that comprise the expression vectors and/or nucleic acids disclosed herein, wherein the host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press), Culture of Animal Cells: A Manual of Basic Technique, 2^{nd} Ed. (R. I. Freshney, 1987. Liss, Inc. New York, NY)). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium.

In a sixth aspect, the disclosure provides two-dimensional protein structures, comprising a first polypeptide and a second polypeptide, wherein
(a) the first polypeptide and the second polypeptide are different;
(b) the first polypeptide self-assembles into a first homo-oligomer, wherein the first homo-oligomer comprises a first interface region, said first interface region having a rotational symmetry;
(c) the second polypeptide self-assembles into a second homo-oligomer, wherein the second homo-oligomer comprises a second interface region, said second interface region having a rotational symmetry, and
(d) the first homo-oligomer and the second homo-oligomer interact via the first interface region and the second interface region to form a rigid interface.

As described herein, the inventors disclose a computational method to generate de-novo binary 2D non-covalent co-assemblies by designing rigid asymmetric interfaces between two distinct protein dihedral building-blocks. The designed array components are soluble at mM concentrations, but when combined at nM concentrations, rapidly assemble into nearly-crystalline micrometer-scale p6m arrays nearly identical to the computational design model in vitro and in cells without the need of a two-dimensional support. Because the material is designed from the ground up, the components can be readily functionalized, and their symmetry reconfigured, enabling formation of ligand arrays with distinguishable surfaces to drive extensive receptor clustering, downstream protein recruitment, and signaling. The 2D protein materials can impose order onto fundamentally disordered substrates like cell membranes. In sharp contrast to previously characterized cell surface receptor binding assemblies such as antibodies and nanocages, which are rapidly endocytosed, large arrays of the present 2D protein materials assembled at the cell surface suppress endocytosis in a tunable manner, providing potential therapeutic benefits for extending receptor engagement and immune evasion.

Specific exemplary embodiments of the polypeptides and homo-oligomers are provided herein in the first and second aspects of the disclosure. The examples provide detailed rules for generating other such 2D protein arrays starting from a variety of different initial polypeptides.

The first and second homo-oligomers do not independently interact to form larger structures and are stable in solution. Co-assembly into a two dimensional protein structure only occurs when the first homo-oligomer and the second homo-oligomer interact via the rigid interface. As used herein, “rigid” means that the peptide region that takes part in the interface is a structurally well-defined secondary structure (i.e., known down to a certain defined x-Angstrom resolution). This is very different than interfaces based on peptide fusions where a flexible linker connects the building block component and the peptide and so its position is not well defined and only estimated.

The homo-oligomers in this embodiment have “pseudo-dihedral symmetry” in that the homo-oligomer array forming interface regions have a dihedral symmetry, but the entire homo-oligomer is not required to be dihedral.

In one embodiment of this sixth aspect, the first interface region and the second interface regions comprise alpha-helical domains. In this embodiment, each monomer (i.e.: first polypeptide and second polypeptide) may provide a single alpha helix to the rigid interface between the two homo-oligomers, but each first homo-oligomer provides two alpha helices and each second homo-oligomer provides two alpha helices to the rigid interface.

In a seventh aspect, the disclosure also provides two-dimensional protein structures, comprising a first polypeptide and a second polypeptide, wherein
(a) the first polypeptide and the second polypeptide are different;
(b) the first polypeptide self-assembles into a first homo-oligomer;
(c) the second polypeptide self-assembles into a second homo-oligomer;
(d) the first homo-oligomer and the second homo-oligomer interact to form a rigid interface; and wherein
(e) one or both of the first homo-oligomer and the second homo-oligomer has a cyclic pseudo-dihedral symmetry.

This aspect is particularly preferred to form arrays on soft substrates, including but not limited to cells. As used herein, “cyclic pseudo-dihedral symmetry” means a cyclic homo-oligomer in which a subset of the polypeptide residues display a dihedral point symmetry. The polypeptides may be any that can be part of a pair of distinct or identical proteins, which independently form dihedral or pseudo-dihedral homo-oligomers, and contact each other, while one of their in-plane symmetry/pseudo symmetry axis coincide and each interact with 3 residues or more which belong to rigid secondary structure, either a helix or a beta sheet.

In one embodiment of this seventh aspect, the interface comprises an interface between an alpha-helical domain of the first polypeptide and an alpha-helical domain of the second polypeptide. In one cyclic pseudo-dihedral embodiment, each monomer (i.e.: first polypeptide and second polypeptide) may provide two alpha helices connected by either a loop domain (case of component B) or by numerous secondary structures (case of the A component) to the rigid interface.

In one embodiment of the sixth or seventh aspect of the disclosure, each of the first polypeptide and the second polypeptide may comprise a plurality (2, 3, 4, 5, 6, 7, or more) alpha helical domains separate by loop domains.

In another embodiment of the sixth or seventh aspect of the disclosure, the interface comprises (a) a region of the first polypeptide within 25 amino acids from the first polypeptide C-terminus, and (b) a region of the second polypeptide within 25 amino acids from the second polypeptide N-terminus.

In another embodiment of the sixth or seventh aspect of the disclosure
(a) the first polypeptide comprises a secondary structure as shown below, wherein positions in parentheses are optional and may be present or absent:

First polypeptide
(LLLLLLLLLLLLLL)LLLLLLHHHLLLHHHHLLLLLLLLLHHHHHHHHH
HHHHHHHLLLHHHHHHHHHHHLLHHHHHHHHHHHHLLLLLLLLLHHHHHH
HHHLHHHHHHHHHHHHHHHHLLLLHHHHHLLLLLLLLLLLLLLLLLLLLL
HHHHHHHHHHHHHHHHLHHHHHHHHHHHHHHHHHHHHHLLLLHHHHHHHH
HHHHHHHHHHHHLHHHHHHHHHHHHHHHHHLL;

(b) the second polypeptide comprises a secondary structure as shown below;

Second polypeptide
LLHHHHHHHHHHHHHHHHHHHHHHHHHHHHHHHHHHHHHHLLLLEEEEEL
HHHHHHHHHHHHHHHLLLLLLLLLLLEEELLLLHHHHHHHHHHLLHHHLL
HHHHHHHLLLLLEEEEEELLLLLHHHHHHHHHHHHLLLEEEEEEELLLHH
HHHHLLEEEEELLLLHHHHHHHHHHHHHHHHHHHHHHL

wherein H represents amino acid residues present in an alpha helix; L represents amino acids present in a loop, and E represents amino acid residues present in a beta sheet, and wherein amino acid insertions may be present in loop regions.

In this embodiment, the polypeptide length is variable, since amino acid insertions may be incorporated into the loop regions. Such insertions may be of any length and amino acid composition as deemed appropriate for an intended purpose. In this embodiment, the first polypeptide is at least 216 amino acids in length and has at least 9 helical domains and loop domains arranged as shown above, and the second polypeptide is at least 183 amino acids in length (i.e.: up to 5 terminal N- and/or C-terminal residues may be removed) and has at least 8 helical domains and at least 9 loop domains with 5 of the loop domains including beta sheet structures as shown above.

In various other embodiments of the sixth or seventh aspects of the disclosure, the first polypeptide comprises a polypeptide of any embodiment or combination of embodiments of the first aspect of the disclosure, and/or the second polypeptide comprises a polypeptide of any embodiment or combination of embodiments of the second aspect of the disclosure.

In a further embodiment, the first polypeptide comprises an amino acid sequence having at least 50%, 55%, 60%, 65%, 70%, 75%, 80%, 85%, 90%, 91%, 92%, 93%, 94%, 95%, 96%, 97%, 98%, or 99% sequence identity to the amino acid sequence of SEQ ID NO:31, wherein residues in parentheses are optional and may be present or absent. In another embodiment, the second polypeptide comprises an amino acid sequence having at least 50%, 55%, 60%, 65%, 70%, 75%, 80%, 85%, 90%, 91%, 92%, 93%, 94%, 95%, 96%, 97%, 98%, or 99% sequence identity to the amino acid sequence of SEQ ID NO:101, wherein residues in parentheses are optional and may be present or absent.

The disclosure also provides two-dimensional protein materials comprising
(a) a first homo-oligomer of a first polypeptide of any embodiment or combination of embodiments of the first aspect of the disclosure; and
(b) a second homo-oligomer of a second polypeptide of any embodiment or combination of embodiments of the second aspect of the disclosure, where the first homo-oligomer and the second homo-oligomer interact at a rigid interface.

In some embodiments, the first homo-oligomer and the second homo-oligomers according to this and other aspects and embodiments disclosed herein may be first and second homo-oligomers according to any embodiment or combination of embodiments disclosed herein. In various embodiments, the first and second homo-oligomers comprise a pair of homo-oligomers comprising an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity to the amino acid sequence selected from the group consisting of the following, wherein optional residues (including any N-terminal methionine residues) may be present or absent:
(a) SEQ ID NOS:2-7 (As1-As3), and SEQ ID NOS:50-59 (13-B4);
(b) Di_13_0A (SEQ ID NO:8) and Di_13_0B (SEQ ID NO:60);
(c) Di_13_1A (SEQ ID NO:9) and Di_13_1B (SEQ ID NO:61);
(d) Di_13_2A (SEQ ID NO:10) and Di_13-2B (SEQ ID NO:62);
(e) Di_13_3A (SEQ ID NO: 11) and Di_13_3B (SEQ ID NO:63);
(f) Di_13_4A (SEQ ID NO:12) and Di_13_4B (SEQ ID NO:64);
(g) Di_13_5A (SEQ ID NO:13) and Di_13_5B (SEQ ID NO:65);
(h) Di_13_6A (SEQ ID NO:14) and Di_13_6B (SEQ ID NO:66);
(i) Di_13_7A (SEQ ID NO:15) and Di_13_7B 9SEQ ID NO:67);
(j) Di_13_8A (SEQ ID NO:16) and Di_13_8B (SEQ ID NO:68);
(k) Di_13_9A (SEQ ID NO:17) and Di_13_9B (SEQ ID NO:69);
(l) Di_13_10A (SEQ ID NO: 18) and Di_13_10B (SEQ ID NO:70);
(m) Di_13_11A (SEQ ID NO: 19) and Di_13_11B (SEQ ID NO:71);
(n) Di_13_12A (SEQ ID NO:20) and Di_13_12B (SEQ ID NO:72);
(o) Di_13_13A (SEQ ID NO:21) and Di_13_13B (SEQ ID NO:73);
(p) Di_13_14A (SEQ ID NO:22) and Di_13_14B (SEQ ID NO:74);
(q) Di_13_15A (SEQ ID NO:23) and Di_13_15B (SEQ ID NO:75);
(r) Di_13_16A (SEQ ID NO:24) and Di_13_16B (SEQ ID NO:76);
(s) Di_13_17A (SEQ ID NO:25) and Di_13_17B (SEQ ID NO:77);
(t) Di_13_18A (SEQ ID NO:26) and Di_13_18B (SEQ ID NO:78);
(u) Di_13_19A (SEQ ID NO:27) and Di_13_19B (SEQ ID NO:79);
(v) Di_13_20A (SEQ ID NO:28) and Di_13_20B (SEQ ID NO:80);
(w) Di_13_21A (SEQ ID NO:29) and Di_13_21B (SEQ ID NO:81);
(x) Di_13_22A (SEQ ID NO:30) and Di_13_22B (SEQ ID NO:82); and
(y) Cyclic A comp. (SEQ ID NO:31) and Cyclic B comp. (SEQ ID NO:101).

In another aspect, the disclosure provides uses of and methods for using the polypeptides, fusion proteins, homo-polymers, 2D protein materials, nucleic acids, recombinant expression vectors, and host cells of any of the preceding claims for any suitable purpose, including but not limited to those described herein. As described herein, the polypeptides, fusion proteins, and homo-polymers may be used, for example, to generate the binary 2D non-covalent co-assemblies that interact at rigid asymmetric interfaces between two distinct protein dihedral building-blocks. The designed array components are soluble at mM concentrations, but when combined at nM concentrations, rapidly assemble into nearly-crystalline micrometer-scale p6m arrays nearly identical to the computational design model in vitro and in cells without the need of a two-dimensional support. The components can be readily functionalized, and their symmetry reconfigured, enabling formation of ligand arrays with distinguishable surfaces to drive extensive receptor clustering, downstream protein recruitment, and signaling. The 2D protein materials can impose order onto fundamentally disordered substrates like cell membranes. In sharp contrast to previously characterized cell surface receptor binding assemblies such as antibodies and nanocages, which are rapidly endocytosed, large arrays of the present 2D protein materials assembled at the cell surface suppress endocytosis in a tunable manner, providing potential therapeutic benefits for extending receptor engagement and immune evasion.

### EXAMPLES

### Abstract

Proteins that assemble into ordered two-dimensional arrays are generally constituted from just one protein component. For modulating assembly dynamics and incorporating more complex functionality, materials composed of two components would have considerable advantages. Here we describe a computational method to generate de-novo binary 2D non-covalent co-assemblies by designing rigid asymmetric interfaces between two distinct protein dihedral building-blocks. The designed array components are soluble at mM concentrations, but when combined at nM concentrations, rapidly assemble into nearly-crystalline micrometer-scale p6m arrays nearly identical to the computational design model in vitro, by TEM and SAXS, and in cells without the need for a two-dimensional support. Because the material is designed from the ground up, the components can be readily functionalized, and their symmetry reconfigured, enabling formation of ligand arrays with distinguishable surfaces to drive extensive receptor clustering, downstream protein recruitment, and signaling. Using AFM on supported bilayers and quantitative microscopy on living cells, we show that arrays assembled on membranes have component stoichiometry and structure similar to arrays formed in vitro, and thus that our material can impose order onto fundamentally disordered substrates like cell membranes. We find further that in sharp contrast to previously characterized cell surface receptor binding assemblies such as antibodies and nanocages, which are rapidly endocytosed, large arrays assembled at the cell surface suppress endocytosis in a tunable manner, with potential therapeutic relevance for extending receptor engagement and immune evasion. Our work paves the way towards synthetic cell biology, where a new generation of multi-protein macroscale materials is designed to modulate cell responses and reshape synthetic and living systems.

### INTRODUCTION

Most previously known ordered protein 2D materials primarily involve single protein components. A de-novo interface design between rigid domains that is stabilized by extensive noncovalent interactions would provide more control over atomic structure and a robust starting point for further structural and functional modulation.

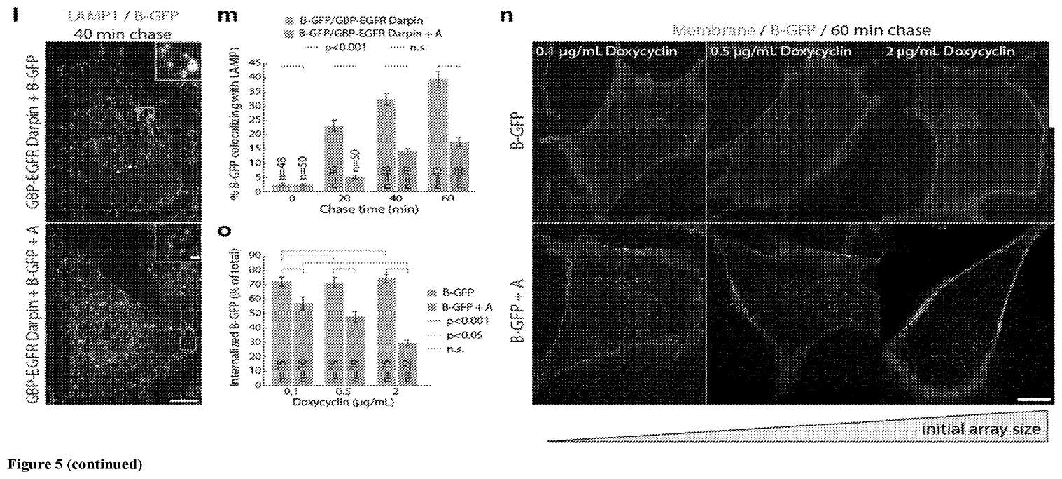

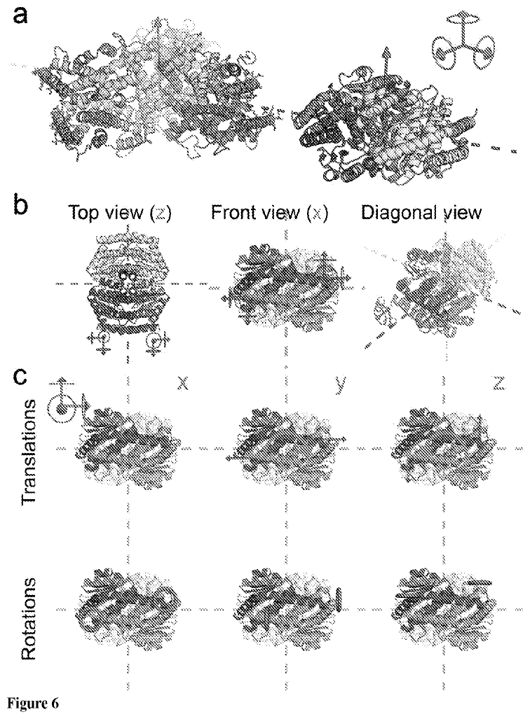

We set out to design two component 2D arrays by engineering de-novo heterotypic (asymmetric) interfaces between dihedral protein homooligomeric building-blocks (BBs). There are 17 distinct plane symmetry groups that define 2D repetitive patterns, but a broader set of unique geometries are available using 3D objects; 33 distinct planar geometries can be generated by combining two objects. The BBs can be either cyclic or dihedral homooligomers oriented in space such that their highest order rotation symmetry (Cₓ: xÎ{2,3,4,6}) is perpendicular to the plane. We chose a subset of the 17 plane symmetry groups (p3 m1, p4m, p6m) that can be generated by introducing a single additional interface between BBs with dihedral symmetry. We chose to use objects with dihedral rather than cyclic symmetry for their additional in-plane 2-fold rotation axes (FIG. 1.a, dashed lines) that intrinsically correct for any deviation from the design model which might otherwise result in out-of-plane curvature (see FIG. 6 for further discussion). This higher symmetry comes at a cost in the number of degrees of freedom (DOF) available for a pair of objects to associate: while cyclic components are constrained in a plane to 4 DOF, for dihedrals the only DOFs are the lattice spacing and discrete rotations of the BBs (the dihedral axes of the two components must be aligned). For example, FIG. 1.a shows a two component 2D lattice generated by placing D3 and D2 BBs on the C3 and C2 axes of the p6m(*632) symmetry group with their in-plane C2 axes coincides. We sampled 2D arrays in the p3m1[D3-D3], p4m[D4-D4, D4-D2], and p6m[D6-D3, D6-D2, D3-D2] groups built from 965 dihedral BBs available in the PDB with D2, D3, D4 and D6 symmetry and x-ray resolution better than 2.5 Å. For each group, all pairs of dihedral BBs were placed with their symmetry axes aligned to those of the group, and the lattice spacing (FIG. 1.a, middle) and the discrete rotations (FIG. 1.a, left) were sampled to identify arrangements with contact regions greater than 400 sq Å and composed primarily of aligned helices. The amino acid sequences at the resulting interfaces between the two building blocks were optimized using Rosettaim combinatorial sequence design to generate low energy interactions across the interface and varying residue chemical characteristics such as to create minimal hydrophobic pockets surrounded by polar residues.

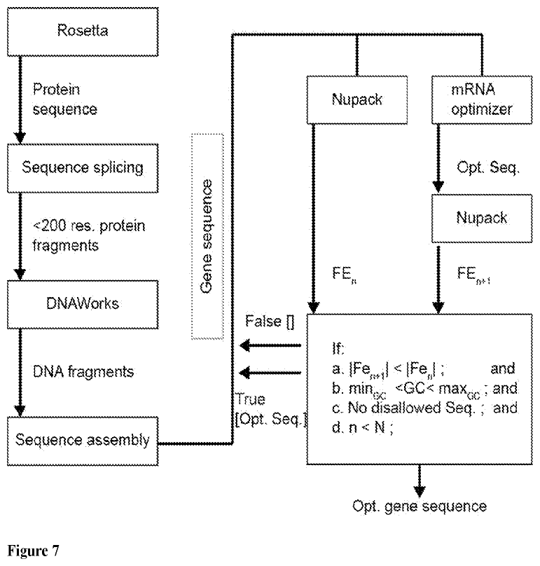

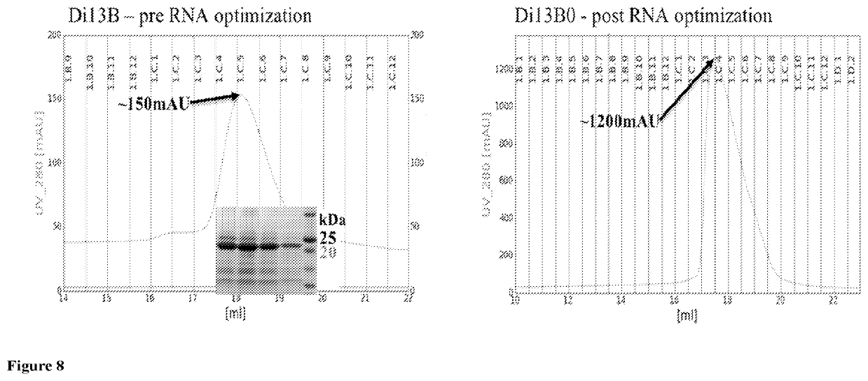

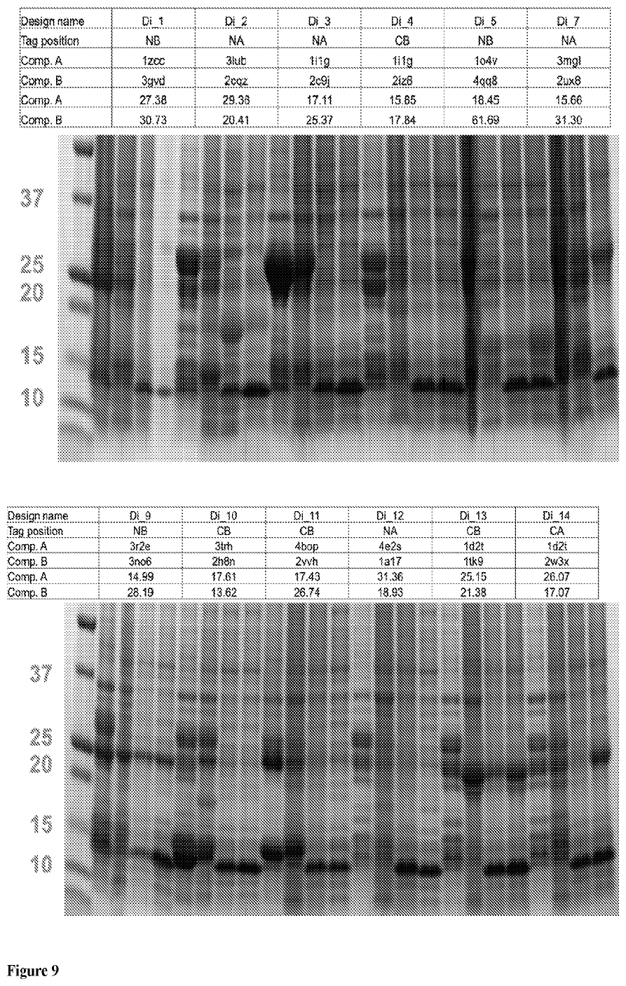

We selected forty-five of the lowest energy designs (2—p3 mL, 10—p4m, and 33—p6m) with high shape complementarity and few buried polar groups not making hydrogen bonds (see FIG. 1.b), encoded them in mRNA optimized genes on a bicistronic plasmid, and co-expressed the proteins in E coli (see Methods and FIG. 7-8). Cells were lysed, and soluble and insoluble fractions separated and analyzed by SDS-page. Insoluble fractions with bands for both proteins were examined by negative stain electron microscopy (EM). Of the designs, the most clear lattice assembly was observed for design #13 which formed an extended hexagonal lattice (FIG. 1d, top left panel; see FIG. 9, Table 1 for results on all designs). Design #13 belongs to the p6m symmetry group and is composed of D3 and D2 homooligomers (in the following, we refer to these as components A and B, respectively). FIG. 1.d top right panel shows that the computational design model is superimposable on the averaged EM density, suggesting the designed interface drives assembly of the intended target array geometry.

TABLE 1
Designed and native protein sequences

1d2t
LALVATGNDTTTKPDLYYLKNSEAINSLALLPPPPAVGSIAFLNDQAMYE
QGRLLRNTERGKLAAEDANLSSGGVANAFSGAFGSPITEKDAPALHKLLT
NMIEDAGDLATRSAKDHYMRIRPFAFYGVSTCNTTEQDKLSKNGSYPSGH
TSIGWATALVLAEINPQRQNEILKRGYELGQSRVICGYHWQSDVDAARVV
GSAVVATLHTNPAFQQQLQKAKAEFAQHQK (SEQ ID NO: 48)
A
MGHHHHHHGG
LALVATGNDTTTKPDLYYLKNSEAINSLALLPPPPAVGSIAFLNDQAMYE
QGRLLRNTERGKLAAEDANLSSGGVANAFSGAFGSPITEKDAPALHKLLT
NMIEDAGDLATRSAKDHYMRIRPFAFYGVSTCNTTEQDKLSKNGSYPSGH
TSIGWATALVLAEINPORQNEILKRGYELGQSRVICGYHWQSDVDAARVV
GSAVVATLHTNPEFQAQLIKAKIEFKQHQK EL (SEQ ID NO: 49)
1tk9
MSLINLVEKEWQEHQKIVQASEILKGQIAKVGELLCECLKKGGKILICGN
GGSAADAQHFAAELSGRYKKERKALAGIALTTDTSALSAIGNDYGFEFVF
SRQVEALGNEKDVLIGISTSGKSPNVLEALKKAKELNMLCLGLSGKGGGM
MNKLCDHNLVVPSDDTARIQEMHILIIHTLCQIIDESF (SEQ ID NO:
100)
B
MG
SLITLVELEWLEHQLIVQLSERLKGQIAKVGELLCECLKEGGKILICGNG
GSAADAQHFAAELSGRYKKERKALAGIALTTDTSALSAIGNDYGFEFVFS
RQVEALGNEKDVLIGISTSGKSPNVLEALKKAKELNMLCLGLSGKGGGMM
NKLCDHNLVVPSDDTARIQEMHILIIHTLCQIIDESF LEHHHHHH
(SEQ ID NO: 50)

Protein sequence of A and B components and of the native protein models (1d2t→A, 1tk9→B). To simplify purification we added a 6×His tags to each component and NcoI/XhoI are appended as part of the cloning process. Design mutations are indicated in bold.

To determine whether co-assembly occurs in vivo or after lysis, we genetically fused superfolder green fluorescent protein (GFP) to the N-terminus of component A (AGFP) (FIG. 1.c). Cells were lysed and cell pellets imaged by negative stain EM to verify assembly is not obstructed by the added domain. FIG. 1.d lower left and lower right panels show the formation of hexagonal arrays, with the GFP appearing as roughly spherical density near the trimeric hubs consistent with the design model. We used confocal microscopy to image cells expressing either only AGFP (FIG. 1.e, right panel) or both AGFP and B(FIG. 1.e, left panel) components. Cells expressing only AGFP had a uniform GFP signal as expected for a freely diffusing soluble protein. In contrast, GFP fluorescence was localized to specific regions in cells expressing both components (AGFP+B), suggesting that array assembly occurs in living cells.

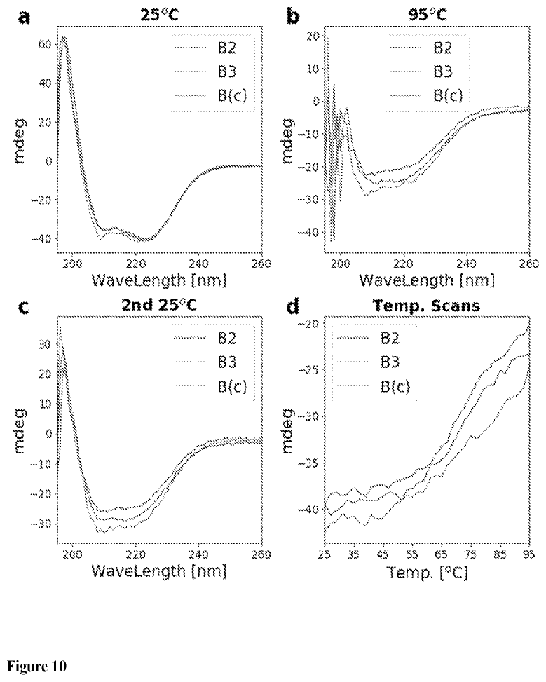

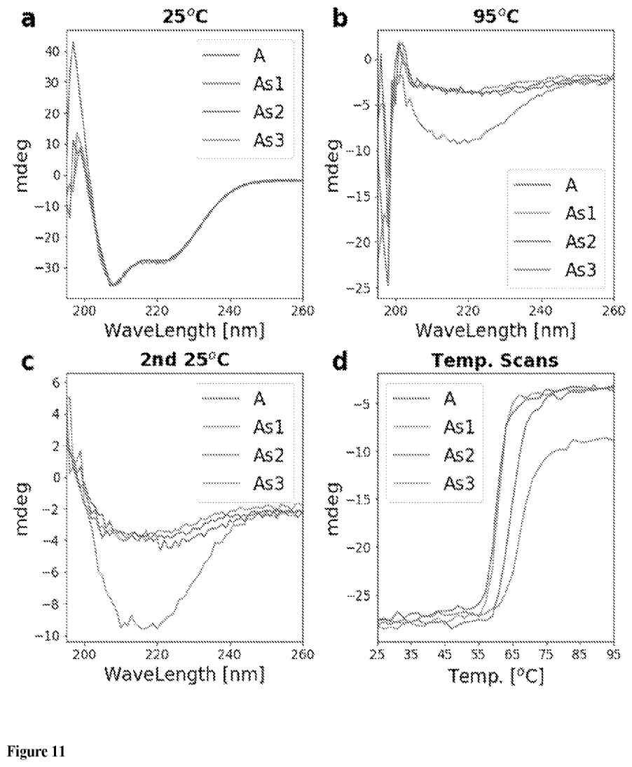

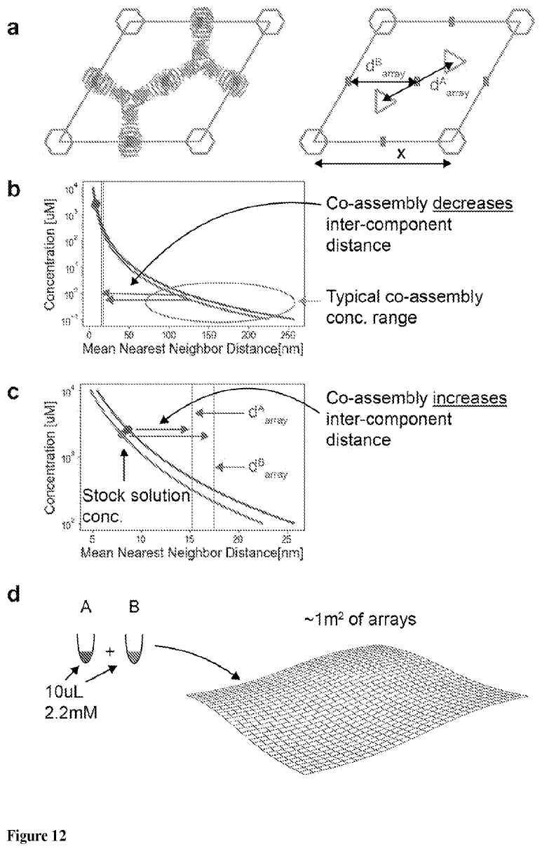

A notable advantage of two-component materials is that if the components are soluble in isolation, co-assembly can in principle be imitated by mixing. This is important for unbounded (i.e. not finite in size) crystalline materials which typically undergo phase separation as they crystallize, complicating the ability to work with them in solution. A measure of binary system quality is the ratio between the maximum value in which either component remains individually soluble to the minimal concentration at which they co-assemble when mixed; the higher this ratio, the easier to prepare, functionalize, and store the components in ambient conditions. To evaluate this ratio, which we refer to as SACA (Self-Assembly to Co-Assembly) we separately expressed and purified the A and B components. We found the A component to be quite soluble with the expected molecular weight by SEC-MALS, but component B precipitated overnight. To improve solubility of A and B components we stabilized both using evolution guided design. We found that both components could then be stored at concentrations exceeding 2 mM at room temperature for an extended duration (see methods and Table 2, FIGS. 10-11 for CD results, and 14a for SAXS of the individual components). While stored individually. A and redesigned B components do not aggregate, but they co-assemble at concentrations as low as −10 nM, thus for this system SACA>10⁵. In fact, this value is so high, that upon assembly from stock solutions at mM concentrations the distance between each component increases (within the plane) to about twice the estimated mean nearest neighbor distance (see FIG. 12 for further discussion), and the solution instantaneously jellifies (See S1 movie S2). With a binary system established, in which components can be modified both by genetic fusion and post-translation peptide fusion (see below), it becomes straightforward to form arrays with combinatorially increased functionalities: arrays can be made from mixtures of Ax1+Ax2+ . . . +Axn with Bx1+ . . . +Bxn. Specific examples where incorporating multiple functions is useful are described below.

TABLE 2
Sequences of the B component stabilized versions

i13B
MGSLITLVELEWLEHQLIVQLSERLKGQIAKVGELLCECLKKGGKILICG
NGGSAADAQHFAAELSGRYKKERKALAGIALTTDTSALSAIGNDYGFEFV
FSRQVEALGNEKDVLIGISTSGKSPNVLEALKKAKELNMLCLGLSGKGGG
MMNKLCDHNLVVPSDDTARIQEMHILIIHTLCQIIDESFLEHHHHHH
(SEQ ID NO: 107)
Di13B1
MGSLITLVELEWLEHQLIVQLSERLKGQIAKVGELLCECLKNGGKILICG
NGGSAADAQHFAAELSGRYKKERKALAGIALTTDTSALSAIGNDYGFEFV
FSRQVEALGNEGDVLIGISTSGKSPNVLEALKKAKELNMLCLGLSGKGGG
KMNKLCDHNLVVPSDDTARIQEMHILIIHTLCQIIDEAFLEHHHHHH
(SEQ ID NO: 108)
Di13B2
MGSLITLVELEWLEHQLIVQLSERLKGQIAKVGELLCECLKNGGKILICG
NGGSAADAQHFAAELSGRYKKERKALAGIALTTDTSALSAIGNDYGFEFV
FSRQVEALGNEGDVLIGISTSGKSPNVLEALKKARELNMLCIGLSGKGGG
KMNDLCDHNLVVPSDDTARIQEMHILIIHTLCQIIDEAFLEHHHHHH
(SEQ ID NO: 109)
Di13B3
MGSLITLVELEWLEHQLIVQLSERLKGQIAKVGELLCRALKNGGKILICG
NGGSAADAQHFAAELSGRYKKERKALAGIALTTDTSALSAIGNDYGFEFV
FSRQVEALGNEGDVLIGISTSGKSPNVLEALKKARELGMLCIGLSGKGGG
KMNDLCDHCLVVPSDDTARIQEMHILIIHTLCQIIDEAFLEHHHHHH
(SEQ ID NO: 110)
Di13B4
MGSLITLVELEWLEHQLIVQLSERLKGQIAKVGELLCRALKNGGKILICG
NGGSAADAQHFAAELSGRYKKERKALAGIALTTDTSALSAIGNDYGFEYV
FARQVEALGNEGDVLIGISTSGKSPNVLEALKKARELGMLCIGLSGKGGG
KMNDLCDHCLVVPSDDTARIQEMHILIIHTLCQIIDEAFELHHHHHH
(SEQ ID NO: 111)

To improve the protein stability and potentially at the same time expression levels we used the PROSS server. Because at that time the protocol did not include symmetry design we optimized only the monomeric interactions by restricting from design all the residues in proximity to both the intra- and inter-homooligomer interfaces (the first are the interfaces forming the homooligomer, and the second are the arrays forming interfaces). Sequences of the design component B and 4 stabilized versions are shown. Mutations that were introduced by the stabilization protocol are indicated in bold. The protocol allows different degrees of sequence manipulations, i.e., number of introduced stabilizing mutations. The higher the number of mutations the better is the expected result, however, also the higher is the risk to damage the overall protein. While the original B component design was aggregating within a day in room temp versions B2 to B4 were all highly stable in room temp, and could be stored at over 2 mM for periods of months. Following the stabilization process we predominantly use the B2 version.

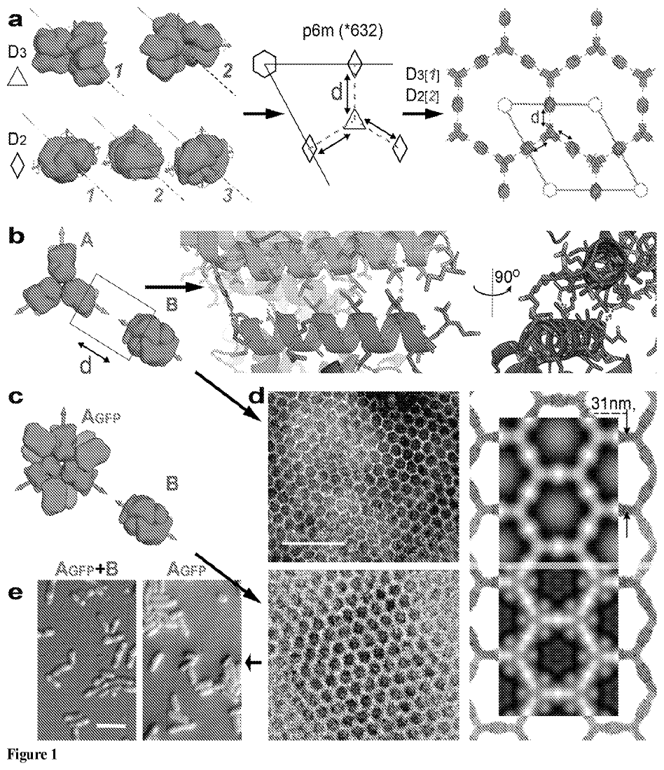

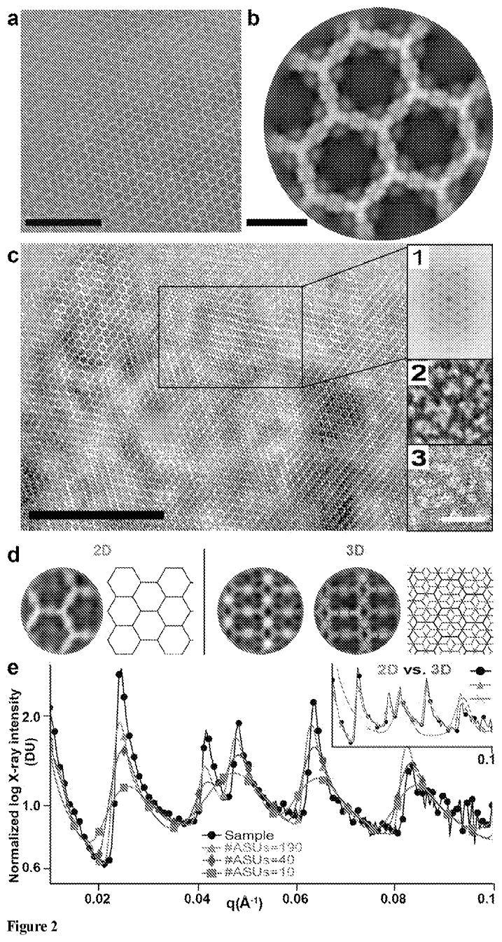

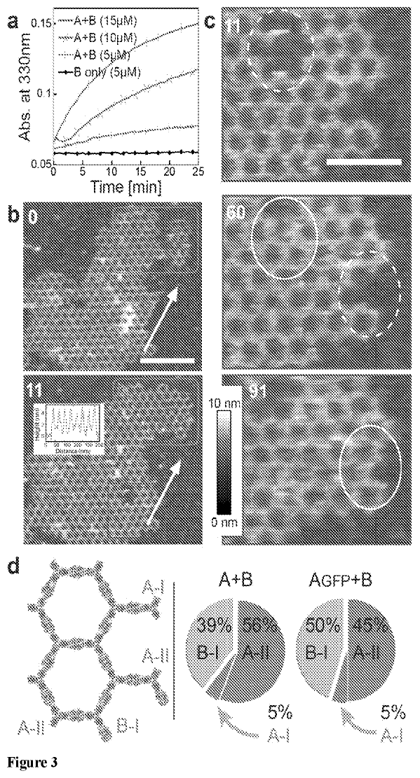

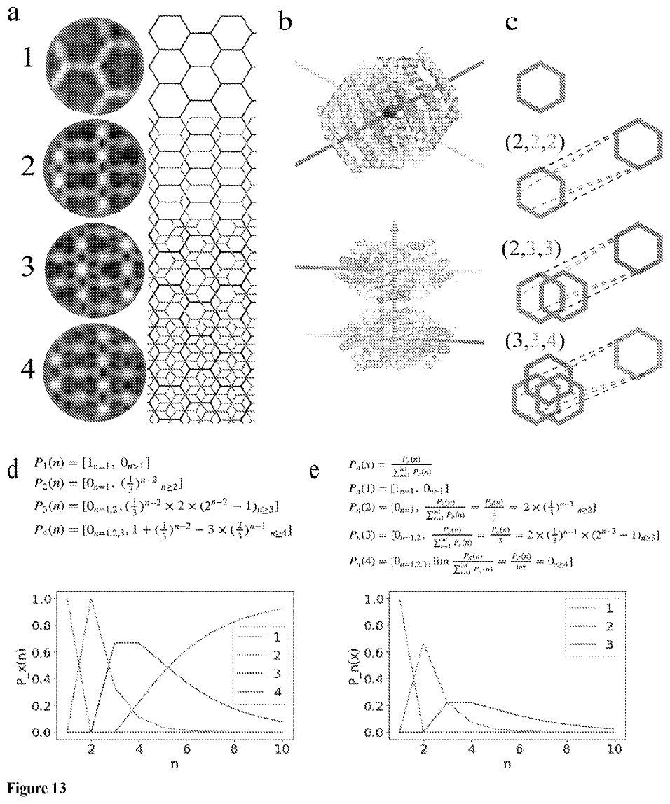

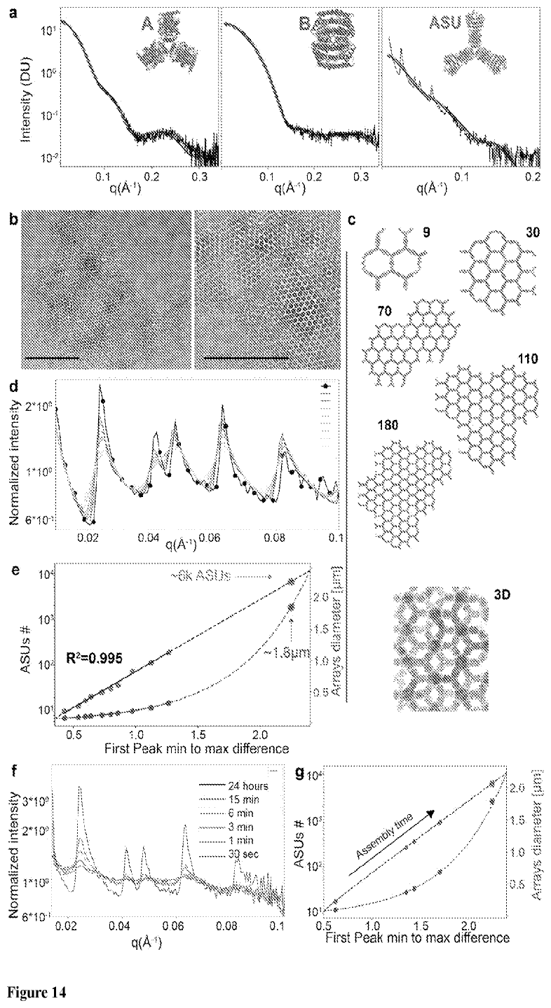

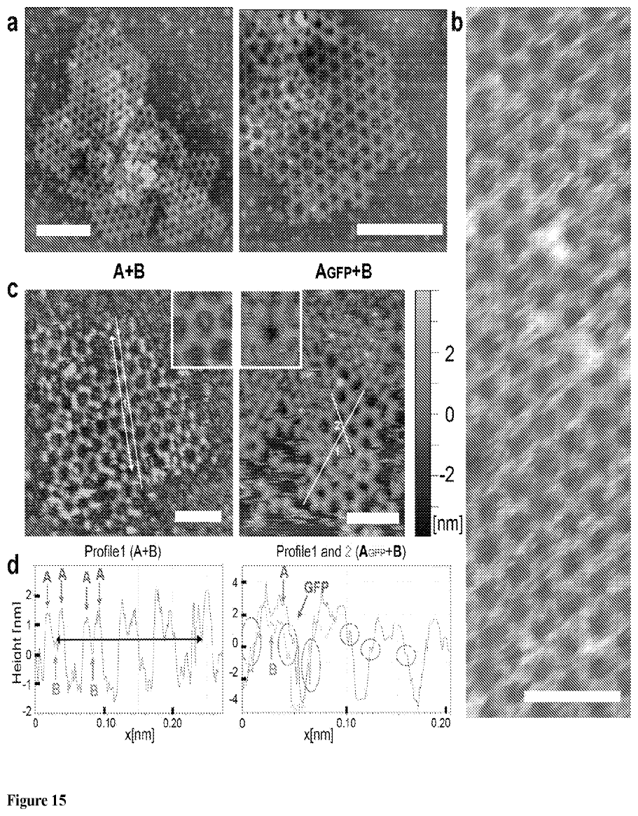

Upon mixing the two purified proteins in vitro at equimolar concentrations, even larger and more regular hexagonal arrays were formed compared to in vivo assembly in bacteria (FIG. 2a,c vs. FIG. 1d). The arrays survive transferring to the TEM grid, incubation with negative stain etc, despite being only ˜4 an thick (see design model and AFM imaging in the inset to FIG. 3.b lower panel and FIG. 22.c-d), suggesting a considerable in-plane strength. No assembly was observed with either component alone (see FIG. 2.c insets 2 and 3 for TEM, FIG. 3.a black curve for light scattering). The density is closely superimposable on the design models, with the outlines of both components evident (FIG. 2.b), suggesting the structure of the material is very close to the computational model. Some stacking of two, three, and four layers of the arrays was observed in EM (FIG. 2.c) with nearly crystalline order and a small, discrete number of symmetry preserving packing arrangements (FIG. 2.d right side panel and FIG. 13) consistent with a single preferred alignment between layers (see FIG. 13 for further discussion). To assess whether such stacking occurs in solution and to evaluate the long range order, we used solution x-ray scattering (SAXS). Scattering rings appear in SAXS data at Bragg peaks consistent with P6 symmetry, and unit cell spacing of 303 Å (data not shown) in close agreement with the designed 2D array model (310 Å), and AFM data (310 Å) (FIG. 14c,d), but not with the 3D stacked arrangement (inset to FIG. 2.e with computational models in 14c). The agreement between the experimental SAXS profile and SAXS profiles computed from the design model increases with increasing numbers of subunits included in the model (FIG. 14c,e), suggesting that in solution the arrays are at least 1.8 μm in diameter.

We investigated the kinetics and mechanism of in vitro assembly by mixing the two components and then monitoring growth in solution by light scattering, and on a substrate by AFM (FIG. 3). Upon mixing the two components in solution at micromolar concentrations, lattice assembly occurred in minutes with concentration-dependent kinetics (FIG. 3.a). The hexagonal lattice could be readily visualized by AFM, and the pathway of assembly assessed by in situ AFM imaging at different time points (FIG. 3.b-c). The designed 2D material exhibited self-healing: cracked edges reform (FIG. 3.b red square) and point defects and vacancies in the interior of the lattice evident at early time points were filled in at later time points (FIG. 3.c, white circles). To determine whether the rate-limiting step in growth is initiation or completion of hexagonal units, we counted the numbers of each of the possible edge states in a set of AFM images. The results show that A units bound to two B units—designated A-II sites—comprise the most stable edge sites, while A units with just one neighboring B unit—designated A-I sites—were the least stable, occurring far less often than exposed B-I sites (FIG. 3.d). The results imply that attachment of a B unit to an A-I site to create a (most) stable A-II site is rate limiting during assembly. Assuming the observed percentages of occurrence p(i) represent an equilibrium distribution, we can estimate the relative free energies ΔG(i-j) of any two sites from ΔG(i-j)=−kTln(pᵢ/pⱼ), from which we obtain ΔG(A-II−A-I)=−6.2 kJ/mol, ΔG(B-I−A-I)=−5.4 kJ/mol, and ΔG(A-II−B-I)=−0.9 kJ/mol (See FIG. 15 for measurement analysis and additional results on assembly using the GFP-modified version of A). A preliminary SAXS study shows rapid formation of Bragg peaks at positions consistent with the computed model (see FIG. 14f, g and methods) from which arrays dimensions are estimated at 0.4 μm in diameter within the first 2 minutes after mixing the components (at 10 μM) and at 0.7 μm within 6 min. This suggests that solution SAXS measurements enable a thorough kinetics study and construction of phase diagrams of macroscale 2D binary systems.

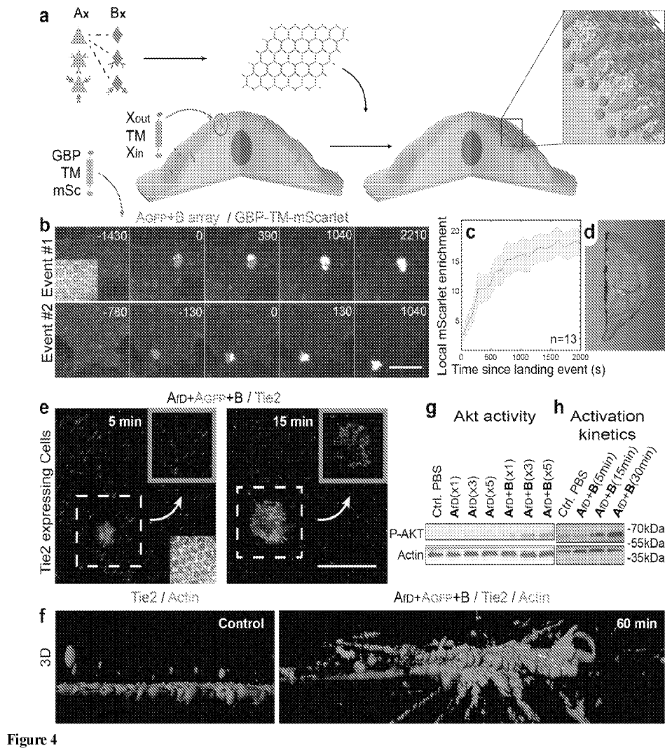

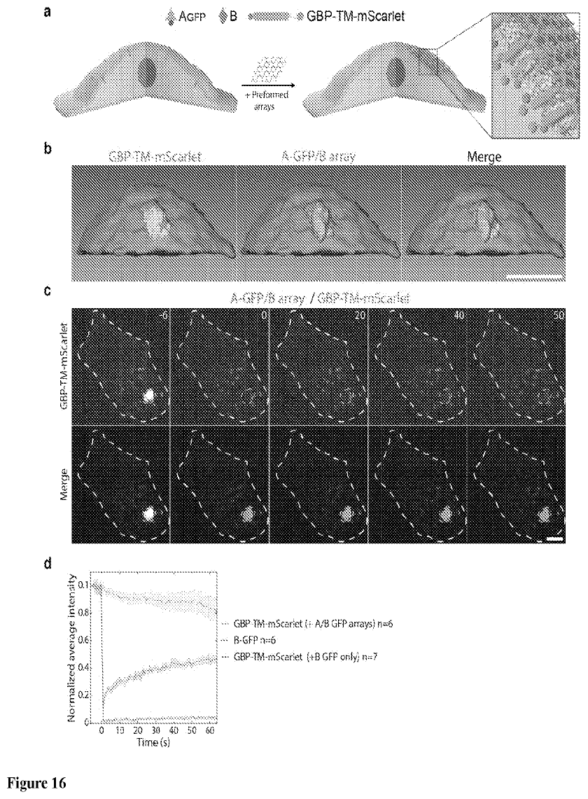

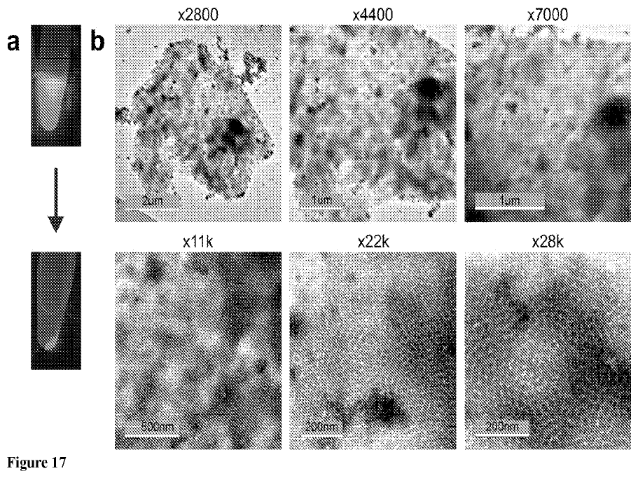

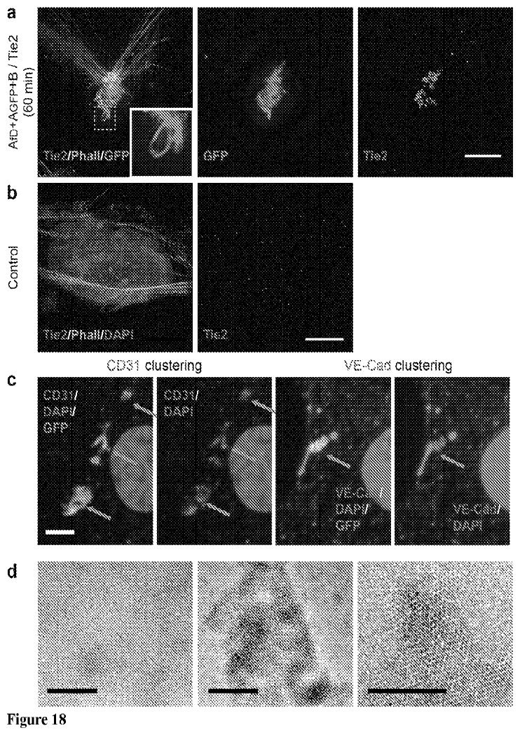

We next investigated if preformed arrays could cluster transmembrane receptors on living cells (FIG. 4). In contrast to antibodies, which have been used extensively to crosslink cell surface proteins, the arrays provide an extremely large number of attachment sites in a regular 2D geometry. To measure the clustering kinetics, we stably expressed a model receptor composed of a transmembrane segment (TM) fused to an extracellular GFP binding domain (GBP, GFP Binding Peptide) and an intracellular mScarlet (noted GBP-TM-mScarlet; FIG. 4a). In the absence of arrays, the mScarlet signal was diffuse, but when a preformed AGFP+B array landed onto the cells, mScarlet signal clustered under the GFP signal over a time scale of roughly 20 minutes (FIG. 4.b-c, see also FIG. 4.d, 16 and FIG. 17 for confirmation by EM that the purified arrays employed in these conditions indeed display the characteristic hexagonal lattice). Fluorescence Recovery After Photobleaching (FRAP) analysis further showed that once localized, the receptors remain largely held in place by the arrays (FIG. 16.c-d). To determine if the patterned and highly multivalent interactions between the arrays and cell surface receptors can induce a downstream biological signal, we targeted the Tie-2 receptor. Using the SpyCatcher™-SpyTag™ (SC-ST) conjugation system, we fused the F domain of the angiogenesis promoting factor AngI, the ligand for the Tie-2 receptor, to A_{SC}, a modified A component having SpyCatcher™ genetically fused to its N-terminus (AfD). Following incubation of Human Umbilical Vein Endothelial Cells (HUVECs) with pre-assembled arrays displaying both Ang1 and GFP (AfD+AGFP+B), green patches were observed at the plasma membrane, which extensively clustered the endogenous Tie-2 receptors (FIG. 4.e, and FIG. 1g for further examples, controls, and TEM characterisation), with kinetics comparable to our model transmembrane protein (FIG. 4.e). Because we adjusted the amount of arrays to have only a small number (0-2) associated with any individual cell, and the arrays are labeled, the effects of large scale receptor clustering on downstream protein recruitment events can be investigated in detail. We used super-resolution microscopy to investigate the effects on the cytoskeleton, and observed extensive remodeling of the actin cytoskeleton underneath the Tie-2 receptors after 60 minutes (FIG. 4.f), which could reflect adherens junction formation (see FIG. 18.c for other markers). Western blot analysis showed that the Ang1 arrays, but not the individual functionalized array component, induces signalling through AKT (FIG. 4.g, h for kinetics), suggesting that the Ang1 arrays indeed induce their expected physiological signalling pathway.

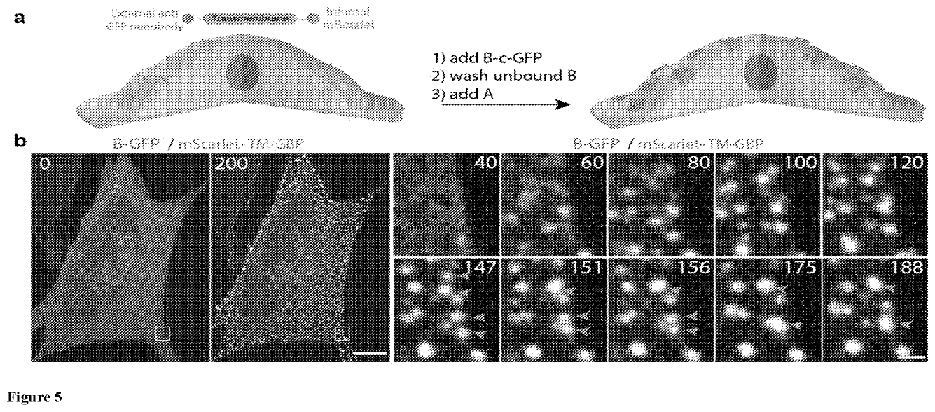

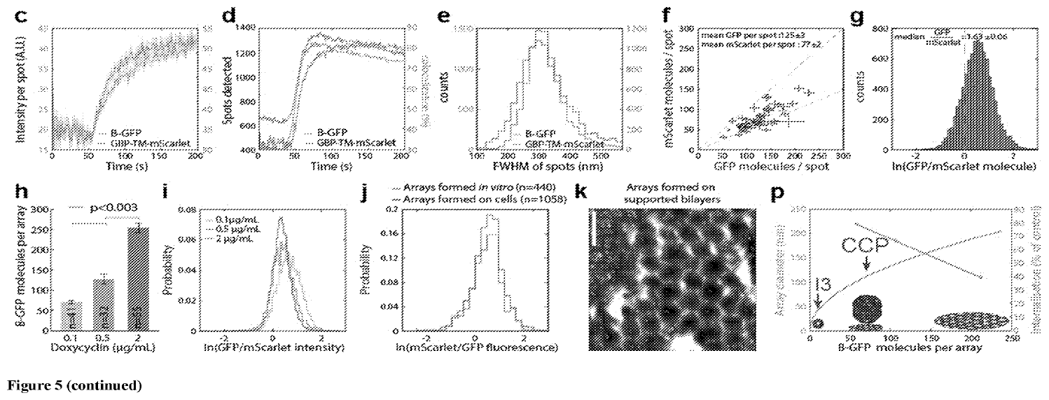

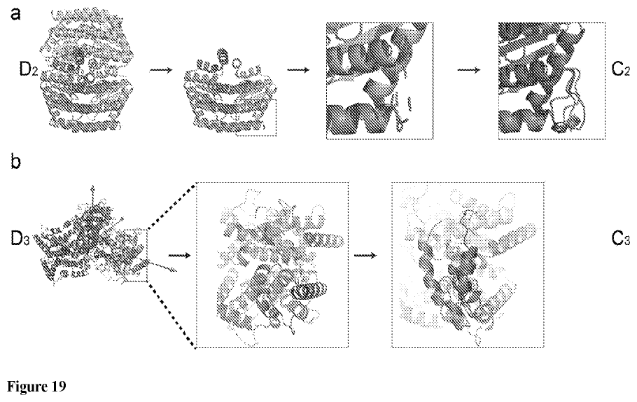

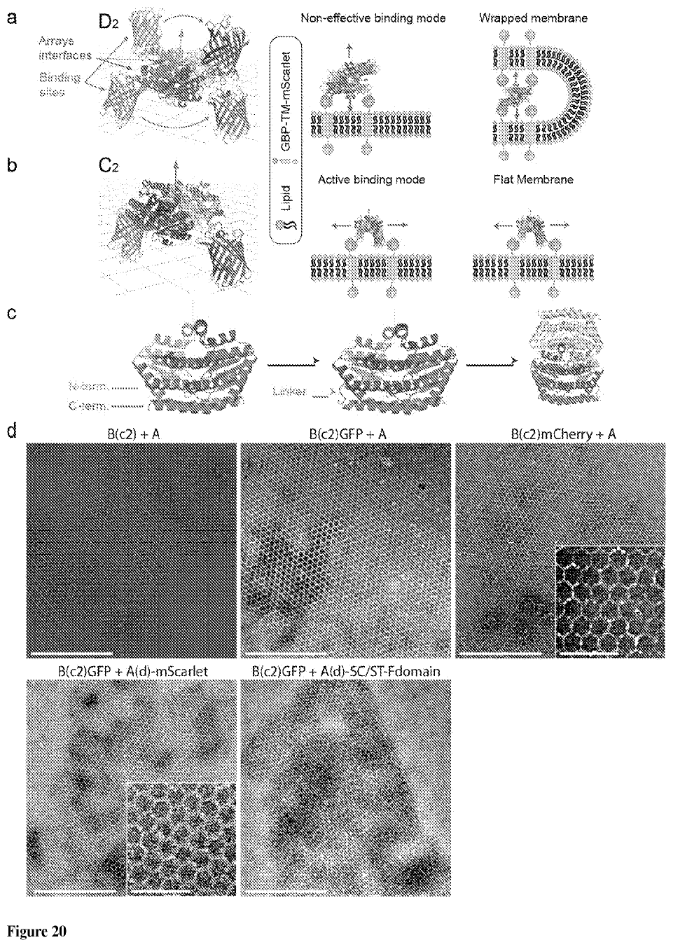

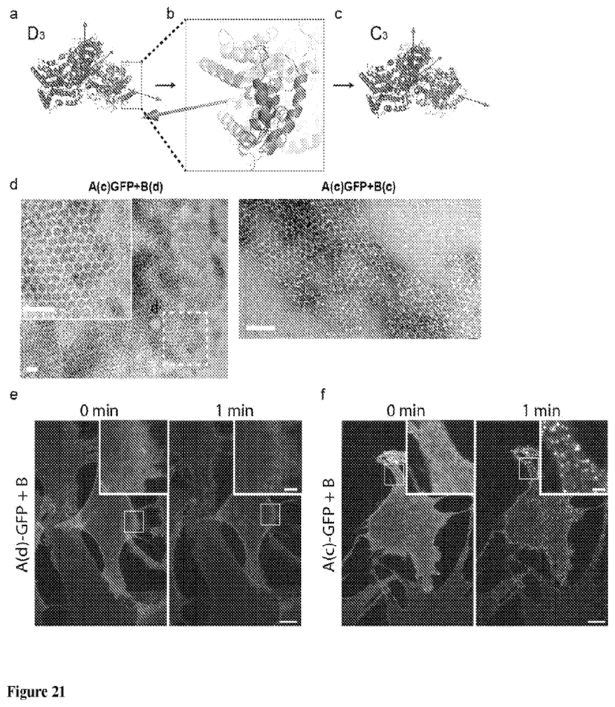

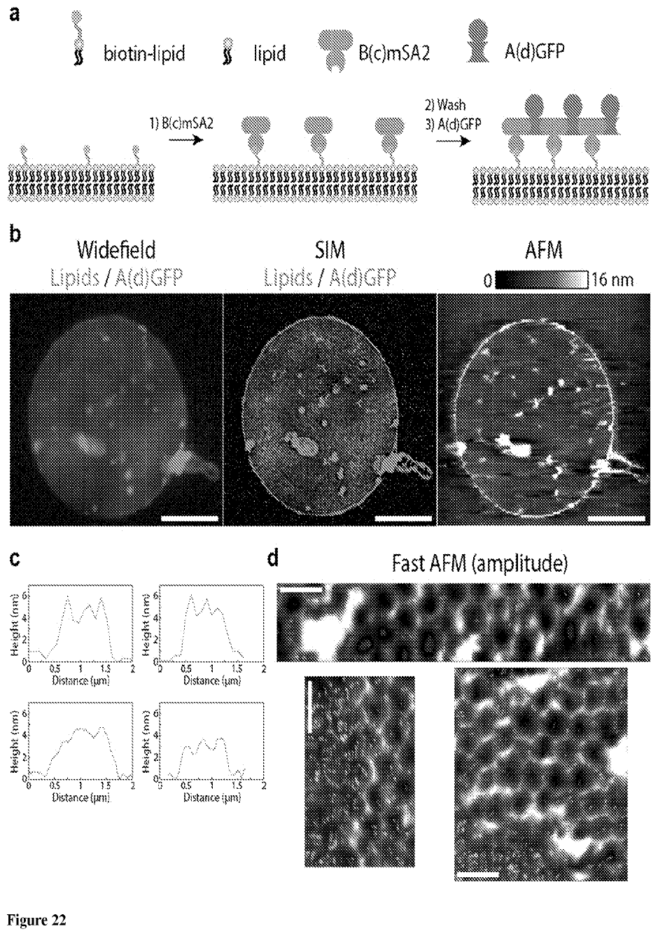

Taking advantage of the two-component nature of the material, we sought to speed up assembly kinetics and homogeneity of clustering by first saturating membrane receptors with one component, then triggering assembly on cells with the second (FIG. 5.a). We found that the original dihedral building blocks were not well suited for this task, most likely because of their inherent equal distribution of binding sites on both of their sides: the membrane can wrap around to bind both sides, thereby blocking assembly (se FIGS. 19 and 20a,b for further discussion). We thus devised cyclic pseudo-dihedral versions of the A and B components (referred to as A(c), B(c) and A(c)GFP, B(c)GFP) by introducing linkers between positions near the C-terminus of one subunit and positions near the N-terminus of another (see FIGS. 19-21 and Tables 3 and 4). We found by AFM characterization of arrays grown on supported lipid bilayers that first tethering one cyclic component then adding the other leads to formation of 2D hexagonal arrays nearly identical to those formed in solution (FIG. 5.k and 22 and methods). Likewise, both cyclic components enabled array formation onto cells expressing the GBP-TM-mScarlet when incubated first with the cyclic component, and then after washing, the second component (FIG. 5.a-d for B(c)GFP, then A and S16.f A(c)GFP, then B).

TABLE 3
B component desymmetrization linkers list

| Construct name | B2 - linker - B4 |
|---|---|
| 1 Di13_B2L1B4 | IDEAF GGGSGGS SLITL |
| | (SEQ ID NO: 112) |
| 2 Di13_B2L2B4 | IDEAF GGGKDRNG GSLIT |
| | (SEQ ID NO: 113) |
| 3 Di13_B2L3B4 | IDEAF TGDAGET SLITL |
| | (SEQ ID NO: 114) |
| 4 Di13_B2L4B4 | IDEAF GGETSSKQD LITLV |
| | (SEQ ID NO: 115) |

Linker inserted (bold letters) between the C-terminal of one monomer (N-terminal to linker) and the N-terminal of another monomer (C-terminal to linker). Note the N-terminal of the second monomer was trimmed in some of the cases. Construct number 2 was best behaving and verified under TEM to form the expected hexagonal geometry with the dihedral A components with or without the addition of GFP/mcherry labels fused at the C-terminus (see FIG. 20.d ).

TABLE 4
A component desymmetrization linkers list
Construct name A - linker - As3

```
  1 Di13A_S1L12_As3_n0 KQHQK FRQQPPPPQQSG
GLALVATGNDATTKPDLYYLKNSEAINSL (SEQ ID NO: 116)
  2 Di13A_SIL14_As3_n1 KQHQK DKTPEDSTRSEYKG
GLALVATGNDATTKPDLYYLKNSEAIN (SEQ ID NO: 117)
  3 Di13A_S8L13_As3_n2 KQHQK SEPQEVSETQEVP
GNDATTKPDLYYLKNSEAINSLALLPPP (SEQ ID NO: 118)
  4 Di13A_S18L12_As3_n3 KQHQK ESTKSWPPTSPA
YYLKNSEAINSLALLPPPPAVGSIAFLND (SEQ ID NO: 119)
  5 Di13A_S14L10_As3_n4 KQHQK QQQEERQTDK
KPDLYYLKNSEAINSLALLPPPPAVGSIAFL (SEQ ID NO: 120)
  6 Di13A_S18L10_As3_n5 KQHQK DSESSGEPGA
YYLKNSEAINSLALLPPPPAVGSIAFLNDQA (SEQ ID NO: 121)
  7 Di13A_S14L13_As3_n6 KQHQK SRDDDKGAKHKPK
KPDLYYLKNSEAINSLALLPPPPAVGSI (SEQ ID NO: 122)
  8 Di13A_S8L18_As3_n7 KQHQK SDSKEEEKKKSSDNSSTP
GNDATTKPDLYYLKNSEAINSLA (SEQ ID NO: 123)
  9 Di13A_S1L18_As3_n8 KQHQK KPDERSSSKKEEDKKDRG
GLALVATGNDATTKPDLVYLKNS (SEQ ID NO: 124)
10 Di13A_S14L11_As3_n9 KQHQK GSGSGSGSGSG
KPDLYYLKNSEAINSLALLPPPPAVGSIAF (SEQ ID NO: 125)
11 Di13A_S8L13_As3_n10 KQHQK GSGSGSGSGSGSG
GNDATTKPDLYYLKNSEAINSLALLPPP (SEQ ID NO: 126)
12 Di13A_S1L14_As3_n11 KQHQK GSGSGSGSGSGSGS
GLALVATGNDATTKPDLYYLKNSEAIN (SEQ ID NO: 127)
Linker inserted (bold letters) between the C-terminal of one monomer (N-terminal to linker) and various truncations of the N-terminal of a monomer version As3 (C-terminal to linker). Constructs name nomenclature Di13 A for the first monomer, SX: X is the number of residues truncated of the second monomer N-terminus, LX: X is the linker length (residues
number), and As3 - the stabilized monomer version used as the second monomer. Construct number 3 was best behaving and verified under TEM to form the expected hexagonal geometry both when mixed with dihedral B or cyclic B components (see FIG. 20d).
```

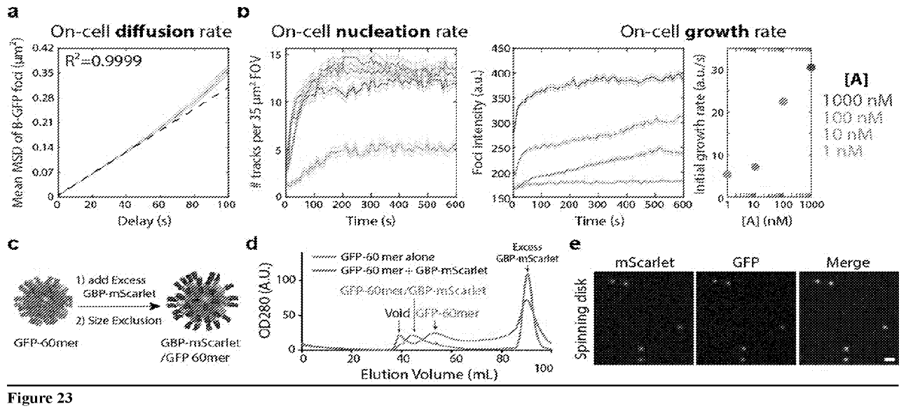

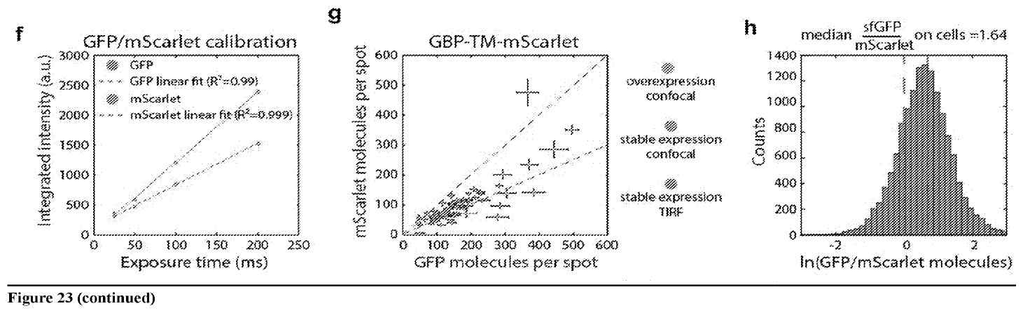

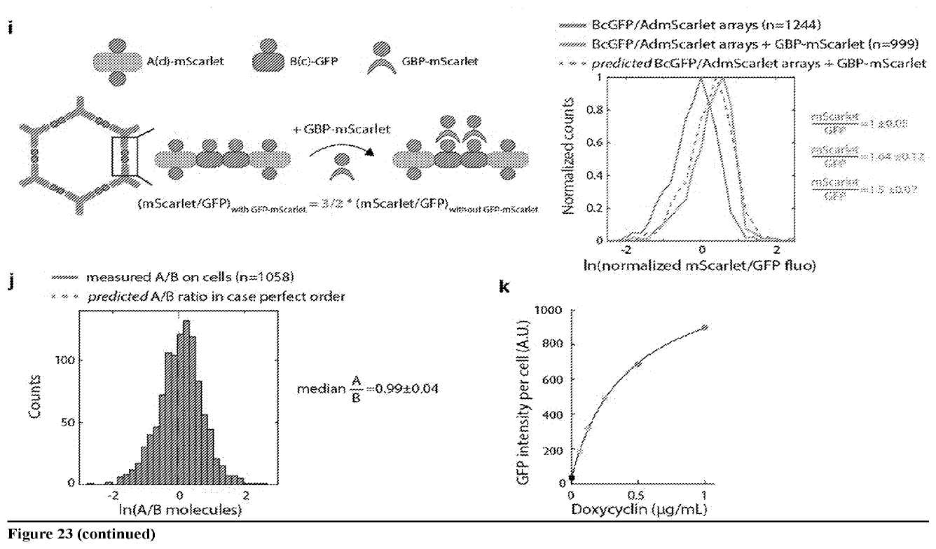

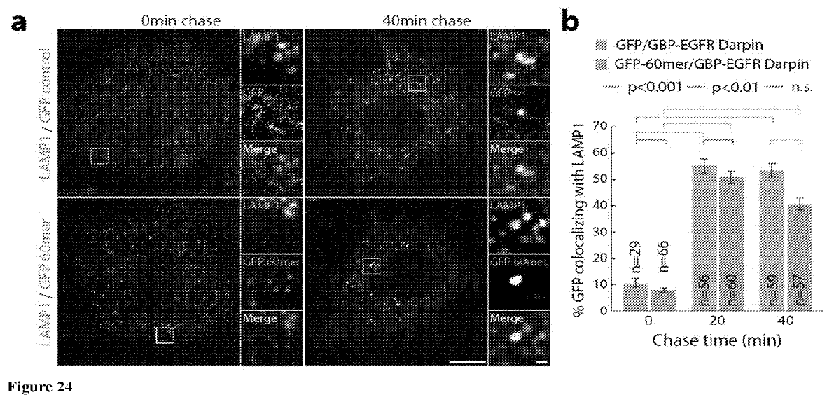

Array formation on cells using this method was fast (steady state reached in =20s) and colocalizing mScarlet™ patches appeared synchronously with GFP-positive patches, indicating that receptor clustering was fast as well (FIG. 5.b, FIG. 5.c-d for quantification). These diffraction-limited arrays eventually stop growing, likely due to the lack of available transmembrane-anchored B(c)GFP. Instead, they slowly diffuse (D=0.0005 μm²/s, FIG. 23a), and eventually merge into larger arrays (FIG. 5.b arrows, see also FIG. 5.d for quantification). Receptor clustering by array assembly onto cells was at least one order of magnitude faster than with preformed arrays (20 min in FIG. 4.b, c compared to 20s in FIG. 5.b, d), and fully inducible (nothing happens until A is added), synchronized (all arrays appear at the same time within the cell and between cells, see FIG. 5.b, d) and homogenous (all arrays have similar diffraction limited size; see FIG. 5.e). On cell assembly dramatically improved clustering synchronisation compared to preformed arrays: the 760 clusters in FIG. 5.d appeared within =15s, while there was a delay time of 980±252s (mean±SEM) between the onset of receptor clustering for the 13 events in the dataset presented in FIG. 4.c. The rate of array nucleation on cells decreased with decreasing concentrations of A in solution (FIG. 23.b, rapid increase in fluorescence at short times). In addition, as expected for a material formed from equal numbers of two components, there was also a strong dependence of the array growth rate on the concentration of A: higher concentrations of A increased the initial growth rate, but this rate decays faster over time likely due to the saturation of all available B components (FIG. 23b).

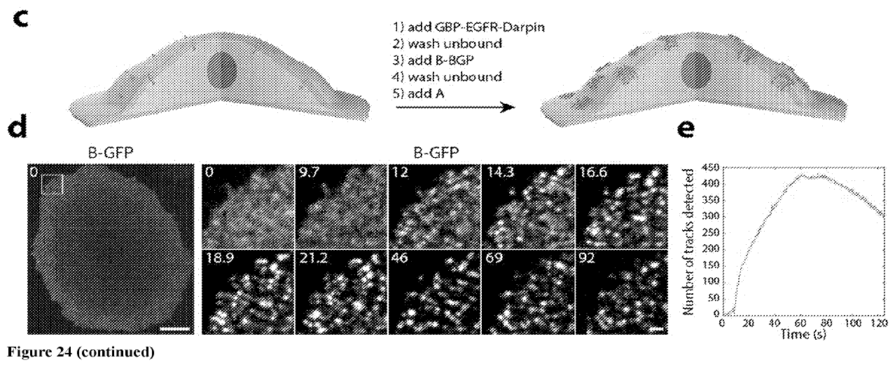

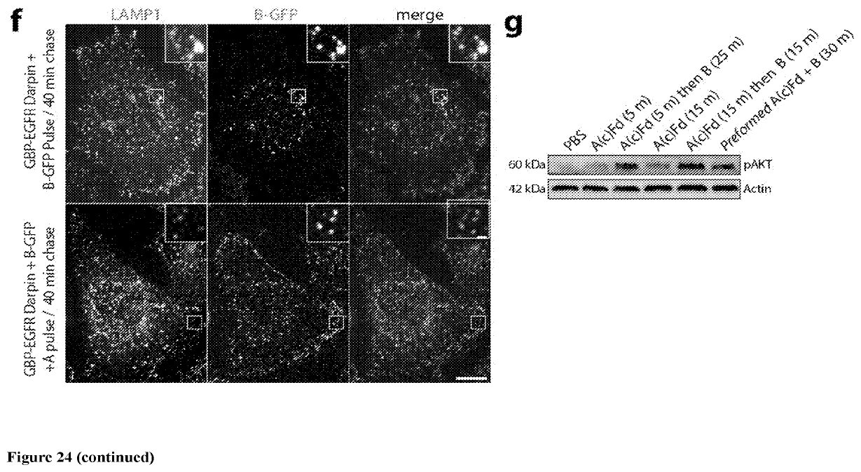

To evaluate how many molecules were clustered per array, we adapted our previously described microscope calibration nanocages to two colors (see methods and FIG. 23c-f) and found that each diffraction-limited array contained on average 125±3 GFP and 77±2 mScarlet molecules (FIG. 5.f). This GFP/mscarlet ratio per array was remarkably similar not only within the same cell, but also between cells, suggesting that all arrays are virtually identical within the cell population and that the number of receptors clustered scales homogeneously with the array size (FIG. 5.f g and also FIG. 23.g-h for a larger range of array size). The median GFP/mscarlet ratio of 1.63±0.06 (FIG. 5.g) is within the expected [12] range, corresponding to either 1 or 2 GBP-TM-mScarlet bound per B(c)GFP dimer. Tie2 signaling experiments analogous to those in FIG. 4 showed that arrays formed on cells are functionally equivalent to preformed arrays as they elicit at least as much downstream Akt phosphorylation (FIG. 24g), consistent with large numbers of array components and hence underlying receptors being clustered.

We explored tuning the final size of the array by tuning the density of receptors at the cell surface. We used a doxycycline-inducible promoter to control the expression of the synthetic membrane protein and thus its density at the cell surface (FIG. 23.k). This provided control over the final size of arrays assembled at the cell surface (FIG. 5.h), while the clustering efficiency, that is, the GFP/mScarlet ratio, remained similar (FIG. 5.i). Array size can also be modulated by varying the concentration of A while keeping the receptor density constant (FIG. 23b).

We then investigated whether the lattice order (FIGS. 1-3) was conserved when arrays were assembled onto cells. We compared the mScarlet/GFP fluorescence ratio of B(c)GFP/A(d)mScarlet arrays formed either in vitro, or on cells expressing the transmembrane domain fused only to the extracellular GBP (GBP-TM). As shown in FIG. 5.j, the mScarlet/GFP ratio was remarkably similar between arrays assembled in vitro or onto cells, suggesting comparable degree of order (mScarlet/GFP fluorescence ratio of 1.45±0.07 for in vitro versus 1.48±0.06 for cells, see also FIG. 20.d for verification of the order of preformed B(c)GFP/A(d)mScarlet arrays and 23i for verification that the mScarlet/GFP fluorescence ratio is a valid measure of bulk order, as it varies as predicted when mScarlet molecules are added to B(c)GFP/A(d)mScarlet arrays via nanobodies). We independently confirmed these results by directly evaluating the A/B ratio of arrays generated on cells using our calibrated microscope and obtained A/B=0.99±0.04 (FIG. 23j and methods). This is in good agreement with our findings by fast AFM that arrays assembled on supported bilayers are indeed 2D, single layered and ordered (FIG. 5.k, FIG. 22 and methods).

Following ligand-induced oligomerization, numerous receptors, such as the Epidermal Growth Factor Receptor (EGFR), are internalized by endocytosis and degraded in lysosomes as a means to downregulate signalling. It is therefore not a surprise that EGFR oligomerisation agents, such as combinations of antibodies recognizing different epitopes or bivalent heterotypic nanobodies induce rapid EGFR endocytosis and degradation in lysosomes. This rapid endocytosis is not specific to small oligomers, as large 3D oligomers, such as our 60-mer nanocages³⁰ functionalized with EGFR binders, are also rapidly internalised and routed to lysosomes (FIG. 24a, b). This phenomenon has been proposed to lower the efficiency of immunotherapy in in vivo models. We thus wondered if the unique features of our material, namely 2D geometry and large size compared to clathrin coated vesicles, could modulate this effect. We found that arrays are able to cluster endogenous EGFR in HeLa cells with similar kinetics as the GBP-TM-mScarlet construct, suggesting that the fast kinetics seen in FIG. 5.a-i are not due to the properties of this single-pass synthetic model receptor, but are rather a property of the arrays themselves (see FIG. 24c-e). Importantly, we found that while endogenous EGFR bound to dimeric B(c)GFP was rapidly internalized and routed to lysosomes, clustering EGFR by addition of A quantitatively inhibited this effect (FIG. 5.l, m for quantification, and also FIG. 24f for split channel images). This EGFR clustering did not trigger EGF signaling presumably because the distance between receptors in the cluster is longer than within EGF-induced dimers. Notably, the extent of blockage of endocytosis was a function of array size, as tuning down their size using our inducible system relieved the endocytic block (FIGS. 5.n, o for quantification). Several lines of evidence suggest that our designed material assembles in a similar way on cells as it does in vitro. First, AFM showed that assembly of the two components on supported lipid bilayers, using a protocol very similar to that used for on cell assembly, generates single layer arrays with the hexagonal lattice structure nearly identical to those formed in solution (compare FIG. 5.k with FIG. 2.a and 15 with 22). Second, the remarkable homogeneity in the growth rate and size distribution of the arrays assembled on structures resembles ordered crystal growth more than random aggregation. Third, the distribution of the ratio of fluorescence intensities of the two fluorescently labeled array components on cells is the same for preformed arrays with structure confirmed by EM; in contrast disorganized aggregates would be expected to have a wide range of subunit ratios. Fourth, the A/B ratio of arrays generated on cells is close to 1 to 1, consistent with the array structure and again not expected for a disorganized aggregate. While these results suggest that the overall 2D array geometry and subunit stoichiometry are preserved when the arrays assemble on a cell membrane, it will be useful to measure the array defect frequency when technology for structural determination on cells sufficiently improves. This caveat notwithstanding, these results highlight the power of quantitative microscopy to translate structural information from detailed in vitro characterization to the much more complex cellular membrane environment.

Our studies of the interactions of the designed protein material with mammalian cells provides new insights into cell biology of membrane dynamics and trafficking. We observe a strong dependence of endocytosis on array size and on the geometry of receptor binding domain presentation: arrays roughly the size of clathrin coated pits almost completely shut down endocytosis, while smaller arrays, and nanoparticles displaying large numbers of receptor binding domains are readily endocytosed (FIG. 5.p). The mechanism of this endocytic block likely relates to the increased curvature free energy and/or membrane tension and further investigation should shed light on mechanisms of cellular uptake. From the therapeutics perspective, the ability to shut down endocytosis without inducing signaling, as in our EGFR binding arrays, could be very useful for extending the efficacy of signaling pathway antagonists, which can be limited by endocytosis mediated drug turnover. Furthermore, the ability to assemble protein materials around cells opens up new approaches for reducing immune responses to introduced cells, for example for type I diabetes.

The long range almost-crystalline order, tight control over the timing of assembly, and the ability to generate complexity by modulating the array components differentiate the designed two dimensional protein material described here from naturally occurring and previously designed protein 2D lattices. Applied to biology, this new material provides an unprecedented way to rapidly and quantitatively cluster transmembrane proteins, effectively enabling modulating signalling pathways from the outside. In particular, the stepwise assembly approach described here offers a fine level of control to cluster receptors compared to pre-assembled materials or aggregates: not only is receptor density in the clusters fixed at the structural level, but also the fluorescence intensity of the array component can be directly converted into the absolute size of receptor clusters and the number of receptors being clustered, which is useful if the receptors are endogenous cell proteins not fluorescently tagged. We anticipate that these properties, combined with the synchrony of receptor clustering should greatly facilitate the detailed investigation of the molecular sequence of events downstream of receptor clustering. Applied to structural biology, the ability to impose a predetermined order onto transmembrane proteins may help structure determination of those challenging targets using averaging techniques. We furthermore envision multiple ways for these two component bio-polymers to integrate into designed and living materials. For example, as two-component bioinks, adhesive bio-printed scaffolds could remove the need for harmful temperature/UV-curing techniques; conversely, embedding cells secreting designed scaffolds building-blocks could continuously regenerate their extracellular structure or induce its remodelling in response to programmable cues. We expect the methodology developed here, combined with the rapid developments in de novo design of protein building-blocks and quantitative microscopy techniques, will open the door to a future of programmable biomaterials for synthetic and living systems.

### Methods:

### Computational Design

Crystal structures of 628 D2, 261 D3, 63 D4, and 13 D6 dihedral homooligomers with resolution better than 2.5 Å were selected from the Protein Data Bank (PDB) to be used as building blocks (BBs). Combinatorial pairs of BBs were selected such that they afford the two rotation centers required in a selected subset of plane symmetries (P3 m1 [C3-C3], p4m [C4-C4, C4-C2], p6m [C6-C2, C6-C3, C3-C2]). The highest-order rotation symmetry axis of each BB was aligned perpendicular to the plane and an additional 2 fold symmetry axis was aligned with the plane symmetry reflection axis. Preserving these constraints allows positioning the D2, D3. D4, and D6 BBs in 6, 2, 2, and 2 unique conformations, respectively, and results in a total of ˜2.6M unique docking trajectories. In a first iteration Symmetric Rosetta™ Design²⁵ was applied to construct the BBs dihedral homooligomers, position them in the correct configuration in space and slide them into contact, along the plane symmetry group reflection axes. Docking trajectories are discarded if clashing between BBs are detected, if a fraction greater than 20% of contact positions (residues belonging to one BB within 10 Å of their partner BB residues) do not belong to a rigid secondary structure (helix/beta sheet), or if the surface area buried by the formation of the contact is lower than 400 Å². These initial filtering parameters narrow the number of potential design trajectories to approximately 1% of the original trajectories number. In a second iteration, the selected docks (BBs pairs contact orientation) are regenerated by Symmetric Rosetta™ Design, slide into contact and retract in steps of 0.05 Å to a maximum distance of 1.5 Å. For each position, layer sequence design calculations, implemented by a Rosetta™ script,²⁶ are made to generate low-energy interfaces with buried hydrophobic contacts that are surrounded by hydrophilic contacts. Designed substitutions not substantially contributing to the interface were reverted to their original identities. Resulting designs were filtered based on shape complementarity (SC), interface surface area (SASA), buried unsatisfied hydrogen bonds (UHB), binding energy (ddG), and number of hydrophobic residues at the interface core. A negative design approach that includes an asymmetric docking is used to identify potential alternative interacting surfaces. Designs that exhibit a non-ideal energy funnel are discarded as well. Forty five best scoring designs belonging to p3 m1:2, p4m: 10, and p6m: 33, were selected for experiments. Protein monomeric stabilization was done to the D2 and D3 homooligomers of design #13 using the PROSS server (see FIG. 10, 11, and Table 2).

Pyrosetta™³⁵ and RosettaRemodel™³⁶ were used to model and generate linkers to render the D2 and D3 working homooligomers into C2 and C3 (cyclic pseudo-dihedral) homooligomers (see FIG. 19-21 and Tables 3-4 for details and further discussion). Linkers for non-structural fusions, i.e., optical labels and binding sites such as spyTag™/spyCatcher™, were not modeled computationally.

### Expression Construct Generation.

Genes encoding for the 45 pairs were initially codon optimized using DNAWorks™ v3.2.4³⁷ followed by RNA ddG minimization of the 50 first nucleotides of each gene using mRNAOptimiser™³⁸ and Nupack3.2.2 programs (FIG. 7).³⁹ For screening in an in-vivo expression setup, bicistronic constructs were cloned (GenScript®) in pET28b+ (kanamycin resistant), between NcoI and XhoI endonuclease restriction sites and separated by an intergenic region ‘TAAAGAAGGAGATATCATATG’ (SEQ ID NO: 128). For the working design, separately expressing constructs were prepared by polymerase chain reaction (PCR) from sets of synthetic oligonucleotides (Integrated DNA Technologies) to generate linear DNA fragments with overhangs compatible with a Gibson assembly to obtain circular plasmids. Additional labels (His tag, sfGFP, mCherry, mScarlet, spyTag™, spyCatcher™, mSA2, and AVI tag) were either genetically fused by a combination of PCR and Gibson processes or through post expression conjugation using the spyTagr™ spyCatcher™ system²⁹ or biotinylation.⁴² Note that the variant of GFP used throughout the paper, on both A/B components and the 60-mer nanocages is sfGFP (referred to as GFP in the text for simplicity).

The transmembrane nanobody construct (FIG. 4-5) consists of an N-terminal signal peptide from the Drosophila Echinoid protein, followed by (His)₆-PC tandem affinity tags, a nanobody against GFP⁴³ (termed GBP for GFP Binding Peptide), a TEV cleavage site, the transmembrane domain from the Drosophila Echinoid protein, the VSV-G export sequence^{44,45} and the mScarlet protein⁴⁶. The protein expressed by this construct thus consists of an extracellular antiGFP nanobody linked to an intracellular mScarlet by a transmembrane domain (named GBP-TM-mScarlet in the main text for simplicity). This custom construct was synthesized by IDT and cloned into a modified pCDNA5/FRT/V5-His vector, as previously described⁴⁷ for homologous recombination into the FRT site. A version without the mScarlet (GBP-TM) was similarly derived. We also modified the backbone to allow Doxycycline-inducible expression by first replacing the EF1a promoter by Tet promoter, then by making the backbone compatible with the MXS chaining system⁴⁸ and ligating in the CMV::rtTA3 bGHpA cassette.

For the GBP-mScarlet and GBP-EGFR-Darpin fusions, we modified a pGEX vector to express a protein of interest fused to GBP downstream of the Gluthatione S transferase (GST) purification tag followed by TEV and 3C cleavage sequences. We then cloned mScarlet and a published Darpin against EGFR⁴⁹ (clone E01) into this vector, which thus express GST-3C-TEV-GBP-mScarlet and GST-3C-TEV-GBP-EGFR-Darpin fusions, respectively.

### Protein Expression and Purification.

Unless stated otherwise, all steps were performed at 4° C. Protein concentration was determined either by absorbance at 280 nm (NanoDrop™ 8000 Spectrophotometer, Fisher Scientific), or by densitometry on coomassie-stained SDS page gel against a BSA ladder.

For initial screening of the 45 designs for A and B, bicistronic plasmids were transformed into BL21 Star (DE3) E. coli, cells (Invitrogen) and cultures grown in LB media. Protein expression was induced with 1 mM isopropyl β-d-1-thiogalactopyranoside (IPTG) for 3 hours at 37° C. or 15 hours at 22° C., followed by cell lysis in Tris-buffer (TBS; 25 mM Tris, 300 mM NaCl, 1 mM dithiothreitol (DTT), 1 mM phenylmethylsulfonyl fluoride (PMSF), and lysozyme (0.1 mg/ml) using sonication (Fisher Scientific) at 20 W for 5 min total ‘on’ time, using cycles of 10s on, 10s off. Soluble and insoluble fractions were separated by centrifugation at 20,000×g for 30 minutes and protein expression was screened by running both fractions on SDS-PAGE (Bio-Rad) (see FIG. 9) and for selected samples also by negative stain EM. All subsequent experiments done on separately expressed components were performed on (His)6-tagged proteins. Following similar expression protocols (22° C./15 hours) cultures were resuspended in 20 mM supplemented Tris-buffer and lysed by microfluidizer at 18 k PSI (M-110P Microfluidics, Inc.). The soluble fraction was passed through 3 ml of nickel nitrilotriacetic acid agarose (Ni-NTA) (Qiagen), washed with 20 mM imidazole, and eluted with 500 mM imidazole. Pure proteins with the correct homooligomeric conformation were collected from a Superoser™ 6 10/300 GL SEC column (GE Healthcare) in Tris-buffer (TBS; 25 mM Tris, 150 mM NaCl, 5% glycerol). Separately expressed components were kept at a concentration of ˜200 μM at 4° C.

SpyTag-spyCatcher™ conjugation was done by mixing a tagged protein and the complementary tagged array component at a 1.3:1 molar ratio, overnight incubation (˜10 hours) at 4° C. followed by Superose™ 6 10/300 GL SEC column purification to obtain only fully conjugated homooligomers. Sub-loaded conjugation was done at tag:array protein 0.17:1 molar ratio and used as is. Biotinylation of AV-tagged components was performed with BirA as described in [⁴²] and followed by Superose™ 6 10/300 GL SEC column purification. In-vitro array assembly was induced by mixing both array components at equimolar concentration.

GFP-tagged 60-mer nanocages were expressed and purified as previously.³⁰ GBP-mScarlet was expressed in E. coli BL21 Rosetta™ 2 (Stratagene) by induction with 1 mM IPTG in 2×YT medium at 20° C. overnight. Bacteria were lysed with a microfluidizer at 20 kPsi in lysis buffer (20 mM Hepes, 150 mM KCl, 1% TritonX100, 5% Glycerol, 5 mM MgCl₂, pH 7.6) enriched with protease inhibitors (Roche Mini) and 1 mg/ml lysozyme (Sigma) and 10 μg/ml DNAse I (Roche). After clarification (20000 rpm, Beckman J A 25.5, 30 min 4° C.), lysate was incubated with Glutathione S-sepharose 4B resin (GE Healthcare) for 2 h at 4C and washed extensively with (20 mM Hepes, 150 mM KCl, 5% glycerol, pH7.6), and eluted in (20 mM Hepes, 150 mM KCl, 5% glycerol, 10 mM reduced glutathione, pH7.6). Eluted protein was then cleaved by adding 1:50 (vol:vol) of 2 mg/mL (His)_{G}-TEV protease and 1 mM/0.5 mM final DTT/EDTA overnight at 4° C. The buffer of the cleaved protein was then exchanged for (20 mM Hepes, 150 mM KCl, 5% Glycerol, pH 7.6) using a ZebaSpin™ column (Pierce), and free GST was removed by incubation with Glutathione S-sepharose 4B resin. Tag-free GBP-mScarlet was then ultracentrifuged at 100,000×g for 5 min at 4C to remove aggregates. GBP-mScarlet was then incubated with GFP-60mer nanocages,³⁰ followed by size exclusion chromatography (see Microscope calibration), which further removed the TEV protease from the final mScarlet-GBP/GFP-60mer.

GBP-EGFR-Darpin was expressed similarly as GBP-mScarlet, except that lysis was performed using sonication, lysate clarification was performed at 16,000 rpm in a Beckman JA 25.5 rotor for 30 min at 4° C.). After TEV cleavage buffer was exchanged for (20 mM Hepes, 150 mM KCl, 5% Glycerol, pH 7.6) by dialysis, free GST and TEV proteases were removed by sequential incubation with Glutathione S-Sepharose™ 4B resin and Ni-NTA resin. Tag-free GBP-EGFR-Darpin was then flash frozen in liquid Ni and kept at −80° C.

Delta-like ligand 4 (DLL4) was prepared from a fragment of the human Delta ectodomain (1-405) with a C-terminal GS-SpyTag-6×His sequence. The protein was purified by immobilized metal affinity chromatography from culture medium from transiently transfected Expi293F cells (Thermo Fisher), then further purified to homogeneity by size exclusion chromatography on a Superdex™ 200 column in 50 mM Tris, pH 8.0, 150 mM NaCl, and 5% glycerol, and flash frozen before storage at −80° C. DLL4 was conjugated to the SpyCatcher tagged A homooligomers (ASC) at 1.5:1 molar ratio of DLL4 to ASC. The ASC-ST-DLL4 conjugate was purified by size exclusion chromatography on a Superose™ 6 column. The ASC-ST-DLL4-JF646 conjugate was produced by coupling of 1.5 μM ASC-ST-DLL4 to excess Janelia Fluor 646 SE (Tocris) overnight at 4° C. in 25 mM HEPES, pH 7.5, 150 mM NaCl. The labeled ASC-ST-DLL4 was then purified by desalting on a P-30 column (Bio-Rad). The final molar ratio of JF646 to ASC-ST-DLL4 was 5:1.

### Negative-Stain Electron Microscopy.

For initial screening of coexpressed designs insoluble fractions were centrifuged at 12,000 g for 15 min and resuspended in Tris-buffer (TBS; 25 mM Tris, 300 mM NaCl) twice prior to grid preparation. Samples were applied to glow-discharged EM grids with continuous carbon, after which grids were washed with distilled, deionized water, and stained with 2% uranyl formate. EM grids were screened using an FEI Morgagni 100 kV transmission electron microscope possessed of a Gatan Orius™ CCD camera. For the working design EM grids were initially screened using the Morgagni. Micrographs of well-stained EM grids were then obtained with an FEI Tecnair™ G2 Spirit transmission electron microscope (equipped with a LaB6 filament and Gatan UltraScan™ 4 k×4 k CCD camera) operating at 120 kV and magnified pixel size of 1.6 Å. Data collection was performed via the Leginon™ software package.⁵⁰ Single-particle style image processing (including CTF estimation, particle picking, particle extraction, and two-dimensional alignment and averaging) was accomplishing using the Relion™ software package.⁵¹

### Kinetic Optical Characterization of In Vitro Assemblies.

Arrays formation kinetics was determined by turbidity due to light scattering, monitored by absorption at 330 nm wavelength, using an Agilent Technologies (Santa Clara. Calif.) Cary 8454 UV-Vis spectrophotometer. Control sample containing a single component at 20 μM was measured for 3 hours. Kinetic measurements were initiated immediately after mixing both components in equimolar concentrations between 1 μM to 2 μM.

### Protein Stabilization Characterization.

Far-ultraviolet Circular Dichroism (CD) measurements were carried out with an AVIV spectrometer, model 420. Wavelength scans were measured from 260 to 195 nm at temperatures between 25 and 95° C. Temperature melts monitored absorption signal at 220 nm in steps of 2° C./min and 30 s of equilibration time. For wavelength scans and temperature melts a protein solution in PBS buffer (pH 7.4) of concentration 0.2-0.4 mg/ml was used in a 1 mm path-length cuvette.

### SAXS Experiments.

Small angle X-ray scattering data were collected at the SIBYLS beamline at the Advanced Light Source in Berkeley California.⁵² Components A and B were measured independently and as a mixture in 25 Tris, 150 NaCl and 5% glycerol. Imidazole was added to the mixture in a stepwise fashion after A and B were mixed 1:1. These solutions were prepared 24 hours prior to collection. Before collection samples were placed in a 96 well plate. Each sample was presented to the X-ray beam using an automated robotics platform. The 10.2 keV monochromatic X-rays at a flux of 10¹² photons per second struck the sample with a 1×0.3 mm rectangular profile that converged at the detector to a 100 μm×100 μm spot. The detector to sample distance was 2 m and nearly centered on the detector. Each sample was exposed for a total of 10 seconds. The Pilatus 2M detector framed the 10 second exposure in 300 ms frames for a total of 33 frames. No radiation damage was observed during exposures.

Components A and B were independently collected at 4 concentrations (40, 80, 120, 160 μM). No concentration dependence was observed so the 160 μM, highest signal, SAXS measurement was fully analyzed using the Scatter program developed by Rambo et al at SIBYLS and the Diamond Light Source. SAXS profiles were calculated using the FOXS⁵³ and compared to the measured data with excellent agreement χ²<1 for hexameric A and tetrameric B(FIG. 14a). No further processing was conducted as the agreement between calculated SAXS from the model and the experiment was sufficient to verify close agreement of the atomic model.

The mixture of components A and B were measured at 4 concentrations as well (0.5, 2, 5, and 10 μM). The scattering profiles all had peaks (FIG. S2.e and FIG. S9.a, d, f) at q spacings. The scattering can be described in several ways according to scattering theory. In crystalline systems the diffraction intensity is the convolution of the lattice and the asymmetric unit within the lattice.⁵⁴ Below we will distinguish the peaks as a diffraction component and the asymmetric unit as the scattering component. A very good match of Bragg spacings with the diffraction observed comes from calculating a P6 lattice with a 303 Å spacing. The calculation was done using a CCP4 script based on the “unique” command which generates a unique set of reflection given a symmetry and distances.⁵⁵

The measured SAXS profile was also matched by calculations of the SAXS from atomic models (FIGS. 2.e and 14c). Atomic model sheets were created by increasing the number of Asymmetric Units (ASUs) defined as 12 monomers: 6 belonging to the A Hexamer and 6 to 3 halves of the surrounding B tetramers (see FIG. 14a rightmost panel). Array counting 10, 13, 17, 21, 26, 31, 37, 75, 113, and 188 ASUs along the P6 lattice were used for SAXS profiles modeling using FOXS. The calculated SAXS profiles have diffraction peaks placed in agreement with the measured data. As per scattering theory⁵⁶ the diffracting from the lattice increased relative to the scattering from the asymmetric unit as the sheet size increased. The diffraction to scattering ratio in the measured profiles are larger than those in all calculated profiles indicating that the sheets are larger in solution than the largest models we created.

We utilized the trend in the ratio of the diffraction to scattering from the models to estimate the size of the sheets observed in solution. All calculations and the experimental SAXS profiles were scaled by the underlying scattering. The higher the angle, the smaller the contribution of the diffraction, so the highest angle experimental signal with sufficient signal to noise was used (0.1<q<0.15 Å) for this scaling all profiles relative to one another. Once scaled, the ASU was divided through all scattering curves where the ASU is as defined above. By dividing through, the exponential decay of the scattering profile was removed and yielded a set of peaks that oscillate about a constant background which was further normalized so as to oscillate about a value of one (FIG. 2.c and S9.d) over a useful q range between 0.01<q<0.1 Å⁻¹. The intensity difference between the first minimum and first maximum peak from all calculated profiles was tabulated and the trend was fit to the number of ASUs (x) using two simple formulas: 1) exponential form: k1*exp^{k2*x}+k3 [k1=−0.2.2, k2=3.5, k3=−1.6], 2) polynomial form: k1*x^{k2}+k3 [k1=64.5, k2=4.3, k3=8.9]. A reasonable fit was obtained for the exponential form as shown in FIG. 14c. Extrapolating from this fit, the average array consists of 6000 ASUs (2000 using the polynomial fit) and assuming a circular array shape it average size would be 1.8 μm in diameter (1.05 using the polynomial fit).

Time resolved SAXS measurements were obtained for mixtures at 10 μM at several time points ranging from 30 see to 15 min. Each measurement was collected from a separate well to avoid accumulated damage to the samples. SAXS profiles were scaled (including the ovemight SAXS profile to which a fit was obtained) and the ASU was divided. The min to max peaks distance was calculated and scaled for all profiles to agree with the values obtained for the common sample (the overnight sample the fit was obtained for in FIG. 14e). The exponential fit above was then applied to estimate the transient dimensions at each time point obtained by the SEXS measurement (FIG. 1f, g).

### Cell Culture:

Flp-In NIH/3T3 cells (Invitrogen, R76107) were cultured in DMEM (Gibco, 31966021) supplemented with 10% Donor Bovine Serum (Gibco, 16030074) and Pen/Strep 100 units/ml at 37° C. with 5% CO₂. Cells were transfected with Lipofectamine 2000 (Invitrogen, 11668). Stable transfectants obtained according to the manufacturer's instructions by homologous recombination at the FRT were selected using 100 Wpg/mL Hygromycin B Gold™ (Invivogen, 31282-04-9). HeLa cells were cultured in DMEM supplemented with 10% Fetal Bovine Serum and Penicillin-streptomycin 100 units/ml at 37° C. with 5% CO₂.

Human Umbilical Vein Endothelial Cells (HUVECs) (Lonza, Germany) were grown on 0.1% gelatin-coated 35 mm cell culture dish in EGM2 media (20% Fetal Bovine Serum, 1% penicillin-streptomycin, 1% Glutamax (Gibco, catalog #35050061), 1% ECGS (endothelial cell growth factors), 1 mM sodium pyruvate, 7.5 mM HEPES, 0.08 mg/ML heparin, 0.01% amphotericin B, a mixture of 1×RPMI 1640 with and without glucose to reach 5.6 mM glucose in final volume). HUVECs were expanded till passage 4 and cryopreserved.

ECGS was extracted from 25 mature whole bovine pituitary glands from Pei-Freeze biologicals (catalog #57133-2). Pituitary glands were homogenized with 187.5 mL ice cold 150 mM NaCl and the pH adjusted to pH4.5 with HCl. The solution was stirred in a cold room for 2 hours and centrifuged at 4000 RPM at 4C for 1 hour. The supernatant was collected and adjusted to pH7.6, 0.5 g/100 mL streptomycin sulfate (Sigma #S9137) was added, stirred in the cold room overnight and centrifuged 4000 RPM at 4C for 1 hour. The supernatant was filtered using a 0.45 to 0.2-micrometer filter.

The HUVEC cells were expanded till P8, followed by 16 hrs starvation with DMEM low glucose media prior to protein scaffold treatment. The cells were then treated with desired concentrations of protein scaffolds in DMEM low glucose media for 30 min or 60 min. Cells were cultured at 37C, 5% CO₂, 20% O₂.

### Fluorescent Microscopy of In Vivo Assemblies in Bacteria.

Glycerol stocks of E. coli strain BL21(DE3) having the single cistronic AGFP and the bicistronic AGFP+B were used to grow overnight cultures in LB medium+KAN at 37° C. To avoid GFP signal saturation, leaky expression only was used by allowing culture to remain at 37° C. another 24 hours before spotted onto a 1% agarose-LB-KAN pad. Agarose pads were imaged using the Leica SP8X confocal system to obtain bright and dark field images.

### Characterization of Array-Induced Protein Relocalization and Array Growth Dynamics on Cells

All live imaging of NIH-3T3 cells (FIGS. 4a-d, 5a-j,l-m, S11, S16, S18) was performed in Leibovitz's L-15 medium (Gibco, 11415064) supplemented with 10% Donor Bovine Serum and HEPES (Gibco, 1563080, 20 mM) using the custom spinning disk setup described below. For protein relocalisation by preformed arrays experiments, GBP-TM-mScarlet expressing NIH/3T3 cells were spread on glass-bottom dishes (World Precision Instruments, FD3510) coated with fibronectin (Sigma, F1141, 50 g/ml in PBS), for 1 hour at 37° C. then incubated with 10 μl/mL of preformed arrays. Cells were either imaged immediately (FIG. 4 B,C) or incubated with the arrays for 30 minutes (FIG. 4). Preformed arrays were obtained by mixing equimolar amounts (1 μM) of AGFP mixed with B in the presence of 0.5M Imidazole overnight at RT in a 180 μl total volume. This solution was then centrifuged at 250,000×g for 30 minutes at 4° C. and resuspended in 50 μl PBS. For assembly on the surface of cells (FIG. 5), spread cells were incubated with B(C)GFP (1 μM in PBS) for 1 minute, rinsed in PBS, and imaged in serum/HEPES-supplemented L-15 medium. A was then added (0.2 μM in serum/HEPES-supplemented L-15 medium) during image acquisition.

### In Situ AFM Characterization.

Array growth and dynamics at molecular resolution were characterized by mixing both components at equimolar concentration (7 μM) and immediately injecting the solution into the fluid cell on freshly cleaved mica. All in-situ AFM images were collected using silicon probes (HYDRA6V-100NG, k=0.292 N m−1, AppNano™) in ScanAsyst Mode with a Nanoscope™ 8 (Bruker). To minimize damage to the structural integrity of the arrays during AFM imaging, the applied force was minimized by limiting the Peak Force™ Setpoint to 120 pN or less.³⁴ The loading force can be roughly calculated from the cantilever spring constant, deflection sensitivity and Peak Force Setpoint.

### Correlative SIM/AFM Characterization on Supported Bilayers

Arrays were assembled on supported bilayers (FIG. 5.k and also FIG. 22) in a manner mimicking assembly on cells (see above and also FIG. 5.a). Supported bilayers were formed according to the method of Chiaruttini and colleagues.⁵⁸ Briefly, a lipid mixture (1 mg/ml lipids in chloroform, 47.5% POPC, 47.5% DOPE, 5% DSPE-PEG(2000)-Biotin, 0.2% Rhodamine-PE, all from Avanti Polar Lipids) was used to form GUVs in [5 mM Hepes 300 mM Sucrose pH 7.5] in a Nanion Vesicle Prep Pro™. GUVs were then diluted 1:1 (vol:vol) in 20 mM Hepes 150 mM KCl pH 7.5. A clean-room grade coverslip (Nexterion, Schott, #1.5, 25×75 mm) was surface-activated under pure oxygen in a plasma cleaner (PlasmaPrep2, GaLa instruments) then assembled into a peelable flow chamber using a top 22×22 mm standard glass coverslip and a custom Silicon insert (SuperClear™ Silicone Sheet 40° shore A, 0.5 mm thickness, Silex Silicon, 25×75 mm insert with a 12×35 mm hole precisely cut with a Graphtec CE6000 cutting plotter). GUVs were burst onto the activated glass surface, and, after extensive washing with [20 mM Hepes, 150 mM KCl, pH 7.6], the glass surface was quenched with PLL-PEG (SuSoS, 1 mg/ml in 10 mM Hepes, pH 7.6) for 5 minutes, before further washing with [20 mM Hepes, 150 mM KCl, pH 7.6]. A solution of B(c)mSA2 (200 nM in 20 mM Hepes, 150 mM KCl, pH 7.6) was then flowed in and incubated for 1 min before extensive washes in (20 mM Hepes, 150 mM KCl, pH 7.6). Then, a solution of A(d)GFP (20 nM in 20 mM Hepes, 150 mM KCl, 500 mM Imidazole, pH 7.6) was flowed in and incubated for 5 min. Flow cell was then washed extensively with [20 mM Hepes, 150 mM KCl, pH 7.6], and sample fixed with 0.25% glutaraldehyde (weight:vol, EMS) in PBS for 5 min and 4% Paraformaldehyde (weight:vol, EMS) in PBS for 5 min. Fixatives were then removed by extensive washing in [20 mM Hepes, 150 mM KCl, pH 7.6]. The top 22×22 mm coverslip was then carefully removed, leaving the insert in place in order to hold a volume of imaging buffer (20 mM Hepes, 150 mM KCl, pH 7.6). This allowed simultaneous super-resolution Structured Illumination Microscopy (SIM) imaging through the bottom coverlip, and AFM imaging from the top of the open chamber (FIG. 23).

Correlative AFM/SIM imaging was performed by combining a Bioscope Resolve™ system (Bruker, Santa Barbara, Calif., LISA) with a home-made SIM system.⁵⁹ The fields of view of the two microscopes were aligned so that the AFM probe was positioned in the middle of the field of view of the SIM microscope. A brightfield image of the “shadow” of the AFM cantilever was used to precisely align the AFM probe with the SIM lens. To acquire structured illumination microscopy images, a ×60/1.2 NA water immersion lens (UPLSAPO 60×W, Olympus) focused the structured illumination pattern onto the sample, and the same lens was also used to capture the fluorescence emission light before imaging onto an sCMOS camera (C11440, Hamamatsu). The wavelengths used for excitation were 488 nm (iBEAM-SMART™-488, Toptica) for the protein arrays and 561 nm (OBIS 561, Coherent) for the lipid bilayers. Images were acquired using custom SIM software described previously.⁵⁹

AFM images were acquired in Fast Tapping imaging mode using Fastscan™-D probes (Bruker), with a nominal spring constant of 0.25 N/m and a resonant frequency of 110 kHz. Images were recorded at scan speeds ranging between 2 and 10 Hz and tip-sample interaction forces between 100 and 200 pN. Large scale images (20×20 μm) were used to register the AFM with the SIM fields of view and small (500×500 nm) scans were performed in order to resolve the structure of the arrays. Raw AFM images were first order fitted with reference to the lipid bilayer. Amplitude images were inverted and a lowpass filter was applied to remove excess noise. For the high magnification scans, amplitude images are presented as movement of the arrays on the lipid bilayer does not affect the resolution of these images to the same extent as that of topography images. Amplitude data is helpful in visualising features and the shape of the sample, however note that the z-scale in amplitude images indicates the amplitude error and thus is not representative of the height of the sample.

### Protein Extraction and Western Blot Analysis

Cells were lysed directly on the plate with lysis buffer containing 20 mM Tris-HCl pH 7.5, 150 mM NaCl, 15% Glycerol, 1% Triton x-100, 1 M β-Glycerolphosphate, 0.5 M NaF, 0.1 M Sodium Pyrophosphate, Orthovanadate, PMSF and 2% SDS. 25 U of Benzonase® Nuclease (EMD Chemicals, Gibbstown, N.J.), and 100× phosphatase inhibitor cocktail 2, 4× Laemli sample buffer (900 μl of sample buffer and 100 μl β-Mercaptoethanol) is added to the lysate then heated (95° C., 5 mins). 30 μl of protein sample was run on SDS-PAGE (protean TGX pre-casted gradient gel, 4%-20%, Bio-rad) and transferred to the Nitro-Cellulose membrane (Bio-Rad) by semi-dry transfer (Bio-Rad). Membranes are blocked for 3 h with 5% BSA (P-AKT) or 1 h with 5% milk (β-Actin) corresponding to the primary antibodies and incubated in the primary antibodies overnight at 4° C. The antibodies used for western blot were P-AKT(S473)(Cell Signaling 9271, 1:2000), β-Actin (Cell Signaling 13E5, 1:1000). The membrane incubated with P-AKT was then blocked with 5% milk prior to secondary antibody incubation. The membranes were then incubated with secondary antibodies anti-rabbit IgG HRP conjugate (Bio-Rad) for 2 hrs and detected using the Immobilon-luminol reagent assay (EMP Millipore).

### Cell(Immuno)Staining

For FIG. 4e-f and FIG. 18, cells were fixed in 4% paraformaldehyde in PBS for 15 min, washed with PBS (3×5 mins) and blocked for 1 h in 3% BSA (Fisher bioreagents CAS 9(48-46-8) and 0.1% Triton X-100 (Sigma 9002-93-1). The cells were then incubated in primary antibody overnight, washed with PBS (3×5 min), incubated with the secondary antibody in 3% BSA and 0.1% Triton X-100 for 1 hr, washed (4×10 mins, adding 1 μg/ml DAPI in 2nd wash), mounted (Vectashield™, VectorLabs H1400) and stored at 4° C. The antibodies for immunostaining were anti-Tie2 (Cell Signaling AB33, 1:100); CD31 (HD Biosciences 555444, 1:250); VE-cadherin (BD Biosciences 555661, 1:250); Alexa 647-conjugated secondary antibody (Molecular Probes) and Phalloidin conjugated with Alexa Fluor 568 (invitrogen A12380, 1:100).

Alternatively, for FIG. 5l,m and S17f, HeLa cells spread on fibronectin-coated glass bottom dishes and treated with A/B were fixed in 4% paraformaldehyde in PBS for 20 min, permeabilized with 0.05% saponin (Sigma) in PBS for 5 min, then washed in PBS, then in PBS-1% BSA for 5 min, then in PBS. Cells were then incubated with anti LAMP1 antibodies (Developmental Studies Hybridoma Bank, clone H4A3 1:500) in PBS-1% BSA for 20 min, then washed thrice in PBS, then incubated with anti-mouse F(ab′)2-Alexa647 (Invitrogen) secondary antibodies at 1:500 in PBS-1% BSA for 20 min. Cells were then washed thrice in PBS. Imaging was performed in PBS instead of mounting medium to avoid squashing the cells, thereby biasing the array/lysosome colocalization.

Alternatively, to label cell membranes of fixed NIH/3T3 cells expressing GBP-TM-mScarlet (FIG. 10n,o) Alexa 633-wheat germ agglutinin (Thermo Fisher, 1:1000 in PBS for 1 min). Fixation and imaging in PBS was performed as above.

### Endocytic Block

To evaluate the endocytic block affecting clustered EGF receptors (FIG. 5.l,m), HeLa cells were plated on glass-bottom dishes (World Precision Instruments, FD3510) coated with fibronectin (Sigma, F1141, 50 μg/ml in PBS), for 2 hour at 37° C. DMEM-10% serum, then serum-starved overnight in DMEM-0.1% serum. Cell were then incubated with 20 ug/mL GBP-EGFR-Darpin in DMEM-0.1% serum for Imin at 37° C., then washed in DMEM-0.1% serum, then incubated with 0.5 μM B(c)GFP in DMEM-0.1% serum for Imin at 37° C., then washed in DMEM-0.1% serum, then 0.5 μM A in DMEM-0.1% serum was added (or not) for 1 min at 37° C. Cells were then chased for a varying amount of time in DMEM-0.1% serum at 37° C. before fixation, immunofluorescence against LAMP1 (see above), and spinning disk confocal imaging followed by unbiased automated image quantification (see below).

Alternatively, for FIG. 24a,b, cells were treated with GBP-EGFR-Darpin as above, then 100 μM of GFP-60mer nanocages was added in DMEM-0.1% serum for 1 min at 37° C. prior to chasing in DMEM-0.1% serum at 37° C., fixation, LAMP1 immunofluorescence and imaging/quantification. Control in this case was the unassembled trimeric building block of the GFP-60mer.

To quantitatively measure the internalization of GFP-positive arrays as a function of their size (FIG. 5. m,o), we could not use the colocalization with LAMP1 as above, as the GBP-TM-mScarlet construct is not routed to lysosomes upon endocytosis (presumably routed to recycling endosomes). We thus relied on a membrane marker and quantified the amount of signal at the plasma membrane versus inside the cell. Experimentally, stable NIH/3T3 cells expressing GBP-TM-mScarlet under Doxycycline (Dox)-inducible promoter were treated with varying doses of Doxycycline for 24 h, then cells were spread on fibronectin-coated coverslips for 1 h as above, then incubated with 0.5 μM B(e)GFP in serum-supplemented DMEM medium for 1 min at 37° C., rinsed in PBS, then 0.5 μM unlabelled A was added (or not) serum-supplemented DMEM medium for 1 min at 37° C. After a 60 min chase in serum-supplemented DMEM medium at 37° C., cells were briefly incubated with Alexa-633-coupled Wheat Germ Agglutinin to label cell membranes, then cells were fixed, imaged by spinning disk confocal microscopy and images were processed for automated image analysis (see below).

### Flow Cytometry

To measure the density of active GBP-TM-mScarlet at the surface of cells as a function of the expression level of this construct (FIG. 23k), stable NIH/3T3 cells expressing GBP-TM-mScarlet under Doxycycline-inducible promoter were treated with varying doses of Doxycycline for 24 h, then cells were incubated with 1 μM purified GFP in scrum/HEPES-supplemented L-15 medium for Imin at RT, then wash in PBS-1 mM EDTA, then trypsinized and resuspended in serum/HEPES-supplemented L-13 medium GFP-fluorescence per cell was then measured by Flow cytometry in an iCyt Eclipse™ instrument (Sony) using a 488 am laser. Data analysis was performed using the supplier's software package.

### Imaging

TIRF imaging of array assembled onto cells (FIG. 5f,g) was performed on a custom-built TIRF system based on a Nikon Ti stand equipped with perfect focus system, a fast Z piezo stage (ASI), an azimuthal TIRF illuminators (iLas2™, Roper France) modified to have an extended field of view (Cairn) and a PLAN™ Apo 1.45 NA 100× objective. Images were recorded with a Photometrics Prime™ 95B back-illuminated sCMOS camera run in pseudo global shutter mode and synchronized with the azimuthal illumination. GFP was excited by a 488 nm laser (Coherent OBIS mounted in a Cairn laser launch) and imaged using a Chroma 525/50 bandpass filter mounted on a Cairn Optospin™ wheel. System was operated by Metamorph™. This microscope was calibrated to convert fluorescence intensity into approximate molecule numbers (see calibration chapter above and FIG. 23).

For fast imaging of array formation (FIG. 5,21, 23, 24), receptor recruitment by preformed arrays (FIGS. 4b-d and 16), quantitative imaging of the endocytic block effect (FIG. 5, 24), calibrated molecular ratios (FIGS. 5 and 23), and Fluorescence Recovery After Photobleaching (FRAP; FIG. 16), imaging was performed onto a custom spinning disk confocal instrument composed of Nikon Ti stand equipped with perfect focus system, a fast Z piezo stage (ASI) and a PLAN™ Apo Lambda 1.45 NA 100× (or PLAN™ Apo Lambda 1.4 60×) objective, and a spinning disk head (Yokogawa CSUX1). Images were recorded with a Photometrics Primer™ 95B back-illuminated sCMOS camera run in pseudo global shutter mode and synchronized with the spinning disk wheel. Excitation was provided by 488, 561 or 630 nm lasers (all Coherent OBIS mounted in a Cairn laser launch) and imaged using dedicated single bandpass filters for each channel mounted on a Cairn Optospin™ wheel (Chroma 525/50 for GFP and Chroma 595/50 for mCherry/mScarlet and Chroma ET6551p for WGA-637 and Alexa 647). FRAP was performed using an iLAS2™ galvanometer module (Roper France) mounted on the back port of the stand and combined with the side spinning disk illumination path using a broadband polarizing beamsplitter mounted in a 3D-printed fluorescence filter cube. To enable fast 4D acquisitions, an FPGA module (National Instrument sbRIO-9637 running custom code) was used for hardware-based synchronization of the instrument, in particular to ensure that the piezo z stage moved only during the readout period of the sCMOS camera. Temperature was kept at 37° C. using a temperature control chamber (MicroscopeHeaters.Com, Brighton UK). System was operated by Metamorph™. This microscope was calibrated to convert fluorescence intensity into approximate molecule numbers (see FIG. 23).

Imaging of immunofluorescence experiments depicted in FIG. 4e-f, on GE DeltaVision™ OMX SR super-resolution microscope using 60× objective and OMX software and Imaris software. The images in FIG. 18 were taken in Nikon A1R confocal microscope using 60× objective.

### Microscope Calibration and Comparison Between Preformed Arrays and Arrays Made on Cells

To calibrate the TIRE and Spinning disk setup described above in terms of estimated number of GFP and mScarlet molecules, we mixed our previously published GFP-60mer nanocages³⁰ with an excess of a purified GBP-mscarlet fusion (see FIG. 23c). Excess of unbound GBP-mscarlet was then removed by size exclusion chromatography on a superose 6 column, and GBP-mScarlet induced a shift in molecular weight of the GFP-60mer (see FIG. 23d). Near 1:1 binding ratio was confirmed by absorbance measurement at 490 nm and 561 nm. Indeed, absorbance of mScarlet-GBP/GFP-60mer at 570 nm was 0.091 so 907 nM of mScarlet (Extinction coefficient of mscarlet is 100,330 and it does not absorb at 470 nm). On the other hand, absorbance at 490 nm was 0.092, so 862 nM of GFP after correction for mScarlet absorbance at 490 nm. This gives a ratio GFP/mScarlet of 0.95. We found that the GFP fluorescence of the mScarlet-GBP/GFP-60mer nanocages was almost identical to that of GFP-60mer nanocages, suggesting that FRET with mScarlet does not lower the fluorescence of GFP (or that it is compensated by the increase of fluorescence due to the “enhancer” nanobody we used (38)).

We then acquired z-stacks of diluted nanocages in the same buffer as the cells' imaging medium, which revealed discrete particles fluorescing on both the GFP and mScarlet channels (see FIG. 23e). We then z-projected the planes containing particle signal (maximum intensity projection), and automatically detected the particles by 2D Gaussian fitting using the Thunderstorm algorithm⁶⁰. We then assessed the colocalization between GFP and mScarlet-positive particles by considering colocalized particles whose distance between GFP and mScarlet fluorescence centroid is below 200 nm. Non colocalizing particles were discarded, and we then estimated the average fluorescence of one 60-mer by computing the median of the integrated fluorescence intensity from the gaussian fitting (minus the background) for each channel (FIG. 23e-f). By dividing this median fluorescence by the number of GFP/mCherry per nanocage (i.e. 60), we can estimate the fluorescence of one GFP (respectively one mScarlet) molecule. From this, we can evaluate the approximate number of GFP and mScarlet molecules per diffraction-limited spot on a cell by keeping the exposure and laser power constant between calibration and experiment (see equations below for derivation of the estimated error estimated on these measurements).

FIG. 23f shows that fluorescence intensity increases linearly with exposure time, suggesting that the instrument (spinning disk in this case) operates in its linear range. This calibration was done for each microscope and to ensure that laser fluctuations were not a variable, calibration datasets were acquired on the same day as an experiment. Care was taken to perform these measurements in areas of the field of view where illumination was homogenous (about 50% for the spinning disk and about 80% for the TIRF). Note that because of azimuthal illumination, our TIRF instrument does not suffer from shadowing effects, and that for FIG. 5h, we used 60mer-GFP (not mScarlet-GBP/GFP-60mer) calibration nanocages.

Mathematically, conversions into number of molecules, and their associated error, were performed by building on the elegant work of Picco and colleagues as follows:⁶¹ I_{GFP} is the integrated intensity of the arrays in the GFP channel (n measurements) and I_{60GFP} is the integrated intensity of the reference 60mer in the same channel (n′ measurements). As distribution of dim signals are skewed, estimated average values for I_{GFP}, noted [symbol] , is computed as median of the distribution. The estimate for the reference 60 mer, [symbol] , is similarly computed from I_{60GFP}. The respective error associated with these measurements, [symbol] and [symbol] , respectively, are estimated with the Median Absolute Deviation (MAD) corrected for asymptotically normal consistency on the natural logarithm transform of the raw fluorescence values I_{GFP} and I_{60GFP}.

$$= exp ( median ( ln ( {I}_{GFP} ) ) ) = median ( {I}_{GFP} )$$

$$= \times \frac{1.4826 \times MAD ( ln ( {I}_{GFP} ) )}{\sqrt{n}}$$

$$= median ( {I}_{60 GFP} )$$

$$= \times \frac{1.4826 \times MAD ( ln ( {I}_{60 GFP} ) )}{\sqrt{{n}^{′}}}$$

The estimate of number of GFP molecule per army was computed a

$$= \frac{}{} \times 60$$

The uncertainty over this number of molecules, δ_{n GFP}, was computed by error propagation as

$$= \sqrt{( \frac{60}{} \times ) + {( 60 \times \frac{{( )}^{2}}{} )}^{2}}$$

Similarly, the number of molecules in the mScarlet channel, n_{mScarlet} was estimated from I_{mScarlet}, the integrated intensity of the arrays in the mScarlet channel (n measurements) and the intensity of the reference 60mer in the same channel, I_{60mScarlet} (n′ measurements).

$$= exp ( median ( ln ( {I}_{mScarlet} ) ) ) = median ( {I}_{mScarlet} )$$

$$= \times \frac{1.4826 \times MAD ( lnln ( {I}_{mScarlet} ) )}{\sqrt{n}}$$

$$= median ( {I}_{60 mScarlet} )$$

$$= \times \frac{1.4826 \times MAD ( ln ( {I}_{60 mScarlet} ) )}{\sqrt{{n}^{′}}}$$

The estimate of number of mScarlet molecules per array was computed as

$$= \frac{}{} \times 60$$

The uncertainty over this number of molecules, δ_{n GFP}, was computed by error propagation as

$$= \sqrt{{( \frac{60}{} \times )}^{2} + {( 60 \times \frac{{( )}^{2}}{} )}^{2}}$$

We then estimated the GFP/mscarlet ratio on cells in terms of molecules,

$$\frac{}{} \qquad ( Fig \text{.5} . g ) .$$

Its associated error,

$$δ \frac{m Scarlet}{}$$

is computed as:

$$δ \frac{m Scarlet}{} = \sqrt{( \frac{}{} ) + {( \frac{}{} )}^{2}}$$

To compare the lattice order between arrays made on cells and preformed arrays (FIG. 5.k), we formed B(c)GFP/A(d)mScarlet arrays on cells or in vitro, then imaged them and measured array fluorescence by gaussian fitting as above. Preformed arrays were obtained by mixing 5 μM B(c)GFP with 5 μM A(d)mScarlet in (TBS-0.5M Imidazole) for 4 h at RT, followed by ultracentrifugation (250,000×g 30 min) and dilution into PBS for imaging onto the same dishes as the cells. We verified the order of these arrays by EM (FIG. 20d). Using the notations introduced above, we measured the mScarlet/GFP ratio as

$$GFP = \frac{}{}$$

Its associated error, [symbol] is computed as:

$$= \sqrt{{( )}^{2} + {( )}^{2}}$$

We verified that the mScarlet/GFP fluorescence ratio varies as expected from the structure, and is thus a good proxy of bulk order (FIG. 23i). To do so, we formed B(c)GFP/A(d)mScarlet arrays in vitro as above, then incubated them with a 2-fold molar excess of GBP-mScarlet over B(c)GFP for 1h at RT, followed by ultracentrifugation (250,000×g 30 min) and dilution into PBS for imaging onto the same dishes as the cells. As binding of the GBP-mScarlet to GFP does not effectively modify the fluorescence of GFP (see above), the predicted variation of the mScarlet/GFP fluorescence ratio upon saturation of each GFP by GBP-mScarlet is:

( [symbol] GFP)_{with GFP-mScarlet}=3/2×( [symbol] /GFP)_{without GBP-mScarlet}

To estimate the A/B ratio on cells (FIG. 23j) we incubated cells with B(c)GFP and A(d)mScarlet. As the distance between GFP and mScarlet within the arrays is =6.09 nm, there is significant FRET between the two molecules. The FRET efficiency is given by

$$E = \frac{1}{1 + {( r / {R}_{0} )}^{6}} = 0.39$$

with R₀=56.75. To the GFP intensity l_{GFP} is corrected by a factor

$$\frac{1}{1 - E}$$

to account for FRET in order to evaluate [symbol] as above.

As dihedral components have twice more fluorophore than cyclic ones per unit cell, the mean A/B ratio, noted [symbol] computed as follows:

$$=$$

Its associated error, [symbol] is computed as:

$$= \sqrt{{( )}^{2} + {( )}^{2}}$$

### Statistics

Unless stated otherwise, measurements are given in mean±SEM. No randomization methods were used in this study. No blind experiments were conducted in this study. Statistical analyses were performed using GraphPad Prism 8 or SigmaStat 3.5 with an alpha of 0.05. Normality of variables was verified with Kolmogorov-Smirnov tests. Homoscedasticity of variables was always verified when conducting parametric tests. Post-hoc tests are indicated in their respective figure legends.

### Image Processing

Unless stated otherwise, images were processed using Fiji⁶²/ImageJ 1.52 d, Imaris, OMERO⁶³ and MATLAB 2017b (Mathworks) using custom codes available on request. Figures were assembled in Adobe Illustrator 2019 and movies were edited using Adobe Premiere pro CS6.

Spatial drift during acquisition was corrected using a custom GPU-accelerated registration code based on cross correlation between successive frames. Drift was measured on one channel and applied to all the channels in multichannel acquisitions.

For live quantification of mScarlet recruitment by preformed AGFP+B arrays (FIG. 4c), the array signal was segmented using a user-entered intensity threshold (bleaching is minimal so the same threshold was kept throughout the movie) and the mean mScarlet intensity was measured within this segmented region over time after homogenous background subtraction. The local mScarlet enrichment is then computed as the ratio between this value and the mean mScarlet intensity after background subtraction of a region of the same size but not overlapping with the array.

For 3D reconstruction (FIG. 4d and FIG. 15b), confocal z-stack of cells (Δz=200 nm) were acquired, and cell surface was automatically segmented in 3D using the Fiji plugin LimeSeg™ developed by Machado and colleagues.⁶⁴ 3D rendering was performed using Amira software.

For analysis of FRAP data of GBP-TM-mScarlet clustered by preformed AGFP+B arrays (FIG. 16c-d), since the GFP signal was used to set the area to bleach for mScarlet, we segmented the GFP signal using an intensity threshold and measured the intensity of the mScarlet signal in this region over the course of the experiment (pre-bleach and post bleach). This is justified as our FRAP setup only bleaches mScarlet (and not GFP), and the photobleaching of GFP due to imaging is limited (about 20% during the time course of the acquisition, see FIG. 16). Background was then homogeneously subtracted using a ROI outside the array as a reference, and intensity was then normalized using the formula:

$${I}_{norm} ( t ) = \frac{I ( t )}{{I}_{prebleach}}$$

with I(t), the mean intensity at time point t; I_{prebleach} the intensity before bleaching (averaged over six time points). As a control that binding of AGFP alone (that is, not in an array) does not affect fluorescence recovery of GBP-TM-mScarlet (meaning that the array does not recover because all the GBP-TM-mScarlet is trapped by the AGFP+B array), we performed FRAP experiments of GBP-TM-mScarlet in cells incubated with AGFP alone. As expected, we found that it recovers (FIG. 16d).

For live quantification of array assembly and growth on cells (FIG. 5.c-d, FIG. 23b, FIG. 24c), BGFP and mScarlet foci were first automatically detected in each frame by 2D Gaussian fitting using the Fiji Plugin Thunderstorm⁶⁰. Then, to objectively address the colocalization between BGFP and mScarlet foci, we used an object based method⁶⁵, where two foci are considered colocalised if the distance between their fluorescent centroids is below 200 nm, which is close to the lateral resolution of the microscope. To measure the GFP and mScarlet fluorescence of colocalising foci over time (FIG. 5.d) the trajectories of BGFP foci were first tracked using the MATLAB adaptation by Daniel Blair and Eric Dufresne of the IDL particle tracking code originally developed by David Grier, John Crocker, and Eric Weeks (Web site; physics.georgetown.edu/madab/index.html). Tracks were then filtered to keep only GFP-tracks that were found to colocalize with a mScarlet foci (that is if distance between GFP and a mScarlet fluorescence centroids is below 200 nm) and that had at least 150 timepoints. Foci intensity was then measured by measuring the maximum intensity in a 4-pixel diameter circle centred on the fluorescence centroid after background subtraction. Then, for each time point, the fluorescence of all the BGFP foci present in this time point, and their corresponding mScarlet foci, was averaged (FIG. 5.c). To evaluate the array nucleation rate, we downsampled our dataset into a series of small regions of interest of equal size (35 μm) in regions of the cells where the membrane was in focus (>14 regions per concentration of A). We then tracked all BGFP foci as above in each region. We then averaged the number of tracks present per region over time (FIG. 23b left panel). The intensity over time of each array was then measured as above and averaged across all arrays and all FOVs (FIG. 23b middle panel). The average initial velocity was then measured on these curves to generate the right panel of FIG. 23.b.

For Mean Square Displacement (MSD) analysis (FIG. 20a), the MSD of segments of increasing duration (delay time t) was computed (MSD(t)=<(Δx)²>+<(Δy)²>) for each GFP-positive track using the MATLAB class MSD Analyzer⁵⁸ (n=2195 tracks in N=3 cells). We then fitted the first 30 points weighted mean MSD as a function of delay time to a simple diffusion model captured by the function MSD (t)=4D, _{eff}tωιτηD _{eff}τηε effective diffusion rate (R²=0.9999; D_{eff}=0.0005 μm²/s).

For automated quantification of the colocalization between GFP-positive arrays and LAMP1 staining (FIG. 5.m), the raw data consisted of 3D confocal stacks (Δz=200 nm) of cells in both channels (GFP/LAMP1). We first automatically segmented the GFP channel by 2D gaussian fitting using Thunderstorm™⁶⁰ as above for each z-plane. To automatically segment the LAMP1 channel, we could not use 2D gaussian fitting, as the signal is not diffraction limited, so instead we relied on unbiased intensity thresholding set at the mean plus two standard deviations of the signal's intensity distribution in the brightest z-plane after homogenous background subtraction. This intensity threshold was kept constant across all z-planes of the same cell, but could vary between cells depending on the strength of the staining in each cell. We then scored each GFP-positive spot as colocalised if its fluorescence centroid was contained within a LAMP1-positive segmented region. The percentage of colocalization is then computed as:

$$\% of colocalization = \frac{\sum colocalizing particles}{\sum total particles} \times 100$$

This measurement was then averaged for all z-planes of a given cell, and this average percentage of colocalization per cell was averaged between different cells and compared between conditions. Quantitatively similar values of the percentage of colocalization were obtained if the analysis was performed in 3D (using our previously described method)⁶⁶ rather than in 2D then averaged across the cell, or conversely, if the percentage of colocalization per z-plane was summed rather than averaged, indicating that data are not biassed due to some z-plane having less GFP-positive spots than others (data not shown).

For automated quantification of the colocalization between GFP-positive nanocages and LAMP1 staining (FIG. 24a, b), we used a similar approach as the one described above to quantify the array/LAMP1 colocalization, except that the planes corresponding to the ventral side of the cell were excluded, as we noticed that nanocages had a tendency to stick to the dish, and thus when seeing a nanocage on the ventral plane of the cell, we could not know if it was bound to the cell surface, but not internalized, or simply stuck onto the dish. In addition, in this case, we expressed the percentage of colocalization as the fraction of signals that do colocalize, that is:

$$\% of colocalization = \frac{\sum Intensity colocalizing particles}{\sum Intensity total particles} \times 100$$

Indeed, as 60-mer are internalized, they accumulate in lysosomes, which thus display more signal than isolated 60-mer. Using a particle based calculation would thus not be accurate.

For automated quantification of the fraction of GFP-positive arrays associated with WGA-positive plasma membranes (FIG. 5.n, o), the raw data consisted of 3D confocal stacks (Δz=200 nm) of cells in both channels (GFP/WGA). To automatically segment the membrane channel, we used an unbiased intensity threshold set at the mean plus one standard deviation of the WGA signal intensity distribution in the brightest plane after homogeneous background subtraction. We then measured the intensity of the GFP channel either for each z-plane in the entire cell, or within the membrane segmented regions. To avoid noise, we measured GFP intensities only above an intensity threshold set automatically to the mean plus two standard deviations of the GFP signal intensity distribution in the brightest plane (after homogenous background subtraction). We then scored for each z-plane the percentage of internalized signal as the fraction of the total signal not associated with membrane, that is:

$$\% of internalized signal = \frac{Integrated {intensity}_{whole cell} - Integrated {intensity}_{membrane}}{Integrated {intensity}_{whole cell}} \times 100$$

This measurement was then averaged for all z-planes of a given cell, and this average percentage of colocalization per cell was averaged between different cells and compared between conditions.

### REFERENCES

1. Sleytr, U. B., Schuster, B., Egelseer, E.-M. & Pum, D. S-layers: principles and applications. FEMS Microbiol. Rev. 38, 823-864 (2014).
2. Zhu, C, et at. Diversity in S-layers. Prog. Biophys. Mol. Biol. 123, 1-15 (2017).
3. Gonen, S., DiMaio, F., Gonen, T. & Baker, D. Design of ordered two-dimensional arrays mediated by noncovalent protein-protein interfaces. Science 348, 1365-1368 (2015).
4. Liljeström, V., Mikkilä, J. & Kostiainen, M. A. Self-assembly and modular functionalization of three-dimensional crystals from oppositely charged proteins. Nat. Commun. 5, (2014).
5. Alberstein, R., Suzuki, Y., Paesani, F. & Tczcan, F. A. Engineering the Entropy-Driven Free-Energy Landscape of a Dynamic, Nanoporous Protein Assembly. Nat. Chem. 10, 732-739 (2018).
6. Engineering the S-Layer of Caulobacter crescentus as a Foundation for Stable, High-Density, 2D Living Materials—Google Search. Web site: .google.com/search?q=Engineering+the+S-Layer+of+Caulobacter+crescentus+as+a+Foundation+for+Stable %2C+High-Density %2C+2D+Living+Materials&rlz=1C5CHFA_enUS807US808&oq=Engineering+the+S-Layer*of+Caulobacter+crescentus+as+a+Foundation+for+Stable %2C+High-Density %2C+2D+Living+Materials&aqs=chrome.69i57.334j0j7&sourceid=chrome&ie=UTF-8.
7. Comerci, C. J. et al. Topologically-guided continuous protein crystallization controls bacterial surface layer self-assembly. Nat. Commun. 10, 1-10 (2019).
8. Sinclair, J. C., Davies, K. M., Vénien-Bryan, C. & Noble, M. E. M. Generation of protein lattices by fusing proteins with matching rotational symmetry. Nat. Nanotechnol. 6, 558-562(2011).
9. Vantomme, G. & Meijer, E. W. The construction of supramolecular systems. Science 363, 1396-1397 (2019).
10. Bale, J. B. et al. Accurate design of megadalton-scale two-component icosahedral protein complexes. Science 353, 389-394 (2016).
11. Butterfield, G. L. et al. Evolution of a designed protein assembly encapsulating its own RNA genome. Nature 552, 415-420 (2017).
12. Marcandalli, J. et al. Induction of Potent Neutralizing Antibody Responses by a Designed Protein Nanoparticle Vaccine for Respiratory Syncytial Virus. Cell 176, 1420-1431.e17 (2019).
13. Tan, R., Zhu, H., Cao, C. & Chen, O. Multi-component superstructures self-assembled from nanocrystal building blocks. Nanoscale 8, 9944-9961 (2016).
14. Dryden, K. A., Crowley, C. S., Tanaka, S., Yeates, T. O. & Yeager, M. Two-dimensional crystals of carboxysome shell proteins recapitulate the hexagonal packing of three-dimensional crystals. Protein Sci. Publ. Protein Soc. 18, 2629-2635 (2009).
15. von Kügelgen, A. et al. In Situ Structure of an Intact Lipopolysaccharide-Bound Bacterial Surface Layer. Cell 180, 348-358.e15 (2020).
16. Herrmann, J. et al. A bacterial surface layer protein exploits multistep crystallization for rapid self-assembly. Proc. Natl Acad. Sci. 117, 388-394 (2020).
17. Yeates, T. O. Geometric Principles for Designing Highly Symmetric Self-Assembling Protein Nanomaterials. Annu. Rev. Biophys. 46, 23-42 (2017).
18. Yeates, T. O., Liu, Y. & Laniado, J. The design of symmetric protein nanomaterials comes of age in theory and practice. Curr. Opin. Struct. Biol. 39, 134-143 (2016).
19. Mathaei, J. F. et al. Designing Two-Dimensional Protein Arrays through Fusion of Multimers and Interface Mutations. Nano Lett. 15, 5235-5239 (2015).
20. Garcia-Scisdedos, H., Empereur-Mot, C., Elad, N. & Levy, E. D. Proteins evolve on the edge of supramolecular self-assembly. Nature 548, 244-247 (2017).
21. Suzuki, Y. et al. Self-assembly of coherently dynamic, auxetic, two-dimensional protein crystals. Nature 533, 369-373 (2016).
22. Du, M. et al. Precise Fabrication of De Novo Nanoparticle Lattices on Dynamic 2D Protein Crystalline Lattices. Nano Lett. (2019) doi:10.1021/acs.nanolett.9b04574.
23. King, N. P. et al. Accurate design of co-assembling multi-component protein nanomaterials. Nature 510, 103-108 (2014).
24. Berman, H. M. et al. The Protein Data Bank. Nucleic Acids Res. 28, 235-242 (2000).
25. DiMaio, F., Leaver-Fay, A., Bradley, P., Baker, D. & André, 1. Modeling Symmetric Macromolecular Structures in Rosetta3. PLoS ONE 6, (2011).
26. Fleishman, S. J. et al. RosettaScripts: A Scripting Language Interface to the Rosetta Macromolecular Modeling Suite. PLOS ONE 6, e20161 (2011).
27. Goldenzweig, A. et al. Automated Structure- and Sequence-Based Design of Proteins for High Bacterial Expression and Stability. Mol. Cell 63, 337-346 (2016).
28. Chandrasckhar, S. Stochastic Problems in Physics and Astronomy. Rev. Mod. Phys. 15, 1-89 (1943).
29. Zakeri, B. et al. Peptide tag forming a rapid covalent bond to a protein, through engineering a bacterial adhesin. Proc. Natl. Acad. Sci. 109, E690-E697 (2012).
30. Hsia, Y. et al. Design of a hyperstable 60-subunit protein icosahedron. Nature 535, 136-139 (2016).
31. Pedersen, M. W. et al. Sym004: A Novel Synergistic Anti-Epidermal Growth Factor Receptor Antibody Mixture with Superior Anticancer Efficacy. Cancer Res. 70, 588-597 (2010).
32. Heukers, R. et al. Endocytosis of EGFR requires its kinase activity and N-terminal transmembrane dimerization motif. J. Cell Sci. 126, 4900-4912 (2013).
33. Chew, H. Y. et al. Endocytosis Inhibition in Humans to Improve Responses to ADCC-Mediating Antibodies. Cell 180, 895-914.e27 (2020).
34. González, L. M., Mukhitov, N. & Voigt, C. A. Resilient living materials built by printing bacterial spores. Nat. Chem. Biol. 16, 126-133 (2020).
35. Chaudhury, S., Lyskov, S. & Gray, J. J. PyRosetta: a script-based interface for implementing molecular modeling algorithms using Rosetta. Bioinformatics 26, 689-69 (2010).
36. Huang, P.-S. et al. RosettaRemodel: A Generalized Framework for Flexible Backbone Protein Design. PLOS ONE 6, e24109 (2011).
37. Hoover, D. M. & Lubkowski, J. DNAWorks: an automated method for designing oligonucleotides for PCR-based genc synthesis. Nucleic Acids Res. 30, e43 (2002).
38. Gaspar, P., Moura, G., Santos, M. A. S. & Oliveira. J. L. mRNA secondary structure optimization using a correlated stem-loop prediction. Nucleic Acids Rev. 41, e73-e73 (2013).
39. Zadeh, J. N. et al. NUPACK: Analysis and design of nucleic acid systems. J. Comput. Chem. 32, 170-173 (2011).
40. Gibson, D. G. et al. Enzymatic assembly of DNA molecules up to several hundred kilobases. Nat. Methods 6, 343-345 (2009).
41. Demonte, D., Dundas, C. M. & Park, S. Expression and purification of soluble monomeric streptavidin in Escherichia coli. Appl. Microbiol. Biotechnol. 98, 6285-6295 (2014).
42. Boer, E. de et at. Efficient biotinylation and single-step purification of tagged transcription factors in mammalian cells and transgenic mice. Proc. Natl. Acad. Sci. 100, 7480-7485 (2003).
43. Kirchofer, A. et al. Modulation of protein properties in living cells using nanobodies. Nat. Struct. Mol. Biol. 17, 133-138 (2010).
44. Sevier, C. S., Weisz, O. A., Davis, M. & Machamer, C. E. Efficient export of the vesicular stomatitis virus G protein from the endoplasmic reticulum requires a signal in the cytoplasmic tail that includes both tyrosine-based and di-acidic motifs. Mol. Biol. Cell 11, 13-22 (2000).
45. Nishimura, N. & Bach, W. E. A di-acidic signal required for selective export from the endoplasmic reticulum. Science 277, 556-558 (1997).
46. Bindels, D. S. et al. mScarlet: a bright monomeric red fluorescent protein for cellular imaging. Nat. Methods 14, 53-56 (2017).
47. Derivery, E. et al. The Arp2/3 activator WASH controls the fission of endosomes through a large multiprotein complex. Dev Cell 17, 712-723 (2009).
48. Sladitschek, H. L. & Neveu, P. A. MXS-Chaining: A Highly Efficient Cloning Platform for Imaging and Flow Cytometry Approaches in Mammalian Systems. PLOS ONE 10, e0124958 (2015).
49. Boersma, Y. L., Chao, G., Steiner, D., Wittrup, K. D. & Plückthun, A. Bispecific designed ankyrin repeat proteins (DARPins) targeting epidermal growth factor receptor inhibit A431 cell proliferation and receptor recycling. J. Biol. Chem. 286, 41273-41285 (2011).
50. Suloway, C. et al. Automated molecular microscopy: the new Leginon system. J. Struct. Biol. 151, 41-60 (2005).
51. Scheres, S. H. W. RELION: Implementation of a Bayesian approach to cryo-EM structure determination. J. Struct. Biol. 180, 519-530 (2012).
52. Hura, G. L. et al. Robust, high-throughput solution structural analyses by small angle X-ray scattering (SAXS). Nat. Methods 6, 606-612 (2009).
53. Schneidman-Duhovny, D., Hammel, M., Tainer, J. A. & Sali, A. FoXS, FoXSDock and MultiFoXS: Single-state and multi-state structural modeling of proteins and their complexes based on SAXS profiles. Nucleic Acids Res. 44, W424-W429 (2016).
54. Drenth, J. Principles of Protein X-Ray Crystallography. (Springer-Verlag, 2007). doi:10.1007/0-387-33746-6.
55. CCP4 Program Suite. http://www.ccp4.ac.uk/html/unique.html.
56. Feigin, L. A. & Svergun, D. I. Structure Analysis by Small-Angle X-Ray and Neutron Scattering. (Springer US, 1987). doi:10.1007/978-1-4757-6624-0.
57. Malecki, M. J. et al. Leukemia-Associated Mutations within the NOTCH1 Heterodimerization Domain Fall into at Least Two Distinct Mechanistic Classes. Mol. Cell. Biol. 26, 4642-4651 (2006).
58. Chiaruttini, N. et al. Relaxation of Loaded ESCRT-III Spiral Springs Drives Membrane Deformation. Cell 163, 866-879 (2015).
59. Young, L. J., Ströhl, F. & Kaminski. C. F. A Guide to Structured Illumination TIRF Microscopy at High Speed with Multiple Colors. J. Vis. Exp. JoVE (2016) doi:10.3791/53988.
60. Ovesny, M., Krízek, P., Borkovec, J., Svindrych, Z. & Hagen, G. M. ThunderSTORM: a comprehensive ImageJ plug-in for PALM and STORM data analysis and super-resolution imaging. Bioinforma. Oxf. Engl. 30, 2389-2390 (2014).
61. Picco, A., Mund, M., Ries, J., Nédélec, F. & Kaksonen, M. Visualizing the functional architecture of the endocytic machinery. eLfie 4, e04535 (2015).
62. Schindelin, J. et al. Fiji: an open-source platform for biological-image analysis. Nat. Methods 9, 676-682(2012).
63. Allan, C. et al. OMERO: flexible, model-driven data management for experimental biology. Nar. Methods 9, 245-253 (2012).
64. Machado, S., Mercier, V. & Chiaruttini, N. LimeSeg: a coarse-grained lipid membrane simulation for 3D image segmentation. BMC Bioinformatics 20, (2019).
65. Bolic, S. & Cordeliéres, F. P. A guided tour into subcellular colocalization analysis in light microscopy. J. Microsc. 224, 213-232 (2006).
66. Polarized endosome dynamics by spindle asymmetry during asymmetric cell division|Nature. Web site: nature.com/articles/nature16443.

1. A two-dimensional protein structure, comprising a first polypeptide and a second polypeptide, wherein
(I)(a) the first polypeptide and the second polypeptide are different;
(b) the first polypeptide self-assembles into a first homo-oligomer, wherein the first homo-oligomer comprises a first interface region, said first interface region having a rotational symmetry;
(c) the second polypeptide self-assembles into a second homo-oligomer, wherein the second homo-oligomer comprises a second interface region, said second interface region having a rotational symmetry; and
(d) the first homo-oligomer and the second homo-oligomer interact via the first interface region and the second interface region to form a rigid interface; or
(II)(a) the first polypeptide and the second polypeptide are different;
(b) the first polypeptide self-assembles into a first homo-oligomer;
(c) the second polypeptide self-assembles into a second homo-oligomer;
(d) the first homo-oligomer and the second homo-oligomer interact to form a rigid interface; and wherein
(e) one or both of the first homo-oligomer and the second homo-oligomer has a cyclic pseudo-dihedral symmetry.

2.-5. (canceled)

6. The polypeptide of claim 1, wherein the interface comprises (a) a region of the first polypeptide within 25 amino acids from the first polypeptide C-terminus, and (b) a region of the second polypeptide within 25 amino acids from the second polypeptide N-terminus.

7. The 2D protein material of claim 1, wherein
(a) the first polypeptide comprises a secondary structure as shown below, wherein positions in parentheses are optional and may be present or absent:
First polypeptide
(LLLLLLLLLLLLLL)LLLLLLHHHLLLHHHHLLLLLLLLLHHHHHHHHH
HHHHHHHLLLHHHHHHHHHHHLLHHHHHHHHHHHHLLLLLLLLLHHHHHH
HHHLHHHHHHHHHHHHHHHHLLLLHHHHHLLLLLLLLLLLLLLLLLLLLL
HHHHHHHHHHHHHHHHLHHHHHHHHHHHHHHHHHHHHHLLLLHHHHHHHH
HHHHHHHHHHHHLHHHHHHHHHHHHHHHHHLL;
and
(b) the second polypeptide comprises a secondary structure as shown below
Second polypeptide
LLHHHHHHHHHHHHHHHHHHHHHHHHHHHHHHHHHHHHHHLLLLEEEEEL
HHHHHHHHHHHHHHHLLLLLLLLLLLEEELLLLHHHHHHHHHHLLHHHLL
HHHHHHHLLLLLEEEEEELLLLLHHHHHHHHHHHHLLLEEEEEEELLLHH
HHHHLLEEEEELLLLHHHHHHHHHHHHHHHHHHHHHHL
wherein H represents amino acid residues present in an alpha helix; L represents amino acids present in a loop, and E represents amino acid residues present in a beta sheet, and wherein amino acid insertions may be present in loop regions.

8.-11. (canceled)

12. A polypeptide comprising an amino acid sequence having at least 50% sequence identity to the amino acid sequence of SEQ ID NO:1, wherein the polypeptide includes a mutation at 1, 2, 3, 4, 5, 6, 7, 8, 9, 10, 11, 12, 13, 14 or all 15 positions selected from the group consisting of T210, A213, Q215, Q216, Q217, Q219, K220, K222, A223, E224, F225, A226, Q227, Q229, and K230 relative to SEQ ID NO:1, wherein residues in parentheses are optional and may be present or absent.
1d2t
(SEQ ID NO: 1)
(LALVATGNDTTTKP)DLYYLKNSEAINSLALLPPPPAVGSIAFLNDQAM
YEQGRLLRNTERGKLAAEDANLSSGGVANAFSGAFGSPITEKDAPALHKL
LTNMIEDAGDLATRSAKDHYMRIRPFAFYGVSTCNTTEQDKLSKNGSYPS
GHTSIGWATALVLAEINPQRQNEILKRGYELGQSRVICGYHWQSDVDAAR
VVGSAVVATLHTNPAFQQQLQKAKAEFAQHQK

13. (canceled)

14. The polypeptide of claim 12, wherein mutations in the polypeptide relative to SEQ ID NO:1 comprise:
(a) 1, 2, 3, 4, or all 5 of A213E, Q216A, Q219I, A223I, A226K;
(b) 1, 2, 3, 4, 5, 6, 7, 8, 9, 10, 11, or all 12 of Q215I, Q216A, Q217A, Q219I, K222L, A223I, E224L, F225T, A226H, Q227R, Q229R, K230T;
(c) 1, 2, 3, 4, 5, 6, 7, 8, 9, 10, 11, 12, or all 13 of T210R, Q215I, Q216A, Q217A, Q219I, K222L, A223L, E224L, F225T, A226Y, Q227R, Q229R, K230T;
(d) 1, 2, 3, 4, 5, 6, 7, 8, 9, 10, 11, 12, or all 13 of A213K, Q215I, Q216S, Q217A, Q219I, K222L, A223L, E224L, F225T, A226V, Q227R, Q229R, K230T;
(e) 1, 2, 3, 4, 5, 6, 7, 8, 9, 10, 11, 12, 13, or all 14 of T210R, A213K, Q215I, Q216S, Q217A, Q219I, K222L, A223L, E224L, F225T, A226Y, Q227R, Q229R, K230T;
(f) 1, 2, 3, 4, 5, 6, 7, 8, 9, 10, 11, 12, or all 13 of A213K, Q215I, Q216S, Q217A, Q219I, K222L, A223L, E224L, F225T, A226Y, Q227R, Q229R, K230T;
(g) 1, 2, 3, 4, 5, 6, 7, 8, 9, 10, 11, 12, or all 13 of A213K, Q215I, Q216S, Q217A, Q219I, K222L, A223L, E224L, F225T, A226H, Q227R, Q229R, K230T;
(h) 1, 2, 3, 4, 5, 6, 7, 8, 9, 10, or all 11 of A213E, Q216A, Q219L, K222L, A223I, E224L, F225T, A226Y, Q227R, Q229R, K230T;
(i) 1, 2, 3, 4, 5, 6, 7, 8, 9, 10, or all 11 of A213E, Q216A, Q219L, K222L, A223I, E224L, F225T, A226H, Q227R, Q229R, K230T;
(j) 1, 2, 3, 4, 5, 6, 7, 8, 9, 10, 11, or all 12 of T210R, A213E, Q216A, Q219I, K222L, A223I, E224L, F225T, A226Y, Q227R, Q229R, K230T;
(k) 1, 2, 3, 4, 5, 6, 7, 8, 9, 10, 11, or all 12 of A213K, Q215I, Q216S, Q217A, Q219I, K220E, K222L, A223L, A226L, Q227E, Q229R, K230Q;
(l) 1, 2, 3, 4, 5, 6, 7, 8, 9, or all 10 of A213E, Q216A, Q219L, K220E, K222L, A223L, A226L, Q227E, Q229R, K230Q;
(m) 1, 2, 3, 4, 5, 6, or all 7 of Q215I, Q216A, Q217A, Q219I, A223I, E224L, Q227E;
(n) 1, 2, 3, 4, 5, 6, 7, or all 8 of A213K, Q215I, Q216S, Q217A, Q219I, A223L, E224L, Q227E;
(o) 1, 2, 3, 4, 5, 6, 7, or all 8 of A213K, Q215I, Q216S, Q217A, Q219I, A223L, E224L, Q227E;
(p) 1, 2, 3, 4, 5, or all 6 of A213D, Q217A, Q219I, K222L, A223L, Q227E;
(q) 1, 2, 3, 4, 5, 6, or all 7 of A213D, Q217A, Q219I, K222L, A223L, A226H, Q227E;
(r) 1, 2, 3, 4, 5, 6, 7, 8, or all 9 of A213K, Q215R, Q216S, Q217N, Q219L, K220R; A223I, A226K, Q227R
(s) 1, 2, 3, 4, 5, 6, 7, 8, or all 9 of A213K, Q215R, Q216S, Q217N, Q219I, K220R, A223I, A226T, Q227R;
(t) 1, 2, 3, 4, 5, 6, 7, or all 8 of A213K, Q215R, Q216S, Q217N, Q219L, K220R, A223I, Q227R;
(u) 1, 2, 3, 4, 5, 6, 7, 8, or all 9 of A213K, Q215R, Q216S, Q217N, Q219L, K220R, A223I, A226K, Q227R;
(v) 1, 2, 3, 4, 5, 6, 7, 8, or all 9 of A213E, Q215L, Q216S, Q217N, Q219L, K220E, A223I, A226V, Q227D; or
(w) 1, 2, 3, 4, 5, 6, 7, or all 8 of A213E, Q216V, Q219I, K220E, K222L, A223E, A226T, Q227E.

15. The polypeptide of claim 12, wherein mutations in the polypeptide comprise mutations at 1, 2, 3, 4, 5, 6, 7, 8, 9, 10, 11, 12, 13, 14, 15, 16, or all 17 residues selected from residues 10, 65, 72, 73, 74, 77, 81, 85, 89, 90, 96, 100, 119, 152, 157, 167, and 197 relative to SEQ ID NO:1.

16. The polypeptide of claim 15, wherein mutations in the polypeptide comprise 1, 2, 3, 4, 5, 6, 7, 8, 9, 10, 11, 12, 13, 14, 15, 16, or all 17 mutations selected from the group consisting of 10A, 65Q, 72P, 73E, 74Q or 74H, 77K, 81C, 85F, 89P, 90E, 96Y, 100R, 119Q, 152A, 157M or 157F, 167D, and 197G relative to SEQ ID NO:1.

17. The polypeptide of claim 12, wherein the polypeptide comprises an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity to the amino acid sequence selected from the group consisting of SEQ ID NOS:2-3I, wherein residues in parentheses may be present or absent.

18. (canceled)

19. The polypeptide of claim 17, further comprising one or more additional functional peptide domains.

20. The polypeptide of claim 19, wherein the polypeptide comprises an amino acid sequence at least 50% sequence identity to the amino acid sequence of a sequence selected from the group consisting of SEQ ID NO:32-47, wherein residues in parentheses are optional and may be present or absent.

21. (canceled)

22. A homo-oligomer of the polypeptide of claim 12.

23.-25. (canceled)

26. A polypeptide comprising an amino acid sequence having at least 50% sequence identity to the amino acid sequence of SEQ ID NO: 100, wherein the polypeptide includes a mutation at 1, 2, 3, 4, 5, 6, 7, 8, 9, 10, 11, 12, 13, or all 14 positions selected from the group consisting of M1, N5, E8, K9, Q12, E13, H14, K16, I17, V18, Q19, A20, E22, and I23 relative to SEQ ID NO:100.
1tk9
(SEQ ID NO: 100)
MSLINLVEKEWQEHQKIVQASEILKGQIAKVGELLCECLKKGGKILICGN
GGSAADAQHFAAELSGRYKKERKALAGIALTTDTSALSAIGNDYGFEFVF
SRQVEALGNEKDVLIGISTSGKSPNVLEALKKAKELNMLCLGLSGKGGGM
MNKLCDHNLVVPSDDTARIQEMHILIIHTLCQIIDESF

27. The polypeptide of claim 26 wherein mutations in the polypeptide relative to SEQ ID NO:100 comprise:
(a) 1, 2, 3, 4, 5, or all 6 of N5T, K9L, Q12L, K16L, A20L, I23R
(b) 1, 2, 3, 4, 5, 6, 7, 8, 9, 10, 11, 12, or all 13 of M1S, N5A, E8H, K9L, Q12L, H14A, K16L, I17A, V18T, Q19V, A20L, E22S, I23S
(c) 1, 2, 3, 4, 5, 6, 7, 8, 9, 10, 11, 12, or all 13 of M1S, N5A, E8Y, K9L, Q12L, H14A, K16L, I17A, V18T, Q19V, A20L, E22S, I23S
(d) 1, 2, 3, 4, 5, 6, 7, 8, 9, 10, 11, or all 12 of M1S, N5A, K9L, Q12L, H14A, K16L, I17A, V18T, Q19V, A20N, E22S, I23D
(e) 1, 2, 3, 4, 5, 6, 7, 8, 9, 10, 11, 12, or all 13 of M1S, N5A, E8Y, K9L, Q12L, H14A, K16L, I17A, V18T, Q19V, A20N, E22S, I23D
(g) 1, 2, 3, 4, 5, 6, 7, 8, 9, 10, 11, 12, or all 13 of M1S, N5A, E8H, K9L, Q12L, H14A, K16L, I17A, V18T, Q19V, A20N, E22S, I23D
(h) 1, 2, 3, 4, 5, 6, 7, 8, 9, 10, 11, or all 12 of M1S, N5A, E8Y, K9L, Q12L, H14A, K16L, I17A, V18T, A20L, E22S, I23R
(i) 1, 2, 3, 4, 5, 6, 7, 8, 9, 10, 11, or all 12 of M1S, N5A, E8H, K9L, Q12L, H14A, K16L, I17A, V18T, A20L, E22S, I23R
(k) 1, 2, 3, 4, 5, 6, 7, 8, 9, or all 10 of M1S, N5T, K9R, Q12L, E13R, K16L, I17A, A20N, E22S, I23D
(l) 1, 2, 3, 4, 5, 6, 7, 8, 9, or all 10 of M1S, N5T, K9R, Q12L, E13R, K16L, I17A, A20L, E22S, I23R
(m) 1, 2, 3, 4, 5, 6, 7, 8, 9, 10, or all 11 of M1S, N5A, K9L, Q12L, E13R, K16L, I17A, Q19V, A20L, E22S, I23S
(n) 1, 2, 3, 4, 5, 6, 7, 8, 9, 10, or all 11 of M1S, N5A, K9L, Q12L, E13R, K16L, I17A, Q19V, A20D, E22S, I23D
(o) 1, 2, 3, 4, 5, 6, 7, 8, 9, 10, or all 11 of M1S, N5A, K9L, Q12L, E13R, K16L, I17A, Q19V, A20N, E22S, I23D
(p) 1, 2, 3, 4, 5, 6, 7, or all 8 of M1A, N5Q, K9L, Q12I, E13K, K16L, E22A, I23R
(q) 1, 2, 3, 4, 5, 6, 7, 8, or all 9 of M1A, N5Q, E8H, K9L, Q12I, E13K, K16L, E22A, I23R
(r) 1, 2, 3, 4, 5, 6, 7, 8, 9, 10, or all 11 of M1S, N5T, K9L, Q12L, E13A, K16L, I17A, Q19E, A20L, E22S, I23S
(s) 1, 2, 3, 4, 5, 6, 7, 8, 9, or all 10 of M1S, N5T, K9E, Q12L, K16L, I17A, Q19E, A20L, E22S, I23S
(t) 1, 2, 3, 4, 5, 6, 7, 8, 9, 10, or all 11 of M1S, N5T, K9E, Q12L, E13A, K16L, I17A, Q19E, A20D, E22S, I23S
(u) 1, 2, 3, 4, 5, 6, 7, 8, 9, 10, or all 11 of M1S, N5T, K9E, Q12L, E13A, K16L, I17A, Q19E, A20N, E22S, I23S
(v) 1, 2, 3, 4, 5, 6, 7, 8, or all 9 of M1K, N5Q, Q12L, E13K, K16L, I17A, Q19V, A20R, I23R
(w) 1, 2, 3, 4, 5, 6, 7, 8, 9, or all 10 of M1A, N5Q, K9L, Q12I, E13K, K16L, I17A, A20R, E22A, I23R

28. The polypeptide of claim 26, wherein mutations in the polypeptide comprise mutations at 1, 2, 3, 4, 5, 6, 7, 8, 9, 10, 11, 12, 13 or all 14 residues selected from residues 37, 38, 41, 98, 101, 111, 134, 137, 141, 150, 153, 158, 187, 189, and 190 relative to SEQ ID NO:100.

29. The polypeptide of claim 28, wherein mutations in the polypeptide comprise 1, 2, 3, 4, 5, 6, 7, 8, 9, 10, 11, 12, 13, or all 14 residues selected from residues 37R, 38A, 41N, 98Y, 101A, 111G, 134R, 137G, 141I, 150K, 153D, 158C, 187A, 189E, and 190L relative to SEQ ID NO:100.

30.-35. (canceled)

36. A homo-oligomer of the polypeptide of claim 26.

37.-39. (canceled)

40. A nucleic acid encoding the polypeptide of claim 1.

41. An expression vector comprising the nucleic acid of claim 40 operatively linked to a suitable promoter or other control sequence.

42. A host cell comprising the expression vector of claim 41.

43. (canceled)

44. A 2D protein material comprising a first homo-oligomer comprising the homo-oligomer of claim 22 and a second homo-oligomer that interact at a rigid interface, wherein the first and second homo-oligomers comprise a pair of homo-oligomers comprising an amino acid sequence having at least 50% sequence identity to the amino acid sequence selected from the group consisting of the following, wherein optional residues (including any N-terminal methionine residues) may be present or absent:
(a) SEQ ID NOS:2-7 (As1-As3), and SEQ ID NOS:50-59 (B-B4);
(b) Di_13_0A (SEQ ID NO:8) and Di_13_0B (SEQ ID NO:60);
(c) Di_13_1A (SEQ ID NO:9) and Di_13_1B (SEQ ID NO:61);
(d) Di_13_2A (SEQ ID NO:10) and Di_13_2B (SEQ ID NO:62);
(e) Di_13_3A (SEQ ID NO:11) and Di_13_3B (SEQ ID NO:63);
(f) Di_13_4A (SEQ ID NO:12) and Di_13_4B (SEQ ID NO:64);
(g) Di_13_5A (SEQ ID NO:13) and Di_13_5B (SEQ ID NO:65);
(h) Di_13_6A (SEQ ID NO:14) and Di_13_6B (SEQ ID NO:66);
(i) Di_13_7A (SEQ ID NO:15) and Di_13_7B 9SEQ ID NO:67);
(j) Di_13_8A (SEQ ID NO:16) and Di_13_8B (SEQ ID NO:68);
(k) Di_13_9A (SEQ ID NO:17) and Di_13_9B (SEQ ID NO:69);
(l) Di_13_10A (SEQ ID NO:18) and Di_13_10B (SEQ ID NO:70);
(m) Di_13_11A (SEQ ID NO:19) and Di_13_11B (SEQ ID NO:71);
(n) Di_13_12A (SEQ ID NO:20) and Di_13_12B (SEQ ID NO:72);
(o) Di_13_13A (SEQ ID NO:21) and Di_13_13B (SEQ ID NO:73);
(p) Di_13_14A (SEQ ID NO:22) and Di_13_14B (SEQ ID NO:74);
(q) Di_13_15A (SEQ ID NO:23) and Di_13_15B (SEQ ID NO:75);
(r) Di_13_16A (SEQ ID NO:24) and Di_13_16B (SEQ ID NO:76);
(s) Di_13_17A (SEQ ID NO:25) and Di_13_17B (SEQ ID NO:77);
(t) Di_13_18A (SEQ ID NO:26) and Di_13_18B (SEQ ID NO:78);
(u) Di_13_19A (SEQ ID NO:27) and Di_13_19B (SEQ ID NO:79);
(v) Di_13_20A (SEQ ID NO:28) and Di_13_20B (SEQ ID NO:80);
(w) Di_13_21A (SEQ ID NO:29) and Di_13_21B (SEQ ID NO:81);
(x) Di_13_22A (SEQ ID NO:30) and Di_13_22B (SEQ ID NO:82); and
(y) Cyclic A comp. (SEQ ID NO:31) and Cyclic B comp. (SEQ ID NO:101).

45.-46. (canceled)
